(19) United States
(12) Patent Application Publication
(10) Pub. No.: US 2026/0241170 A1
(43) Pub. Date: Aug. 20, 2026

(54) SYSTEMS AND METHODS FOR MULTIMODAL ELECTRICAL STIMULATION

(71) Applicant: SPR THERAPEUTICS, INC., Cleveland, OH (US)

(72) Inventors: Joseph W. BOGGS, Chapel Hill, NC (US); William H. CLARK, Cleveland, OH (US); Meredith J. MCGEE, Cary, NC (US); Nathan D. CROSBY, Cleveland, OH (US); William J. HUFFMAN, Cleveland, OH (US); Robert B. STROTHER, Willoughby Hills, OH (US); Matthew G. DEBOCK, Morrisville, NC (US)

(21) Appl. No.: 19/162,433

(22) PCT Filed: Mar. 7, 2024
(86) PCT No.: PCT/US2024/018866

§ 371 Date: Sep. 5, 2025

Related U.S. Application Data
(60) Provisional application No. 63/450,468, filed on Mar. 7, 2023.

(51) Int. Cl. A61N 1/36

(52) U.S. Cl. CPC A61N 1/36071; A61N 1/36057; A61N 1/36171; A61N 1/36175; A61N 1/36178

(57) ABSTRACT

Provided are systems and methods for applying bimodal or multimodal electrical stimulation. In an embodiment, the systems and methods may include two or more stimulating electrodes or leads at two or more stimulation modes or frequencies. In an embodiment, the systems and methods may include mixing stimulation modes or frequencies of concurrent electrical stimulation at two or more electrodes or leads. For example, the systems and methods may include two or more electrodes and may include varying modes or frequencies of stimulation modes at two or more electrodes. In an embodiment, a lower stimulus frequency may be a subharmonic frequency of a highest stimulus frequency, wherein the subharmonic frequency is the highest stimulus frequency divided by an integer. The systems and methods may be used to provide electrical stimulation of a nerve or an innervated muscle and may be used to treat pain or provide other therapeutic benefits that are superior to traditional methods using a single mode of stimulation alone. The systems and methods may be used in percutaneous and fully implanted applications.

### CROSS REFERENCE TO RELATED APPLICATIONS

This application claims priority to U.S. Patent Application No. 63/450,468, filed on Mar. 7, 2023, entitled “SYSTEMS AND METHODS FOR ELECTRICAL STIMULATION AT MULTIPLE FREQUENCIES ON MULTIPLE ELECTRODES,” which is incorporated herein by reference in its entirety.

### FIELD OF INVENTION

The present invention relates generally to systems and methods for electrical stimulation of peripheral nerves with multiple modes (e.g., frequencies) on multiple electrodes or leads to generate intricate, synergistic neural signals for the purpose of providing improved pain relief.

### BACKGROUND

The electrical stimulation of nerves, often afferent nerves, to indirectly affect the stability or performance of a physiological system can provide functional and/or therapeutic outcomes, and has been used for activating target nerves to provide therapeutic relief of pain.

While existing systems and methods can provide remarkable benefits to individuals requiring therapeutic relief, many issues and the need for improvements still remain. Many techniques, such as non-narcotic analgesics described below, have been developed to treat pain, but all of them are ultimately insufficient. Psychological strategies, such as biofeedback and psychotherapy, may be used as an adjunct to other therapies but are seldom sufficient, and there are few studies demonstrating efficacy. Electrical stimulation systems have been used for the relief of pain, but widespread use of available systems is limited.

There exist both external and implantable devices for providing electrical stimulation to activate nerves and/or muscles to provide therapeutic relief of pain. These “neurostimulators” are able to provide treatment and/or therapy to individual portions of the body. The operation of these devices typically includes the use of an electrode placed either on the external surface of the skin and/or a surgically implanted electrode. In most cases, surface electrode(s), cuff-style electrode(s), paddle-style electrode(s), spinal column electrodes, and/or percutaneous lead(s) having one or more electrodes may be used to deliver electrical stimulation to the select portion of the patient's body.

The electrical stimulation of nerves may act as a therapeutic treatment to reduce pain in individuals with acute and chronic pain conditions, including but not limited to intractable neuropathic pain, post-traumatic nerve pain, post-amputation pain, postoperative pain, chronic axial back pain, leg pain, shoulder pain, joint pain, postherpetic neuralgia, and causalgia.

### SUMMARY

The following presents a summary of this disclosure to provide a basic understanding of some aspects. This summary is intended to neither identify key or critical elements nor define any limitations of embodiments or claims. This summary may provide a simplified overview of some aspects that may be described in greater detail in other portions of this disclosure. Furthermore, any of the described aspects may be isolated or combined with other described aspects without limitation.

Described are systems and methods for applying electrical stimulation of peripheral nerves. In an embodiment, the systems and methods may include stimulating through two or more electrodes and/or leads at different frequencies. As illustrated in FIG. 1, the systems and methods may include stimulating through two or more electrodes and/or leads to target one or more peripheral nerves or peripheral nerve branches and coordinate two or more signals into a composite signal that elicits central changes in the brain and to provide pain relief to systems or regions disrupted by disease, trauma, overuse, or otherwise subject to maladaptive changes. In an embodiment, the systems and methods may include mixing frequencies of concurrent electrical stimulation at multiple electrodes and/or leads. For example, the systems and methods may include more than one lead and may include varying frequencies of stimulation at one or more leads. In an embodiment, a lower stimulus frequency may be a subharmonic frequency of a highest stimulus frequency, wherein the subharmonic frequency is the highest stimulus frequency divided by an integer. The systems and methods may be used to provide electrical stimulation of a nerve or an innervated muscle and may be used to treat pain or provide other therapeutic benefits. The systems and methods may be used in percutaneous and/or fully implanted applications.

Disclosed is a multimodal peripheral nerve stimulation system. In an embodiment, the system may comprise a first lead configured for insertion into a body proximal to one or more regions of pain, wherein the first lead comprises one or more first electrodes integrally formed with the first lead and wherein the one or more first electrodes are configured to be positioned near at least one nerve or nerve branch that innervates the one or more regions of pain. In an embodiment, the system may comprise a second lead configured for insertion into a body proximal to a second one or more regions of pain, wherein the second lead comprises one or more second electrodes integrally formed with the second lead and wherein the one or more second electrodes are configured to be positioned near at least one nerve or nerve branch that innervates the second one or more regions of pain. In an embodiment, the system may comprise an electrical stimulation device operatively coupled to the first and second leads.

In an embodiment, the electrical stimulation device may be configured to apply electrical stimulation comprising a first mode of stimulation comprising a first set of stimulation parameters through the first lead during a first treatment period. In an embodiment, the electrical stimulation may comprise a second mode of stimulation comprising a second set of stimulation parameters through the second lead during a second treatment period. In an embodiment, the first and second treatment periods may overlap in time. In an embodiment, the first and second set of stimulation parameters may be different. In an embodiment, the first and second set of stimulation parameters may produce different effects on different target neural fibers to activate multiple mechanisms of pain relief that convergently modulate central nervous system plasticity associated with the one or more regions of pain and the second one or more regions of pain.

In an embodiment, the first set of stimulation parameters may comprise a first parameter of frequency. In an embodiment, the first set of stimulation parameters may comprise a second parameter of stimulation selected from a group consisting of: frequency, pulse duration, amplitude, duty cycle, pattern of stimulus pulses, polarity, a predetermined number of phases, and waveform shape. In an embodiment, the second set of stimulation parameters may comprise a third parameter selected from a group consisting of: frequency, pulse duration, amplitude, duty cycle, pattern of stimulus pulses, polarity, a predetermined number of phases, and waveform shape. In an embodiment, the first parameter of frequency may be different from the frequency of the second parameter of stimulation.

In an embodiment, the first parameter of the frequency may be provided by the one or more first electrodes and wherein the first parameter of the frequency may be a subharmonic frequency of a stimulation frequency provided by the one or more second electrodes, divided by an integer. In an embodiment, the subharmonic frequency may be continuously provided by the one or more first electrodes. In an embodiment, the subharmonic frequency may be provided at varying burst lengths by the one or more first electrodes.

In an embodiment, the one or more first electrodes may provide stimulation at a different frequency from the one or more second electrodes. In an embodiment, the one or more first electrodes may provide stimulation at an equivalent frequency from the one or more second electrodes. In an embodiment, the one or more electrodes may comprise at least three first electrodes wherein two or more of the at least three first electrodes may provide stimulation at different frequencies and one or more of the at least three first electrodes may provide stimulation at an equivalent frequency from any of the frequencies of the two or more of the at least three first electrodes. In an embodiment, the one or more electrodes may comprise at least three first electrodes wherein two or more of the at least three first electrodes may provide stimulation at different frequencies and one or more of the at least three first electrodes may provide stimulation at a different frequency from any of the frequencies of the two or more of the at least three first electrodes.

In an embodiment, modulation of stimulation frequency provided by the one or more electrodes may avoid overactivation or fatigue of one or both of the nerve and muscle. In an embodiment, a neurostimulation pulse of the electrical stimulation delivered from the one or more electrodes may be biphasic or multiphasic, and/or symmetrical or asymmetrical, and/or may be rectangular, exponential, or sinusoidal, or is a combination of rectangular, exponential, and/or sinusoidal. In an embodiment, a neurostimulation pulse of the electrical stimulation delivered from the one or more electrodes may comprise continuous or intermittent trains.

In an embodiment, a neurostimulation pulse of the electrical stimulation delivered from the one or more electrodes may comprise duty cycles that are regular and repeatable from one intermittent burst to another. In an embodiment, a neurostimulation pulse of the electrical stimulation delivered from the one or more electrodes may comprise duty cycles with each burst varying in a pre-programmed manner. In an embodiment, a neurostimulation pulse of the electrical stimulation delivered from the one or more electrodes may comprises duty cycles with each burst varying in a non-repeating manner. In an embodiment, a neurostimulation pulse of the electrical stimulation delivered from the one or more electrodes may comprise duty cycles with each burst varying in a random or pseudo random manner. In an embodiment, a neurostimulation pulse of the electrical stimulation delivered from the one or more electrodes may comprise duty cycles with each burst varying in a combination of repeatable, random or pseudo random patterns, and/or non-repeating preprogrammed patterns.

In an embodiment, a first neurostimulation pulse of the electrical stimulation delivered from the one or more electrodes may comprise avoidance of overlapping with a second neurostimulation pulse of the electrical stimulation delivered from the second one or more electrodes. In an embodiment, avoidance of overlapping of the first and second neurostimulation pulse may avoid undesirable effects of superposition while maintaining coordinated delivery of multiple modes. In an embodiment, avoidance of overlapping of the first and second neurostimulation pulse may avoid undesirable effects of muscle fatigue while maintaining coordinated delivery of multiple modes. In an embodiment, avoidance of overlapping of the first and second neurostimulation pulse may avoid undesirable effects of nerve fatigue while maintaining coordinated delivery of multiple modes. In an embodiment, avoidance of overlapping of the first and second neurostimulation pulse may avoid undesirable effects of tissue damage while maintaining coordinated delivery of multiple modes.

In an embodiment, the one or more first electrodes may deliver stimulation intended to activate large diameter primary afferent fibers. In an embodiment, the one or more second electrodes may deliver stimulation intended to activate large diameter efferent motor fibers. In an embodiment, the one or more second electrodes may deliver stimulation intended to generate indirect proprioceptive afferent signals.

Disclosed is a multimodal peripheral nerve stimulation system. In an embodiment, the system may comprise a lead configured for insertion into a body proximal to a region of pain. In an embodiment, the system may comprise two or more electrodes integrally formed with the lead, wherein the two or more electrodes may be configured to be placed near a nerve and/or nerve branch that innervates the region of pain. In an embodiment, the system may comprise an electrical stimulation device operatively coupled to the two or more electrodes and configured to apply electrical stimulation with a first set of stimulation parameters through a first of the two or more electrodes, and with a second set of stimulation parameters through a second of the two or more electrodes. In an embodiment, the first set of stimulation parameters may be different from the second set of stimulation parameters. In an embodiment, the first and second sets of stimulation parameters may produce a synergistic effect to provide pain relief to the region of pain.

In an embodiment, a frequency of the first set of stimulation parameters may be different from a frequency of the second set of stimulation parameters. In an embodiment, the frequency of the first set of stimulation parameters may be a subharmonic frequency of the frequency of the second set of stimulation parameters, divided by an integer. In an embodiment, a frequency of the first set of stimulation parameters may be equivalent to a frequency of the second set of stimulation parameters.

In an embodiment, the electrical stimulation may comprise a first and a second channel, wherein amplitude of the electrical stimulation is dynamically modulated on each of the first and second channels. In an embodiment, the electrical stimulation may comprise a first and a second channel, wherein pulse duration of the electrical stimulation is dynamically modulated on each of the first and second channels. In an embodiment, the electrical stimulation may comprises a first and a second channel, wherein frequency of the electrical stimulation is dynamically modulated on each of the first and second channels. In an embodiment, the electrical stimulation may comprise a first and a second channel, wherein duty cycle of the electrical stimulation is dynamically modulated on each of the first and second channels.

In an embodiment, neurostimulation pulses of the electrical stimulation from the two or more electrodes may be continuous or intermittent trains. In an embodiment, the lead and the electrical stimulation device may be fully implanted within the body.

Disclosed is a method. In an embodiment, the method may comprise placing a lead comprising a plurality of electrodes integrally formed with the lead into a portion of a body that is proximal to a region of pain. In an embodiment, the method may comprise positioning the plurality of electrodes near at least one nerve or nerve branch that innervates the region of pain. In an embodiment, the method may comprise electrically stimulating the nerve. In an embodiment, the electrical stimulation may comprise a first set of stimulation parameters applied through a first electrode of the plurality of electrodes. In an embodiment, the electrical stimulation may comprise a second set of stimulation parameters applied through a second electrode of the plurality of electrodes. In an embodiment, the first and second sets of stimulation parameters may produce a synergistic effect to provide pain relief to the region of pain.

In an embodiment, the electrical stimulation may recondition a centrally maintained pain state. In an embodiment, the first set of stimulation parameters may comprise a first frequency and the second set of stimulation parameters may comprise a second frequency, wherein the first and second frequencies may be are different. In an embodiment, the first set of stimulation parameters may comprise a first frequency and the second set of stimulation parameters may comprise a second frequency, wherein the first and second frequencies may be equivalent. In an embodiment, the first set of stimulation parameters may comprise a first frequency and the second set of stimulation parameters may comprise a second frequency, wherein the first and second frequencies may activate neural fibers that engage multiple mechanisms to provide pain relief.

In an embodiment, the first set of stimulation parameters may comprise a frequency of between 4-20 Hz targeting motor nerve fibers in a motor nerve or mixed motor and sensory nerve resulting in cyclical muscle twitches and/or muscle contractions and indirectly activate proprioceptive afferent pathways. In an embodiment, the second set of stimulation parameters may comprise a frequency of between 60-120 Hz to activate directly afferent pathways. In an embodiment, the electrical stimulation may engage in both indirect and direct afferent pathways via combinatory motor stimulation and sensory stimulation. In an embodiment, the electrical stimulation may engage in indirect and/or direct afferent pathways via combinatory motor stimulation and sensory stimulation. In an embodiment, the first set of stimulation parameters may comprise a stimulation frequency low enough to evoke visible muscle twitches and/or muscle contraction of a targeted muscle innervated by the nerve or nerve branch, with or without a modulating envelope.

In an embodiment, the stimulation frequency may be between 0.05 and 40 Hz. In an embodiment, the second set of stimulation parameters may comprise a frequency high enough to paresthesia in the region of pain with or without a modulating envelope. In an embodiment, the stimulation frequency may be between 12 and 500 Hz. In an embodiment, either the first or the second set of stimulation parameters may comprise a frequency high enough to produce a neural effect that is sub-perception in the region of pain with or without a modulating frequency. In an embodiment, the stimulation frequency may be between 500 and 15000 Hz. In an embodiment, either the first or the second set of stimulation parameters may be applied intermittently at frequencies to produce patient sensation and muscle twitch without muscle fatigue. In an embodiment, the frequencies may be between 12 and 1500 Hz. In an embodiment, the plurality of electrodes may provide a coordinated multimodal signal to mimic coordination between motor and sensory signals.

In an embodiment, a first electrode of the plurality of electrodes may deliver stimulation to activate large diameter primary afferent fibers. In an embodiment, a second electrode of the plurality of electrodes may deliver stimulation to activate large diameter efferent motor fibers. In an embodiment, the second electrode of the plurality of electrodes may deliver stimulation to generate indirect proprioceptive afferent signals.

In an embodiment, coordination between afferent, efferent, and proprioceptive afferent signals may avoid overlapping signals to avoid nerve fatigue. In an embodiment, coordination between afferent, efferent, and proprioceptive afferent signals may avoid overlapping signals to avoid tissue damage. In an embodiment, coordination between afferent, efferent, and proprioceptive afferent signals may avoid overlapping signals to avoid painful stimulation. In an embodiment, coordination between afferent, efferent, and proprioceptive afferent signals may avoid overlapping signals to avoid off-target activation.

Disclosed is a multimodal peripheral nerve stimulation system. The system may comprise a first lead configured for insertion into a portion of a body that is proximal to one or more regions of pain and includes one or more electrodes integrally formed and configured to be positioned inside the body near at least one nerve or nerve branch that innervates the region(s) of pain. The system may comprise a second lead also configured for insertion into a portion of a body that is proximal to one or more regions of pain and includes one or more electrodes integrally formed and configured to be positioned inside the body near at least one nerve or nerve branch that innervates the region(s) of pain. The system may comprise and an electrical stimulation device operatively coupled to the two leads and configured to apply electrical stimulation with a first mode of stimulation and set of stimulation parameters through the first lead during a first treatment period, and with a second mode of stimulation and set of stimulation parameters through the second lead during a second treatment period, where the treatment periods overlap in time and whereby the modes of stimulation parameters are different and produce different effects on different target neural fibers to activate multiple mechanisms of pain relief that convergently modulate central nervous system plasticity associated with the pain.

The system may comprise one or more (or all) of any of the following, in any combination:
wherein the electrical stimulation includes a first parameter of frequency.
wherein the electrical stimulation includes a second parameter of stimulation selected from a group consisting of: pulse duration, amplitude, duty cycle, pattern of stimulus pulses, polarity, a predetermined number of phases, and waveform shape.
wherein the electrical stimulation includes a third parameter selected from a group consisting of: frequency, pulse duration, amplitude, duty cycle, pattern of stimulus pulses, polarity, a predetermined number of phases, and waveform shape.
wherein the first parameter of frequency for one electrode is different from the first parameter of frequency for one or more other electrodes.
wherein a stimulation frequency provided by one electrode is a subharmonic frequency of a stimulation frequency provided by another electrode, divided by an integer.
wherein the subharmonic frequency is continuously provided by one or more electrodes.
wherein the subharmonic frequency is provided at varying burst lengths by one or more electrodes.
wherein the first parameter and/or second parameter and/or third parameter of one electrode is different from the first parameter and/or second parameter and/or third parameter from a one or more other electrodes.
wherein two or more leadless electrodes deliver stimulation at two or more frequencies
wherein two or more electrodes all provide stimulation at different frequencies
wherein two or more electrodes provide stimulation at the same frequencies
wherein two or more electrodes provide stimulation at different frequencies and two or more electrodes provide stimulation at the same frequencies
wherein the stimulation frequency provided by one or more electrodes is modulated to increase the effect or noticeability of the stimulation frequency provided by one or more electrodes.
wherein the modulation of stimulation frequency provided by one or more electrodes avoids overactivation or fatigue of the muscle.
wherein the modulation of stimulation frequency provided by one or more electrodes avoids overactivation or fatigue of the nerve.
wherein the modulation of stimulation frequency provided by one or more electrodes avoids overactivation or fatigue of the nerve and muscle.
wherein the stimulation provided by one or more electrodes is concurrent (e.g., stimulation pulses are delivered from the one or more electrodes to each target nerve in concurrent, overlapping, simultaneous, and/or coinciding periods of time or durations of therapy).
wherein the stimulation from two or more electrodes occurs at different times (stimulation pulses are delivered from the one or more electrode to each target nerve in non-overlapping, alternating, rotating, consecutive, or patterned sequences of periods of time or durations of therapy on the scale of milliseconds, seconds, minutes, hours, and/or days).
wherein the frequencies of stimulation are concurrent, simultaneous, alternating, in a pattern, overlapping, non-overlapping, randomly, or interleaved.
wherein the intensity of stimulation is modulated between modes
wherein the amplitude of stimulation is dynamically modulated on each individual channel.
wherein the pulse duration of stimulation is dynamically modulated on each individual channel.
wherein the frequency of stimulation is dynamically modulated on each individual channel.
wherein the duty cycle of stimulation is dynamically modulated on each individual channel.
wherein the neurostimulation pulse(s) delivered from one or more electrodes is biphasic or multiphasic, and/or symmetrical or asymmetrical, and/or is rectangular, exponential, or sinusoidal, or is a combination of rectangular, exponential, and/or sinusoidal.
wherein the neurostimulation pulse(s) from two or more electrodes are continuous or intermittent trains (frequency changes as a function of time).
wherein the neurostimulation pulses have duty cycles that are regular and repeatable from one intermittent burst to the next.
wherein the neurostimulation pulses have duty cycles with each burst varying in a pre-programmed manner.
wherein the neurostimulation pulses have duty cycles with each burst varying in a non-repeating manner.
wherein the neurostimulation pulses have duty cycles with each burst varying in a random or pseudo random manner.
wherein the neurostimulation pulses have duty cycles with each burst varying in a combination of repeatable, random (or pseudo random) patterns, and/or non-repeating preprogrammed patterns.
wherein the neurostimulation pulses from one electrode are designed to avoid overlapping the neurostimulation pulses from another electrode.
wherein the avoidance of overlapping pulses is intended to avoid the undesirable effects of superposition while maintaining the coordinated delivery of multiple modes.
wherein the avoidance of overlapping pulses is intended to avoid the undesirable effects of muscle fatigue while maintaining the coordinated delivery of multiple modes.
wherein the avoidance of overlapping pulses is intended to avoid the undesirable effects of nerve fatigue while maintaining the coordinated delivery of multiple modes.
wherein the avoidance of overlapping pulses is intended to avoid the undesirable effects of tissue damage while maintaining the coordinated delivery of multiple modes.
wherein the avoidance of overlapping pulses is intended to avoid painful stimulation while maintaining the coordinated delivery of multiple modes.
wherein the avoidance of overlapping pulses is intended to avoid off-target activation while maintaining the coordinated delivery of multiple modes.
wherein the leads are percutaneously placed and connected to an external pulse generator.
wherein the leads and pulse generator are fully implanted within the body.
wherein the first electrode delivers stimulation intended to activate large diameter primary afferent fibers.
wherein the second electrode delivers stimulation intended to activate large diameter efferent motor fibers.
wherein the second electrode delivers stimulation intended to generate indirect proprioceptive afferent signals (reafferent signals).
wherein the afferent, efferent, and reafferent signals are coordinated into a composite signal.
wherein the combinatory afferent, efferent, and reafferent signals are intended to converge in order to mimic the natural coordination between sensory and motor signals in the central nervous system, provide biomimetic signals, or provide signals outside the realm of natural sensations to produce changes in central pain processing.
wherein the afferent, efferent, and reafferent signals are intended to provide superior neural signaling to more effectively recondition the underlying pain state for superior relief.
wherein the afferent, efferent, and reafferent signals are intended to create complex, quasi-physiological (biomimetic) neural signals to more effectively reduce pain.
wherein the afferent, efferent, and reafferent signals are intended to stimulate an unhealthy nerve and/or nerve with damage or nerve with pre-existing damage.
wherein the coordination between the afferent, efferent, and reafferent signals are intended to increase the likelihood of relief.
wherein the coordination between the afferent, efferent, and reafferent signals are intended to decrease the amount of time until there is relief.
wherein the coordination between the afferent, efferent, and reafferent signals are intended to increase the proportion of individuals with relief regardless of pain etiologies.
wherein the coordination between the afferent, efferent, and reafferent signals are intended to increase the duration of relief.
wherein the coordination between the afferent, efferent, and reafferent signals are intended to provide pain relief to systems or regions disrupted by disease.
wherein the coordination between the afferent, efferent, and reafferent signals are intended to provide pain relief to systems or regions disrupted by trauma.
wherein the coordination between the afferent, efferent, and reafferent signals are intended to provide pain relief to systems or regions disrupted by overuse.
wherein the coordination between the afferent, efferent, and reafferent signals are intended to provide pain relief to systems or regions disrupted by maladaptive changes.
wherein the coordination between the afferent, efferent, and reafferent signals intends to avoid the overlapping of signals to avoid superposition of signals.
wherein the coordination between the afferent, efferent, and reafferent signals intends to avoid the overlapping of signals to avoid muscle fatigue.
wherein the coordination between the afferent, efferent, and reafferent signals intends to avoid the overlapping of signals to avoid nerve fatigue.
wherein the coordination between the afferent, efferent, and reafferent signals intends to avoid the overlapping of signals to avoid tissue damage.
wherein the coordination between the afferent, efferent, and reafferent signals intends to avoid the overlapping of signals to avoid painful stimulation.
wherein the coordination between the afferent, efferent, and reafferent signals intends to avoid the overlapping of signals to avoid off-target activation.

Disclosed is a multimodal peripheral nerve stimulation system. The system may comprise one or more leads configured for insertion into a portion of a body that is proximal to one or more regions of pain. The system may comprise two or more electrodes integrally formed with one or more leads, wherein the two or more electrodes are configured to be positioned inside the body and placed near at least one nerve or nerve branch that innervates the region(s) of pain. The system may comprise an electrical stimulation device operatively coupled to the two or more electrodes on the one or more leads to apply electrical stimulation with a set of stimulation parameters through the first electrode, and with one or more different set of stimulation parameters through the one or more additional electrodes on the same or different lead, whereby the sets of stimulation parameters are provided in various fashions to produce the synergistic effect in order to provide pain relief to systems or regions perceived as painful.

The system may comprise one or more (or all) of any of the following, in any combination:
wherein the electrical stimulation includes a first parameter of frequency.
wherein the electrical stimulation includes a second parameter of stimulation selected from a group consisting of: pulse duration, amplitude, duty cycle, pattern of stimulus pulses, polarity, a predetermined number of phases, and waveform shape.
wherein the electrical stimulation includes a third parameter selected from a group consisting of: frequency, pulse duration, amplitude, duty cycle, pattern of stimulus pulses, polarity, a predetermined number of phases, and waveform shape.
wherein the first parameter of frequency for one electrode is different from the first parameter of frequency for one or more other electrodes.
wherein a stimulation frequency provided by one electrode is a subharmonic frequency of a stimulation frequency provided by another electrode, divided by an integer.
wherein the subharmonic frequency is continuously provided by one or more electrodes.
wherein the subharmonic frequency is provided at varying burst lengths by one or more electrodes.
wherein the first parameter and/or second parameter and/or third parameter of one electrode is different from the first parameter and/or second parameter and/or third parameter from a one or more other electrodes.
wherein two or more leadless electrodes deliver stimulation at two or more frequencies
wherein two or more electrodes all provide stimulation at different frequencies
wherein two or more electrodes provide stimulation at the same frequencies
where two or more electrodes provide stimulation at different frequencies and two or more electrodes provide stimulation at the same frequencies
wherein the stimulation frequency provided by one or more electrodes is modulated to increase the effect or noticeability of the stimulation frequency provided by one or more electrodes.
wherein the modulation of stimulation frequency provided by one or more electrodes avoids overactivation or fatigue of the muscle.
wherein the modulation of stimulation frequency provided by one or more electrodes avoids overactivation or fatigue of the nerve.
wherein the modulation of stimulation frequency provided by one or more electrodes avoids overactivation or fatigue of the nerve and muscle.
wherein the stimulation provided by one or more electrodes is concurrent (e.g., stimulation pulses are delivered from the one or more electrodes to each target nerve in concurrent, overlapping, simultaneous, and/or coinciding periods of time or durations of therapy).
wherein the stimulation from two or more electrodes occurs at different times (stimulation pulses are delivered from the one or more electrode to each target nerve in non-overlapping, alternating, rotating, consecutive, or patterned sequences of periods of time or durations of therapy on the scale of milliseconds, seconds, minutes, hours, and/or days).
wherein the frequencies of stimulation are concurrent, simultaneous, alternating, in a pattern, overlapping, non-overlapping, randomly, or interleaved.
wherein the intensity of stimulation is modulated between modes
wherein the amplitude of stimulation is dynamically modulated on each individual channel.
wherein the pulse duration of stimulation is dynamically modulated on each individual channel.
wherein the frequency of stimulation is dynamically modulated on each individual channel.
wherein the duty cycle of stimulation is dynamically modulated on each individual channel.
wherein the neurostimulation pulse(s) delivered from one or more electrodes is biphasic or multiphasic, and/or symmetrical or asymmetrical, and/or is rectangular, exponential, or sinusoidal, or is a combination of rectangular, exponential, and/or sinusoidal.
wherein the neurostimulation pulse(s) from two or more electrodes are continuous or intermittent trains (frequency changes as a function of time).
wherein the neurostimulation pulses have duty cycles that are regular and repeatable from one intermittent burst to the next.
wherein the neurostimulation pulses have duty cycles with each burst varying in a pre-programmed manner.
wherein the neurostimulation pulses have duty cycles with each burst varying in a non-repeating manner.
wherein the neurostimulation pulses have duty cycles with each burst varying in a random or pseudo random manner.
wherein the neurostimulation pulses have duty cycles with each burst varying in a combination of repeatable, random (or pseudo random) patterns, and/or non-repeating preprogrammed patterns.
wherein the neurostimulation pulses from one electrode are designed to avoid overlapping the neurostimulation pulses from another electrode.
wherein the avoidance of overlapping pulses is intended to avoid the undesirable effects of superposition while maintaining the coordinated delivery of multiple modes.
wherein the avoidance of overlapping pulses is intended to avoid the undesirable effects of muscle fatigue while maintaining the coordinated delivery of multiple modes.
wherein the avoidance of overlapping pulses is intended to avoid the undesirable effects of nerve fatigue while maintaining the coordinated delivery of multiple modes.
wherein the avoidance of overlapping pulses is intended to avoid the undesirable effects of tissue damage while maintaining the coordinated delivery of multiple modes.
wherein the avoidance of overlapping pulses is intended to avoid the painful stimulation while maintaining the coordinated delivery of multiple modes.
wherein the avoidance of overlapping pulses is intended to avoid the off-target activation while maintaining the coordinated delivery of multiple modes.
wherein the leads are percutaneously placed and connected to an external pulse generator.
wherein the leads and pulse generator are fully implanted within the body.
wherein the first electrode delivers stimulation intended to activate large diameter primary afferent fibers.
wherein the second electrode delivers stimulation intended to activate large diameter efferent motor fibers.
wherein the second electrode delivers stimulation intended to generate indirect proprioceptive afferent signals (reafferent signals).
wherein the afferent, efferent, and reafferent signals are coordinated into a composite signal.
wherein the combinatory afferent, efferent, and reafferent signals are intended to converge in order to mimic the natural coordination between sensory and motor signals in the central nervous system, provide biomimetic signals, or provide signals outside the realm of natural sensations to produce changes in central pain processing.
wherein the afferent, efferent, and reafferent signals are intended to provide superior neural signaling to more effectively recondition the underlying pain state for superior relief.
wherein the afferent, efferent, and reafferent signals are intended to create complex, quasi-physiological (biomimetic) neural signals to more effectively reduce pain.
wherein the afferent, efferent, and reafferent signals are intended to stimulate an unhealthy nerve and/or nerve with damage or nerve with pre-existing damage.
wherein the coordination between the afferent, efferent, and reafferent signals are intended to increase the likelihood of relief.
wherein the coordination between the afferent, efferent, and reafferent signals are intended to decrease the amount of time until there is relief.
wherein the coordination between the afferent, efferent, and reafferent signals are intended to increase the proportion of individuals with relief regardless of pain etiologies.
wherein the coordination between the afferent, efferent, and reafferent signals are intended to increase the duration of relief.
wherein the coordination between the afferent, efferent, and reafferent signals are intended to provide pain relief to systems or regions disrupted by disease.
wherein the coordination between the afferent, efferent, and reafferent signals are intended to provide pain relief to systems or regions disrupted by trauma.
wherein the coordination between the afferent, efferent, and reafferent signals are intended to provide pain relief to systems or regions disrupted by overuse.
wherein the coordination between the afferent, efferent, and reafferent signals are intended to provide pain relief to systems or regions disrupted by maladaptive changes.
wherein the coordination between the afferent, efferent, and reafferent signals intends to avoid the overlapping of signals to avoid superposition of signals.
wherein the coordination between the afferent, efferent, and reafferent signals intends to avoid the overlapping of signals to avoid muscle fatigue.
wherein the coordination between the afferent, efferent, and reafferent signals intends to avoid the overlapping of signals to avoid nerve fatigue.
wherein the coordination between the afferent, efferent, and reafferent signals intends to avoid the overlapping of signals to avoid tissue damage.
wherein the coordination between the afferent, efferent, and reafferent signals intends to avoid the overlapping of signals to avoid painful stimulation.
wherein the coordination between the afferent, efferent, and reafferent signals intends to avoid the overlapping of signals to avoid off-target activation.

Disclosed is a multimodal peripheral nerve stimulation method. The method may comprise insertion of a first lead into a portion of a body that is proximal to one or more regions of pain and includes one or more electrodes integrally formed and configured to be positioned inside the body near at least one nerve or nerve branch that innervates the region(s) of pain, The method may comprise insertion of a second lead into a portion of a body that is proximal to one or more regions of pain and includes one or more electrodes integrally formed and configured to be positioned inside the body near at least one nerve or nerve branch that innervates the region(s) of pain. The method may comprise application of electrical stimulation with a set of stimulation parameters through the first lead, and electrical stimulation with a second set of stimulation parameters through the second lead, whereby the sets of stimulation parameters are different and produce different effects on the target neural fibers to activate multiple mechanisms of pain relief that convergently modulate central nervous system plasticity associated with the pain.

The method may comprise one or more (or all) of any of the following, in any combination:
wherein a system for applying electrical stimulation via two or more electrodes of differing frequencies (e.g., multiple modes, multimodal, bimodal) may be used in order to recondition the centrally maintained pain state.
wherein the two or more frequencies provided over two or more electrodes may be used to activate neural fibers that engage multiple mechanisms to provide pain relief, resulting in a synergistic and/or complementary effect on neural pathways and central pain processing, and more comprehensive relief (e.g., providing pain relief of one or multiple etiologies of pain in one or multiple locations of pain).
wherein one or more electrodes that provide stimulation at lower frequency(ies) (e.g., 4-20 Hz) may be used to target motor nerve fibers (e.g., motor stimulation) in a motor nerve or mixed (e.g., motor and sensory) nerve resulting in cyclical muscle twitches and/or muscle contractions and indirectly activate proprioceptive afferent pathways.
wherein a periodic, cyclical, cycled, intermittent, and/or continuous motor nerve response elicits a comfortable muscle twitch and/or contraction without unwanted fatigue.
wherein one or more electrodes may be used to provide stimulation at higher frequency(ies) (e.g., 60-120 Hz) to activate directly afferent pathways (e.g., sensory stimulation).
wherein sensory nerve responses provide a continuous paresthesia over the region of pain.
wherein two or more electrodes may be placed near the same nerve or nerve branch, engaging in both indirect and direct afferent pathways via combinatory motor stimulation and sensory stimulation.
wherein two or more electrodes may be placed near two or more nerves or nerve branches, engaging in indirect and/or direct afferent pathways via combinatory motor stimulation and sensory stimulation.
wherein two or more electrodes may be placed near one or more nerves or nerve branches, engaging in indirect and/or direct afferent pathways via combinatory motor stimulation and sensory stimulation.
wherein one or more electrodes provides a stimulation frequency low enough (e.g., 0.5-12 Hz or 0.1-20 Hz, or 0.05-40 Hz) to evoke visible muscle twitches (i.e. non-fused muscle contraction) and/or muscle contraction(s) of the targeted muscle(s) innervated by the target nerve or nerve branch, with or without a modulating envelope.
wherein one or more electrodes provides a stimulation frequency high enough (e.g. 20-120 Hz or 12-200 Hz or 200-500 Hz) to evoke sensation(s) or comfortable paresthesia(s) in the region(s) of pain or in alternate target region(s), with or without a modulating envelope.
wherein one or more electrodes provides a stimulation frequency high enough (e.g., 500-15000 Hz) to produce neural effect(s) that may be sub-perception in the region(s) of pain or in alternate target region(s), with or without a modulating frequency.
wherein a system contains more than one electrode and provides a combination of stimulation frequencies that evoke muscle twitches and/or muscle contractions in the region of pain, and/or evoke sensations or comfortable paresthesia(s) in the region of pain, and/or produce neural effects that may be sub-perception in the region of pain.
wherein a system may apply electrical stimulation wherein higher frequencies (e.g. 20-120 Hz or 12-200 Hz or 200-500 Hz or 500-1500 Hz) are applied intermittently (e.g., with a duty cycle less than 100%) at lower frequencies to produce patient sensation and muscle twitch without muscle fatigue.
wherein a system applies electrical motor stimulation at the cervical medial branch of the dorsal ramus and sensory stimulation at the occipital nerve for the treatment of pain in the head and neck.
wherein a system applies electrical motor stimulation at the lumbar medial branch of the dorsal ramus and sensory stimulation at the spinal nerve for the treatment of pain in the back and legs.
wherein a system applies electrical motor stimulation at the lumbar medial branch of the dorsal ramus and sub-perception stimulation at the lumbar medial branch of the dorsal ramus for the treatment of pain in the back and legs.
wherein a system applies motor stimulation at the axillary nerve at the deltoid and sensory stimulation at the suprascapular nerve for the treatment of pain in the shoulder.
wherein a system motor stimulation at the femoral nerve more distally (e.g., at the vastus medialis oblique) and sensory stimulation at the femoral nerve more proximally (e.g., just distal to the inguinal crease) for the treatment of pain in the knee.
wherein a system with two or more electrodes provides a coordinated multimodal (e.g., bimodal) signal to mimic the natural coordination between motor and sensory signals resulting in increased average pain relief (i.e., higher percent pain relief) compared to unimodal stimulation, which only provides motor or sensory signals.
wherein a system with two or more electrodes provides a coordinated multimodal (e.g., bimodal) signal to mimic the natural coordination between motor and sensory signals resulting in an increased likelihood of experiencing pain relief compared to unimodal stimulation, which only provides motor or sensory signals.
wherein a system with two or more electrodes provides a coordinated multimodal (e.g., bimodal) signal to mimic the natural coordination between motor and sensory signals resulting in a shorter duration of time prior to experiencing pain relief compared to unimodal stimulation, which only provides motor or sensory signals.
wherein a system with two or more electrodes provides a coordinated multimodal (e.g., bimodal) signal to mimic the natural coordination between motor and sensory signals resulting in an increased duration of pain relief compared to unimodal stimulation, which only provides motor or sensory signals.
wherein a system with two or more electrodes provides a coordinated multimodal signal to nerves that differ from sensory and motor nerves, such as, but not limited to, parasympathetic nerves and sympathetic nerves.
wherein the first electrode delivers stimulation to activate large diameter primary afferent fibers.
wherein the second electrode delivers stimulation to activate large diameter efferent motor fibers.
wherein the second electrode delivers stimulation to generate indirect proprioceptive afferent signals (reafferent signals).
wherein the afferent, efferent, and reafferent signals are coordinated into a composite signal.
wherein the combinatory afferent, efferent, and reafferent signals converge in order to mimic the natural coordination between sensory and motor signals in the central nervous system, provide biomimetic signals, or provide signals outside the realm of natural sensations to produce changes in central pain processing.
wherein the afferent, efferent, and reafferent signals are provides superior neural signaling to more effectively recondition the underlying pain state for superior relief.
wherein the afferent, efferent, and reafferent signals create complex, quasi-physiological (biomimetic) neural signals to more effectively reduce pain.
wherein the afferent, efferent, and reafferent signals stimulate an unhealthy nerve and/or nerve with damage or nerve with pre-existing damage.
wherein the coordination between the afferent, efferent, and reafferent signals are increase the likelihood of relief.
wherein the coordination between the afferent, efferent, and reafferent signals decrease the amount of time until there is relief.
wherein the coordination between the afferent, efferent, and reafferent signals increase the proportion of individuals with relief regardless of pain etiologies.
wherein the coordination between the afferent, efferent, and reafferent signals increase the duration of relief.
wherein the coordination between afferent, efferent, and reafferent signals avoid overlapping signals to avoid superposition of signals.
wherein the coordination between afferent, efferent, and reafferent signals avoid overlapping signals to avoid muscle fatigue.
wherein the coordination between afferent, efferent, and reafferent signals avoid overlapping signals to avoid nerve fatigue.
wherein the coordination between afferent, efferent, and reafferent signals avoid overlapping signals to avoid tissue damage.
wherein the coordination between afferent, efferent, and reafferent signals avoid overlapping signals to avoid painful stimulation.
wherein the coordination between afferent, efferent, and reafferent signals avoid overlapping signals to avoid off-target activation.

Disclosed is a multimodal peripheral nerve stimulation system and method. The system and method may comprise placing one or more lead(s) with one or more electrode(s) into a portion of a body that is proximal to a region of pain. The system and method may comprise stimulating at least one nerve that innervates the region of pain through two or more electrodes formed integrally on one or more leads, wherein the electrodes are positioned inside the body and placed near at least one nerve or nerve branch that innervates the region(s) of pain. The system and method may comprise application of electrical stimulation with a set of stimulation parameters through the first electrode on the one or more leads, and electrical stimulation with an additional set of stimulation parameters through the one or more additional electrodes on the same of different lead, whereby the sets of stimulation parameters are provided in various fashions to produce the synergistic effect in order to provide pain relief to systems or regions disrupted by disease, trauma, overuse, or otherwise subject to maladaptive changes.

The system and method may comprise one or more (or all) of any of the following, in any combination:
wherein a system for applying electrical stimulation via two or more electrodes of differing frequencies (e.g., multiple modes, multimodal, bimodal) may be used in order to recondition the centrally maintained pain state.
wherein the two or more frequencies provided over two or more electrodes may be used to activate neural fibers that engage multiple mechanisms to provide pain relief, resulting in a synergistic and/or complementary effect on neural pathways and central pain processing, and more comprehensive relief (e.g., providing pain relief of one or multiple etiologies of pain in one or multiple locations of pain).
wherein one or more electrodes that provide stimulation at lower frequency(ies) (e.g., 4-20 Hz) may be used to target motor nerve fibers (e.g., motor stimulation) in a motor nerve or mixed (e.g., motor and sensory) nerve resulting in cyclical muscle twitches and/or muscle contractions and indirectly activate proprioceptive afferent pathways.
wherein a periodic, cyclical, cycled, intermittent, and/or continuous motor nerve response elicits a comfortable muscle twitch and/or contraction without unwanted fatigue.
wherein one or more electrodes may be used to provide stimulation at higher frequency(ies) (e.g., 60-120 Hz) to activate directly afferent pathways (e.g., sensory stimulation).
wherein sensory nerve responses provide a continuous paresthesia over the region of pain.
wherein two or more electrodes may be placed near the same nerve or nerve branch, engaging in both indirect and direct afferent pathways via combinatory motor stimulation and sensory stimulation.
wherein two or more electrodes may be placed near two or more nerves or nerve branches, engaging in indirect and/or direct afferent pathways via combinatory motor stimulation and sensory stimulation.
wherein two or more electrodes may be placed near one or more nerves or nerve branches, engaging in indirect and/or direct afferent pathways via combinatory motor stimulation and sensory stimulation.
wherein one or more electrodes provides a stimulation frequency low enough (e.g., 0.5-12 Hz or 0.1-20 Hz, or 0.05-40 Hz) to evoke visible muscle twitches (i.e. non-fused muscle contraction) and/or muscle contraction(s) of the targeted muscle(s) innervated by the target nerve or nerve branch, with or without a modulating envelope.
wherein one or more electrodes provides a stimulation frequency high enough (e.g. 20-120 Hz or 12-200 Hz or 200-500 Hz) to evoke sensation(s) or comfortable paresthesia(s) in the region(s) of pain or in alternate target region(s), with or without a modulating envelope.
wherein one or more electrodes provides a stimulation frequency high enough (e.g., 500-15000 Hz) to produce neural effect(s) that may be sub-perception in the region(s) of pain or in alternate target region(s), with or without a modulating frequency.
wherein a system contains more than one electrode and provides a combination of stimulation frequencies that evoke muscle twitches and/or muscle contractions in the region of pain, and/or evoke sensations or comfortable paresthesia(s) in the region of pain, and/or produce neural effects that may be sub-perception in the region of pain.
wherein a system may apply electrical stimulation wherein higher frequencies (e.g. 20-120 Hz or 12-200 Hz or 200-500 Hz or 500-1500 Hz) are applied intermittently (e.g., with a duty cycle less than 100%) at lower frequencies to produce patient sensation and muscle twitch without muscle fatigue.
wherein a system applies electrical motor stimulation at the cervical medial branch of the dorsal ramus and sensory stimulation at the occipital nerve for the treatment of pain in the head and neck.
wherein a system applies electrical motor stimulation at the lumbar medial branch of the dorsal ramus and sensory stimulation at the spinal nerve for the treatment of pain in the back and legs.
wherein a system applies electrical motor stimulation at the lumbar medial branch of the dorsal ramus and sub-perception stimulation at the lumbar medial branch of the dorsal ramus for the treatment of pain in the back and legs.
wherein a system applies motor stimulation at the axillary nerve at the deltoid and sensory stimulation at the suprascapular nerve for the treatment of pain in the shoulder.
wherein a system motor stimulation at the femoral nerve more distally (e.g., at the vastus medialis oblique) and sensory stimulation at the femoral nerve more proximally (e.g., just distal to the inguinal crease) for the treatment of pain in the knee.
wherein a system with two or more electrodes provides a coordinated multimodal (e.g., bimodal) signal to mimic the natural coordination between motor and sensory signals resulting in increased average pain relief (i.e., higher percent pain relief) compared to unimodal stimulation, which only provides motor or sensory signals.
wherein a system with two or more electrodes provides a coordinated multimodal (e.g., bimodal) signal to mimic the natural coordination between motor and sensory signals resulting in an increased likelihood of experiencing pain relief compared to unimodal stimulation, which only provides motor or sensory signals.
wherein a system with two or more electrodes provides a coordinated multimodal (e.g., bimodal) signal to mimic the natural coordination between motor and sensory signals resulting in a shorter duration of time prior to experiencing pain relief compared to unimodal stimulation, which only provides motor or sensory signals.
wherein a system with two or more electrodes provides a coordinated multimodal (e.g., bimodal) signal to mimic the natural coordination between motor and sensory signals resulting in an increased duration of pain relief compared to unimodal stimulation, which only provides motor or sensory signals.
wherein a system with two or more electrodes provides a coordinated multimodal signal to nerves that differ from sensory and motor nerves, such as, but not limited to, parasympathetic nerves and sympathetic nerves.
wherein the first electrode delivers stimulation to activate large diameter primary afferent fibers.
wherein the second electrode delivers stimulation to activate large diameter efferent motor fibers.
wherein the second electrode delivers stimulation to generate indirect proprioceptive afferent signals (reafferent signals).
wherein the afferent, efferent, and reafferent signals are coordinated into a composite signal.
wherein the combinatory afferent, efferent, and reafferent signals converge in order to mimic the natural coordination between sensory and motor signals in the central nervous system, provide biomimetic signals, or provide signals outside the realm of natural sensations to produce changes in central pain processing.
wherein the afferent, efferent, and reafferent signals are provides superior neural signaling to more effectively recondition the underlying pain state for superior relief.
wherein the afferent, efferent, and reafferent signals create complex, quasi-physiological (biomimetic) neural signals to more effectively reduce pain.
wherein the afferent, efferent, and reafferent signals stimulate an unhealthy nerve and/or nerve with damage or nerve with pre-existing damage.
wherein the coordination between the afferent, efferent, and reafferent signals are increase the likelihood of relief.
wherein the coordination between the afferent, efferent, and reafferent signals decrease the amount of time until there is relief.
wherein the coordination between the afferent, efferent, and reafferent signals increase the proportion of individuals with relief regardless of pain etiologies.
wherein the coordination between the afferent, efferent, and reafferent signals increase the duration of relief.
wherein the coordination between afferent, efferent, and reafferent signals avoid overlapping signals to avoid superposition of signals.
wherein the coordination between afferent, efferent, and reafferent signals avoid overlapping signals to avoid muscle fatigue.
wherein the coordination between afferent, efferent, and reafferent signals avoid overlapping signals to avoid nerve fatigue.
wherein the coordination between afferent, efferent, and reafferent signals avoid overlapping signals to avoid tissue damage.
wherein the coordination between afferent, efferent, and reafferent signals avoid overlapping signals to avoid painful stimulation.
wherein the coordination between afferent, efferent, and reafferent signals avoid overlapping signals to avoid off-target activation.

The following description and the drawings disclose various illustrative aspects. Some improvements and novel aspects may be expressly identified, while others may be apparent from the description and drawings.

### DESCRIPTION OF THE DRAWINGS

Operation of the present teachings may be better understood by reference to the detailed description taken in connection with the following illustrations. These appended drawings form part of this specification, and written information in the drawings should be treated as part of this disclosure. In the drawings:

FIG. 1 illustrates an embodiment of a system and method for applying multimodal (e.g., bimodal) stimulation with at least two leads.

FIGS. 2A and 2B are schematic anatomic views, respectively anterior and lateral, of a human peripheral nervous system.

FIG. 3A is a schematic anatomic view of a human spine, showing the various regions and the vertebrae comprising the regions.

FIGS. 3B and 3C are schematic anatomic views of the dermatome boundaries of a human.

FIGS. 4A, 4B, and 4C are anatomic views of the intercostal spinal nerves of a human.

FIGS. 5A and 5B are anatomic views of the spinal nerves of the brachial plexus.

FIG. 6 is an anatomic view of the spinal nerves of the lumbar plexus.

FIG. 7 is an anatomic view of the spinal nerves of the sacral plexus.

FIG. 8 is an anatomic view of the spinal nerves of the cervical plexus.

FIG. 9 is an anatomic view of the spinal nerves of the solar plexus.

FIGS. 10A, 10B, and 10C are schematic sectional anatomic views of a system for applying stimulation along a sciatic/tibial nerve at multiple locations.

FIGS. 11 and 12 are an idealized, diagrammatic view showing a nerve, branch of nerve, or motor stimulation system.

FIG. 13A-D illustrates exemplary outcomes after a temporary treatment from systems providing unimodal stimulation (unimodal: sensory or unimodal: motor) versus multimodal (e.g., bimodal) stimulation.

FIG. 14A-D illustrates exemplary outcomes after a temporary treatment from systems providing unimodal motor stimulation or unimodal sensory stimulation versus multimodal (e.g., bimodal) stimulation.

FIG. 15A-D illustrates exemplary outcomes after a temporary treatment from systems providing unimodal X stimulation or unimodal X stimulation versus multimodal (e.g., bimodal) stimulation.

FIG. 16A-D illustrates exemplary outcomes after a temporary stimulation delivery period with multimodal (e.g., bimodal) stimulation in patients with unhealthy nerves and/or nerve damage or pre-existing nerve damage.

FIG. 17A-C illustrates exemplary outcomes after a permanently implanted stimulation system provides unimodal motor stimulation or unimodal sensory stimulation versus multimodal (e.g., bimodal) stimulation.

FIG. 18A-D is a schematic view of the shoulder anatomy showing example nerve targets for a system to apply (A) motor stimulation (e.g., cyclic pulses at 12 Hz) to the axillary nerve at the deltoid and (B) sensory stimulation (e.g., continuous pulses at 100 Hz) to the suprascapular nerve and/or axillary nerves for the treatment of shoulder pain.

FIG. 19 is a schematic view of the back of the neck showing example nerve targets for a system to apply motor stimulation and sensory stimulation for the treatment of pain in the head and neck.

FIG. 20 is a schematic view of the lumbar neural anatomy showing example nerve targets for a system to apply motor stimulation and sensory stimulation for the treatment of pain in the back and legs.

FIGS. 21A-D are views showing percutaneous lead(s) that can form a part of a nerve, branch of nerve, or muscle stimulation system.

FIG. 22 is a view of a package containing a nerve, branch of nerve, or muscle stimulation system with multiple percutaneous leads.

FIGS. 23A-B are representative leads that can form a part of a nerve, branch of nerve, or muscle stimulation system.

FIGS. 24A-B are representative leads that can form a part of a nerve, branch of nerve, or muscle stimulation system.

FIGS. 25A-B are schematic anatomic views of a system for applying stimulation to spinal nerves in the brachial plexus and the axillary nerve.

FIGS. 26A-C are schematic anatomic views of a system for applying stimulation to a femoral nerve or branch at multiple locations.

FIGS. 27A-C are schematic anatomic views of a system for applying stimulation to a sciatic/tibial nerve at multiple locations.

FIGS. 28A-B are schematic sectional anatomic views of systems for applying stimulation to a femoral nerve and a sciatic/tibial nerve at multiple locations.

FIG. 29A-C illustrate non-exhaustive harmonic frequencies designed to provide stimulation at more than one frequency across more than one electrode.

FIG. 30A is a schematic view showing an example for a system to apply unimodal stimulation (e.g., sensory stimulation alone or motor stimulation alone) from one electrode to provide stimulation to one nerve or nerve branch target.

FIG. 30B is a schematic view showing an example for a system to apply unimodal stimulation (e.g., sensory stimulation alone or motor simulation alone) from two leads, each with one electrode, to provide stimulation to two nerve or nerve branch targets.

FIG. 31A is a schematic view showing non-limiting example nerve targets for a system to apply multimodal stimulation (e.g., motor stimulation and sensory stimulation) for the treatment of pain.

FIG. 31B is a schematic view showing different example regions of pain relief provided by the example multimodal (e.g., bimodal) nerve targets illustrated in FIG. 31A.

FIG. 32A-C illustrate peripheral nerve stimulation approaches designed to selectively activate large diameter nerve fibers, where the dotted circles around the electrodes represent stimulation waveforms or the area or zone of activation (e.g., area that is being activated).

FIG. 33A-C illustrate example signal propagation for afferent, efferent, and mixed nerves for a system providing unimodal sensory stimulation, unimodal motor stimulation, or multimodal (e.g., bimodal) stimulation.

FIG. 34A illustrates an exemplary system providing unimodal sensory stimulation.

FIG. 34B-D illustrates an exemplary system providing bimodal (or multimodal) stimulation.

FIG. 35A-C are non-limiting examples of multimodal (e.g., bimodal) applications of motor and sensory stimulation.

FIG. 36A-F are non-limiting examples coordinated multimodal (e.g., bimodal) stimulation targets to treat various regions of pain throughout the body.

FIG. 37A-C illustrates the responder rate (proportion of patients with greater than equal to 50% pain relief) among patients who received treatment with either unimodal stimulation (unimodal: sensory or unimodal: motor) or bimodal stimulation.

FIG. 38A-B represent data collected from multiple medical centers among patients who underwent peripheral nerve stimulation treatment for shoulder pain.

FIG. 39 illustrates results of an analysis of average patient-reported pain relief among responders.

FIG. 40A-L illustrate non-exhaustive examples of methods for applying multimodal stimulation to one or more nerves via one or more leads with one or more electrodes.

FIG. 41A-B illustrate exemplary unimodal stimulation amplitudes and stimulation pulse patterns.

FIG. 41C-E illustrate non-exhaustive, combinatory stimulation amplitudes and stimulation pulse patterns that can be applied to one or more electrodes.

FIG. 42 illustrates exemplary outcomes after a temporary treatment from systems providing unimodal X stimulation or unimodal X stimulation versus multimodal (e.g., bimodal) stimulation.

FIG. 43A-C illustrate exemplary unimodal stimulation amplitudes and patterns.

FIG. 43D illustrates a non-exhaustive, combinatory stimulation (Multimodal Waveform XYZa) amplitude and/or intensity and/or pattern with two continuous (e.g., tonic) stimulation patterns applied at different frequencies, and these patterns may be applied through the same or more than one electrode.

FIG. 44A-C is an illustrative example of peripheral nerve fiber activation following stimulation (e.g., sensory nerve fiber stimulation) for the treatment of pain.

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DETAILED DESCRIPTION

Reference will now be made in detail to exemplary embodiments of the present teachings, examples of which are illustrated in the accompanying drawings. It is to be understood that other embodiments may be utilized and structural and functional changes may be made without departing from the respective scope of the present teachings. Moreover, features of the various embodiments may be combined or altered without departing from the scope of the present teachings. As such, the following description is presented by way of illustration only and should not limit in any way the various alternatives and modifications that may be made to the illustrated embodiments and still be within the spirit and scope of the present teachings.

As used herein, the words “example” and “exemplary” mean an instance, or illustration. The words “example” or “exemplary” do not indicate a key or preferred aspect or embodiment. The word “or” is intended to be inclusive rather an exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more” unless context suggest otherwise.

The described configurations, elements or complete assemblies and methods and their elements for carrying out the disclosed system and method, and variations of aspects of the disclosed system and method can be combined and modified with each other in any combination.

Generally, disclosed are systems and methods that provide bimodal or multimodal electrical stimulation at one or more nerve or peripheral nerve sites to provide pain relief, in an example. As described herein, the systems and methods may include at least two electrodes. It is noted that the systems and methods may include any number greater than one electrode, e.g., 2, 3, 4, 5, 6, 7, 8, 9, 10, etc. electrodes. The at least two electrodes may provide the same electrical stimulation or different electrical stimulation (e.g., one or more (or all) of time and duration of electrical stimulation, frequency, pulse duration, amplitude, duty cycle, pattern of stimulus pulses, polarity, a predetermined number of phases, waveform shape, and the like). The at least two electrodes may be provided on any number of leads, e.g., 1, 2, 3, 4, 5, 6, 7, 8, 9, 10, etc. leads, and the leads may be used with any number of stimulators, e.g., 1, 2, 3, 4, 5, 6, 7, 8, 9, 10, etc. stimulators.

In an embodiment, the systems and methods may include the same number of electrodes and leads (with a different, e.g., smaller, number of stimulators); the same number of leads and stimulators (with a different, e.g., greater, number of electrodes); the same number of electrodes, leads, and stimulators; and the like. In an embodiment, the systems and methods may include a different number for each of electrodes, leads, and stimulators. In an embodiment, the systems and methods may include a greater number of electrodes than leads. In an embodiment, the systems and methods may include a greater number of electrodes than stimulators. In an embodiment, the systems and methods may include a greater number of leads than stimulators.

In an embodiment, the at least two electrodes may be provided on a single lead. In an embodiment, the at least two electrodes may be provided on more than one lead. In an embodiment, the systems and methods may include at least one lead. In an embodiment, the systems and methods may include at least two leads. For example, a first lead may comprise a first electrode and a second lead may comprise a second electrode. Each the first and/or the second lead may also comprise more than one electrode. The at least two electrodes may provide the same electrical stimulation or different electrical stimulation (e.g., one or more (or all) of time and duration of electrical stimulation, frequency, pulse duration, amplitude, duty cycle, pattern of stimulus pulses, polarity, a predetermined number of phases, waveform shape, and the like).

In an embodiment, the at least two leads may be provided on a single stimulator. In an embodiment, the at least two leads may be provided on more than one stimulator. In an embodiment, the systems and methods may include at least one stimulator. In an embodiment, the systems and methods may include at least two stimulators. For example, a first lead having at least one electrode may be provided on a first stimulator and a second lead having at least one electrode may be provided on a second stimulator. It is noted that both the first and the second lead may also be provided on the same stimulator and such stimulator may comprise two or more channels that the leads may be coupled to. In embodiments having more than one stimulator, all stimulators may be implantable, all stimulators may be external, or the stimulators may be a combination of implantable and external stimulators. For example, a first lead having at least one electrode may be provided on a first implantable stimulator and a second lead having at least one electrode may be provided on a second external stimulator. It is noted that each stimulator may have more than one lead and each lead may have more than one electrode (so that the system and methods have at least two electrodes). The at least two electrodes may provide the same electrical stimulation or different electrical stimulation (e.g., one or more (or all) of time and duration of electrical stimulation, frequency, pulse duration, amplitude, duty cycle, pattern of stimulus pulses, polarity, a predetermined number of phases, waveform shape, and the like).

### I. The Peripheral Nervous System (Anatomic Overview)

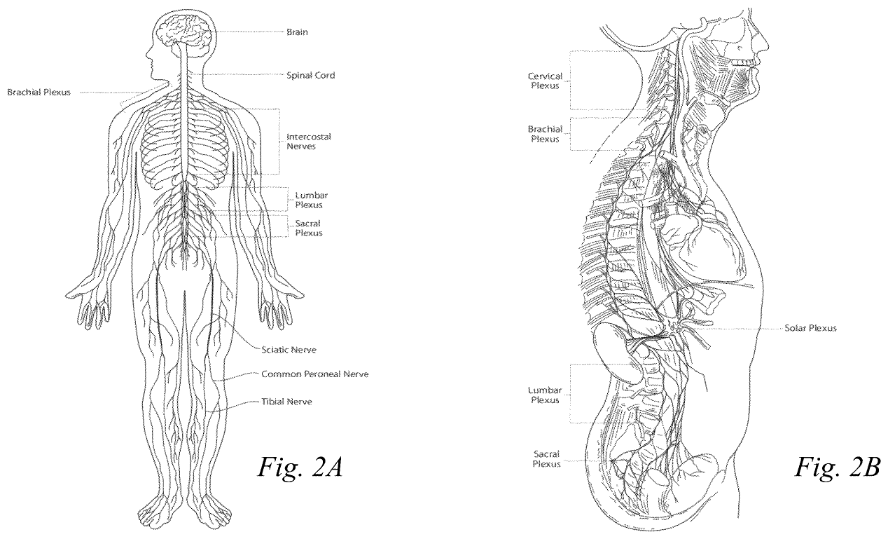

As generally shown in FIGS. 2A and 2B, the peripheral nervous system consists of nerve fibers and cell bodies outside the central nervous system (the brain and the spinal column) that conduct impulses to or away from the central nervous system. The peripheral nervous system is made up of nerves (called spinal nerves) that connect the central nervous system with peripheral structures. The nerves of the peripheral nervous system arise from the spinal column and exit through intervertebral foramina in the vertebral column (spine). The afferent, or sensory, fibers of the peripheral nervous system convey neural impulses to the central nervous system from the sense organs (e.g., the eyes) and from sensory receptors in various parts of the body (e.g., the skin, muscles, etc.). The efferent, or motor, fibers convey neural impulses from the central nervous system to the effector organs (muscles and glands).

The somatic nervous system (SNS) is the part of the peripheral nervous system associated with the voluntary control of body movements through the action of skeletal muscles, and with reception of external stimuli, which helps keep the body in touch with its surroundings (e.g., touch, hearing, and sight). The system includes all the neurons connected with skeletal muscles, skin and sense organs. The somatic nervous system consists of efferent nerves responsible for sending central nervous signals for muscle contraction. A somatic nerve is a nerve of the somatic nervous system.

### A. Nerves

A typical nerve in the peripheral nervous system arises from the spinal cord by rootlets which converge to form two nerve roots, the dorsal (sensory) root and the ventral (motor) root. The dorsal and ventral roots unite into a mixed nerve trunk that divides into a smaller dorsal (posterior) primary ramus and a much larger ventral (anterior) primary ramus. The posterior primary rami serve a column of muscles on either side of the vertebral column, and a narrow strip of overlying skin. All of the other muscle and skin is supplied by the anterior primary rami.

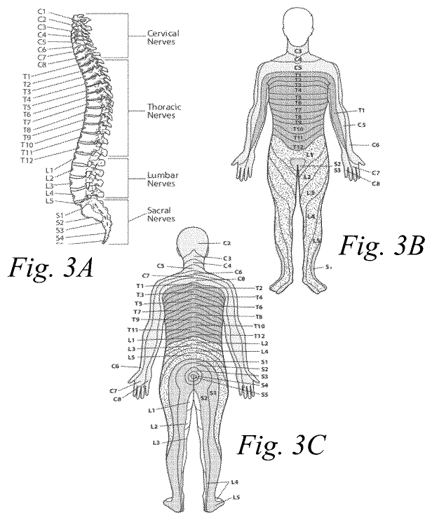

The nerve roots that supply or turn into peripheral nerves can be generally categorized by the location on the spine where the roots exit the spinal cord, e.g., as generally shown in FIG. 3A, cervical (generally in the head/neck, designated C1 to C8), thoracic (generally in chest/upper back, designated T1 to T12), lumbar (generally in lower back, designated L1 to L5); and sacral (generally in the pelvis, designated S1 to S5). All peripheral nerves can be traced back (toward the spinal column) to one or more of the nerve roots in either the cervical, thoracic, lumbar, or sacral regions of the spine. The neural impulses comprising pain felt in a given muscle or cutaneous region of the body pass through nerves and (usually) one or more nerve plexuses. For this reason, the nerves of the peripheral nervous system will sometimes be called in shorthand for the purpose of description “nerves of passage.” The nerves begin as roots at the spine, and can form trunks that divide by divisions or cords into branches that innervate skin and muscles.

Peripheral nerves have motor fibers and sensory fibers. The motor fibers innervate certain muscles, while the sensory fibers innervate any structure or tissue that has sensation, which may include muscle. A skin area innervated by the sensory fibers of a single nerve root is known as a dermatome. A group of muscles primarily innervated by the motor fibers of a single nerve root is known as a myotome. Although slight variations do exist, dermatome and myotome patterns of distribution are relatively consistent from person to person. It is to be understood that, although muscles and skin are discussed as examples, nerves also innervate other nearby structures such as joints, bones, adipose tissue, connective tissue, etc. For example, portions or branches of the sciatic and femoral nerves do not innervate only the muscles and skin of the lower extremity, they also innervate the bone, joints, adipose tissue, connective tissue, etc., and treatment of pain therein is also contemplated hereby.

Each muscle in the body is supplied by a particular level or segment of the spinal cord and by its corresponding peripheral nerve. The muscle, and its nerve make up a myotome. This is approximately the same for every person and are as follows:

C3,4 and 5 supply the diaphragm (the large muscle between the chest and the belly that we use to breath). C5 also supplies the shoulder muscles and the muscle that we use to bend our elbow. C6 is for bending the wrist back. C7 is for straightening the elbow. C8 bends the fingers. T1 spreads the fingers. T1-T12 supplies the chest wall & abdominal muscles. L2 bends the hip. L3 straightens the knee. L4 pulls the foot up. L5 wiggles the toes. S1 pulls the foot down. S3, S4 and S5 supply the bladder, bowel, and sex organs and the anal and other pelvic muscles.

Dermatome is a Greek word which literally means “skin cutting”. A dermatome is an area of the skin supplied by nerve fibers originating from the dorsal nerve root(s). The dermatomes are named according to the peripheral nerve which supplies them. The dermatomes form into bands around the trunk (see FIGS. 3B and 3C), but in the limbs their organization can be more complex as a result of the dermatomes being “pulled out” as the limb buds form and develop into the limbs during embryological development.

In the diagrams or maps shown in FIGS. 3B and 3C, the boundaries of dermatomes are usually sharply defined. However, in life there is considerable overlap of innervation between adjacent dermatomes. Thus, if there is a loss of afferent nerve function by one nerve sensation from the region of skin which it supplies is not usually completely lost as overlap from adjacent nerves occurs; however, there may be a reduction in sensitivity.

### B. Intercostal Nerves

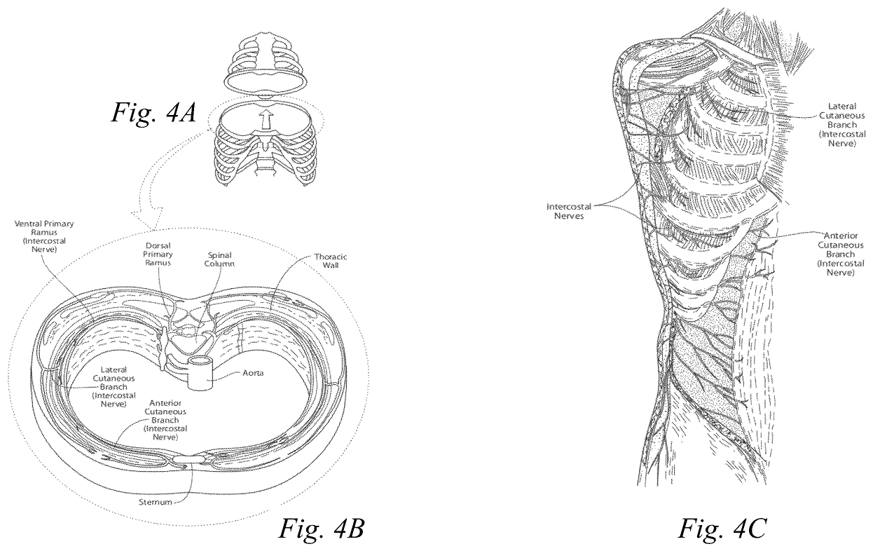

The intercostal nerves (see FIGS. 4A, 4B, and 4C) are the anterior divisions of thoracic nerves from the thoracic vertebrae T1 to T11. The intercostal nerves are distributed chiefly to the thoracic pleura and abdominal peritoneum and differ from the anterior divisions of the other peripheral nerves in that each pursues an independent course without plexus formation.

The first two nerves supply fibers to the upper limb in addition to their thoracic branches; the next four are limited in their distribution to the parietes of the thorax; the lower five supply the parietes of the thorax and abdomen. The 7th intercostal nerve terminates at the xyphoid process, at the lower end of the sternum. The 10th intercostal nerve terminates at the umbilicus. The twelfth (subcostal) thoracic is distributed to the abdominal wall and groin.

Branches of a typical intercostal nerve include the ventral primary ramus; lateral cutaneous branches that pass beyond the angles of the rubs and innervate the internal and external intercostal muscles approximately halfway around the thorax; and the anterior cutaneous branches that supply the skin on the anterior aspect of the thorax and abdomen.

### C. Nerve Plexuses

A nerve plexus is a network of intersecting anterior primary rami. The sets of anterior primary rami form nerve trunks that ultimately further divide through divisions and then into cords and then into nerve branches serving the same area of the body. The nerve branches are mixed, i.e., they carry both motor and sensory fibers. The branches innervate the skin, muscle, or other structures. One example of the entry of a terminal motor nerve branch into muscle is called a motor point.

As shown in FIGS. 2A and 2B, there are several nerve plexuses in the body, including (i) the brachial plexus, which serves the chest, shoulders, arms and hands; (ii) the lumbar plexus, which serves the back, abdomen, groin, thighs, knees, and calves; (iii) the sacral plexus, which serves the buttocks, thighs, calves, and feet; (iv) the cervical plexus, which serves the head, neck and shoulders; and (vi) the solar plexus, which serves internal organs. The following describes, from an anatomic perspective, the nerves passing through the various plexuses, and the muscle and/or skin regions they innervate and where pain can be felt.

### 1. The Brachial Plexus

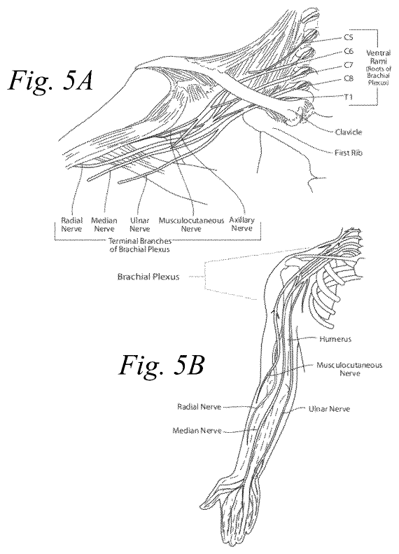

Most nerves in the upper limb arise from the brachial plexus, as shown in FIGS. 5A and 5B. The brachial plexus begins in the neck (vertebrae C5 through C7), forms trunks, and extends through divisions and cords into the axilla (underarm), where nearly all the nerve branches arise. Primary nerve branches of the brachial plexus include the musculocutaneous nerve; the median nerve; the ulnar nerve; the axillary nerve; and the radial nerve.

a. The Musculocutaneous Nerve

The musculocutaneous nerve arises from the lateral cord of the brachial plexus. Its fibers are derived from cervical vertebrae C5, C6. The musculocutaneous nerve penetrates the coracobrachialis muscle and passes obliquely between the biceps brachii and the brachialis, to the lateral side of the arm. Just above the elbow, the musculocutaneous nerve pierces the deep fascia lateral to the tendon of the biceps brachii continues into the forearm as the lateral antebrachial cutaneous nerve. In its course through the arm, the musculocutaneous nerve innervates the coracobrachialis, biceps brachii, and the greater part of the brachialis.

b. The Median Nerve

The median nerve is formed from parts of the medial and lateral cords of the brachial plexus, and continues down the arm to enter the forearm with the brachial artery. It originates from the brachial plexus with roots from cervical vertebrae C5, C6, C7 and thoracic vertebra T1. The median nerve innervates all of the flexors in the forearm, except flexor carpi ulnaris and that part of flexor digitorum profundus that supplies the medial two digits. The latter two muscles are supplied by the ulnar nerve of the brachial plexus. The median nerve is the only nerve that passes through the carpal tunnel, where it may be compressed to cause carpal tunnel syndrome.

The main portion of the median nerve supplies the following muscles: (i) the superficial group comprising pronator teres muscle; flexor carpi radialis muscle; palmaris longus muscle; and (ii) the intermediate group comprising flexor digitorum superficialis muscle.

The anterior interosseus branch of the median nerve supplies the deep group comprising flexor digitorum profundus muscle (lateral half); flexor pollicis longus muscle; and pronator quadratus.

In the hand, the median nerve supplies motor innervation to the 1st and 2nd lumbrical muscles. It also supplies the muscles of the thenar eminence by a recurrent thenar branch. The rest of the intrinsic muscles of the hand are supplied by the ulnar nerve of the brachial plexus.

The median nerve innervates the skin of the palmar side of the thumb, the index and middle finger, half the ring finger, and the nail bed of these fingers. The lateral part of the palm is supplied by the palmar cutaneous branch of the median nerve, which leaves the nerve proximal to the wrist creases. The palmar cutaneous branch travels in a separate fascial groove adjacent to the flexor carpi radialis and then superficial to the flexor retinaculum. It is therefore spared in carpal tunnel syndrome.

c. The Ulnar Nerve

The ulnar nerve comes from the medial cord of the brachial plexus, and descends on the posteromedial aspect of the humerus. It goes behind the medial epicondyle, through the cubital tunnel at the elbow (where it is vulnerable to injury for a few centimeters, just above the joint). One method of injuring the nerve is to strike the medial epicondyle of the humerus from posteriorly, or inferiorly with the elbow flexed. The ulnar nerve is trapped between the bone and the overlying skin at this point. This is commonly referred to as hitting one's “funny bone.”

The ulnar nerve is the largest nerve not protected by muscle or bone in the human body. The ulnar nerve is the only unprotected nerve that does not serve a purely sensory function. The ulnar nerve is directly connected to the little finger, and the adjacent half of the ring finger, supplying the palmar side of these fingers, including both front and back of the tips, as far back as the fingernail beds.

The ulnar nerve and its branches innervate muscles in the forearm and hand. In the forearm, the muscular branches of ulnar nerve innervates the flexor carpi ulnaris and the flexor digitorum profundus (medial half). In the hand, the deep branch of ulnar nerve innervates hypothenar muscles; opponens digiti minimi; abductor digiti minimi; flexor digiti minimi brevis; adductor pollicis; flexor pollicis brevis (deep head); the third and fourth lumbrical muscles; dorsal interossei; palmar interossei. In the hand, the superficial branch of ulnar nerve innervates palmaris brevis.

The ulnar nerve also provides sensory innervation to the fifth digit and the medial half of the fourth digit, and the corresponding part of the palm. The Palmar branch of ulnar nerve supplies cutaneous innervation to the anterior skin and nails. The dorsal branch of ulnar nerve supplies cutaneous innervation to the posterior skin (except the nails).

d. The Axillary Nerve

The axillary nerve comes off the posterior cord of the brachial plexus at the level of the axilla (armpit) and carries nerve fibers from vertebrae C5 and C6. The axillary nerve travels through the quadrangular space with the posterior circumflex humeral artery and vein. It supplies two muscles: the deltoid (a muscle of the shoulder), and the teres minor (one of the rotator cuff muscles). The axillary nerve also carries sensory information from the shoulder joint, as well as from the skin covering the inferior region of the deltoid muscle, i.e., the “regimental badge” area (which is innervated by the superior lateral cutaneous nerve branch of the axillary nerve). When the axillary nerve splits off from the posterior cord, the continuation of the cord is the radial nerve.

e. The Radial Nerve

The radial nerve supplies the upper limb, supplying the triceps brachii muscle of the arm, as well as all twelve muscles in the posterior osteofascial compartment of the forearm, as well as the associated joints and overlying skin. The radial nerve originates from the posterior cord of the brachial plexus with roots from cervical vertebrae C5, C6, C7, C8 and thoracic vertebra T1.

Cutaneous innervation is provided by the following nerves: (i) posterior cutaneous nerve of arm (originates in axilla); (ii) inferior lateral cutaneous nerve of arm (originates in arm); and (iii) posterior cutaneous nerve of forearm (originates in arm). The superficial branch of the radial nerve provides sensory innervation to much of the back of the hand, including the web of skin between the thumb and index finger.

Muscular branches of the radial nerve innervate the triceps brachii; anconeus brachioradialis; and the extensor carpi radialis longus.

The deep branch of the radial nerve innervates the extensor carpi radialis brevis; supinator; posterior interosseous nerve (a continuation of the deep branch after the supinator): extensor digitorum; extensor digiti minimi; extensor carpi ulnaris; abductor pollicis longus; extensor pollicis brevis; extensor pollicis longus; and extensor indicis.

The radial nerve (and its deep branch) provides motor innervation to the muscles in the posterior compartment of the arm and forearm, which are mostly extensors.

### 2. Sacral and Lumbar Plexuses

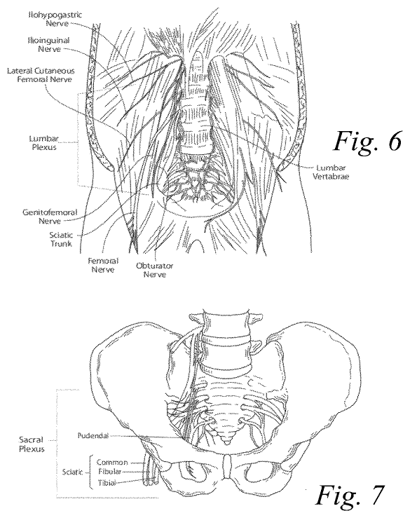

The lumbar plexus (see FIG. 6) is a nervous plexus in the lumbar region of the body and forms part of the lumbosacral plexus. It is formed by the ventral divisions of the first four lumbar nerves (L1-L4) and from contributions of the subcostal thoracic nerve (T12), which is the last (most inferior) thoracic nerve.

Additionally, the ventral rami of sacral vertebrae S2 and S3 nerves emerge between digitations of the piriformis and coccygeus nuscles. The descending part of the lumbar vertebrae L4 nerve unites with the ventral ramus of the lumbar vertebrae L5 nerve to form a thick, cordlike lumbosacral trunk. The lumbosacral trunk joins the sacral plexus (see FIG. 7). The main nerves of the lower limbs arise from the lumbar and sacral plexuses.

a. Nerves of the Sacral Plexus

The sacral plexus provides motor and sensory nerves for the posterior thigh, most of the lower leg, and the entire foot.

### (1) The Sciatic Nerve

As shown in FIGS. 2A and 7, the sciatic nerve (also known as the ischiatic nerve) arises from the sacral plexus. It is the longest and widest single nerve in the human body. It begins in the lower back and runs through the buttock and down the lower limb. The sciatic nerve supplies nearly the whole of the skin of the leg, the muscles of the back of the thigh, and those of the leg and foot. It is derived from spinal nerves L4 through S3. It contains fibers from both the anterior and posterior divisions of the lumbosacral plexus.

The nerve gives off articular and muscular branches. The articular branches (rami articulares) arise from the upper part of the nerve and supply the hip-joint, perforating the posterior part of its capsule; they are sometimes derived from the sacral plexus. The muscular branches (rami musculares) innervate the following muscles of the lower limb: biceps femoris, semitendinosus, semimembranosus, and adductor magnus. The nerve to the short head of the biceps femoris comes from the common peroneal part of the sciatic, while the other muscular branches arise from the tibial portion, as may be seen in those cases where there is a high division of the sciatic nerve.

The muscular branch of the sciatic nerve eventually gives off the tibial nerve (shown in FIG. 2A) and common peroneal nerve (also shown in FIG. 2A), which innervates the muscles of the (lower) leg. The tibial nerve goes on to innervate all muscles of the foot except the extensor digitorum brevis (which is innervated by the peroneal nerve).

Two major branches of the sciatic nerve are the tibial and common peroneal nerves that innervate much of the lower leg (around and below the knee). For example, the tibial nerve innervates the gastrocnemius, popliteus, soleus and plantaris muscles and the knee joint. Most of the foot is innervated by the tibial and peroneal nerve.

b. Nerves of the Lumbar Plexus

The lumbar plexus (see FIG. 6) provides motor, sensory, and autonomic fibres to gluteal and inguinal regions and to the lower extremities. The gluteal muscles are the three muscles that make up the buttocks: the gluteus maximus muscle, gluteus medius muscle and gluteus minimus muscle. The inguinal region is situated in the groin or in either of the lowest lateral regions of the abdomen.

(1) The Iliohypogastric Nerve: The iliohypogastric nerve (see FIG. 6) runs anterior to the psoas major on its proximal lateral border to run laterally and obliquely on the anterior side of quadratus lumborum. Lateral to this muscle, it pierces the transversus abdominis to run above the iliac crest between that muscle and abdominal internal oblique. It gives off several motor branches to these muscles and a sensory branch to the skin of the lateral hip. Its terminal branch then runs parallel to the inguinal ligament to exit the aponeurosis of the abdominal external oblique above the external inguinal ring where it supplies the skin above the inguinal ligament (i.e. the hypogastric region) with the anterior cutaneous branch.

(2) The Ilioinguinal Nerve: The ilioinguinal nerve (see FIG. 6) closely follows the iliohypogastric nerve on the quadratus lumborum, but then passes below it to run at the level of the iliac crest. It pierces the lateral abdominal wall and runs medially at the level of the inguinal ligament where it supplies motor branches to both transversus abdominis and sensory branches through the external inguinal ring to the skin over the pubic symphysis and the lateral aspect of the labia majora or scrotum.

(3) The Genitofemoral Nerve: The genitofemoral nerve (see FIG. 6) pierces psoas major anteriorly below the former two nerves to immediately split into two branches that run downward on the anterior side of the muscle. The lateral femoral branch is purely sensory. It pierces the vascular lacuna near the saphenous hiatus and supplies the skin below the inguinal ligament (i.e. proximal, lateral aspect of femoral triangle). The genital branch differs in males and females. In males it runs in the spermatic cord and in females in the inguinal canal together with the teres uteri ligament. It then sends sensory branches to the scrotal skin in males and the labia majora in females. In males it supplies motor innervation to the cremaster.

(4) The Lateral Cutaneous Femoral Nerve: The lateral cutaneous femoral nerve (see FIG. 6) pierces psoas major on its lateral side and runs obliquely downward below the iliac fascia. Medial to the anterior superior iliac spine it leaves the pelvic area through the lateral muscular lacuna. In the thigh it briefly passes under the fascia lata before it breaches the fascia and supplies the skin of the anterior thigh.

(5) The Obturator Nerve: The obturator nerve (see FIG. 6) leaves the lumbar plexus and descends behind psoas major on it medial side, then follows the linea terminalis and exits through the obturator canal. In the thigh, it sends motor branches to obturator externus before dividing into an anterior and a posterior branch, both of which continues distally. These branches are separated by adductor brevis and supply all thigh adductors with motor innervation: pectineus, adductor longus, adductor brevis, adductor magnus, adductor minimus, and gracilis. The anterior branch contributes a terminal, sensory branch which passes along the anterior border of gracilis and supplies the skin on the medial, distal part of the thigh.

(6) The Femoral Nerve: The femoral nerve (see FIG. 6) is the largest and longest nerve of the lumbar plexus. It gives motor innervation to iliopsoas, pectineus, sartorius, and quadriceps femoris; and sensory innervation to the anterior thigh, posterior lower leg, and hindfoot. It runs in a groove between psoas major and iliacus giving off branches to both muscles. In the thigh it divides into numerous sensory and muscular branches and the saphenous nerve, its long sensory terminal branch which continues down to the foot.

The femoral nerve has anterior branches (intermediate cutaneous nerve and medial cutaneous nerve) and posterior branches. The saphenous nerve (branch of the femoral nerve) provides cutaneous (skin) sensation in the medial leg. Other branches of the femoral nerve innervate structures (such as muscles, joints, and other tissues) in the thigh and around the hip and knee joints. As an example, branches of the femoral nerve innervate the hip joint, knee joint, and the four parts of the Quadriceps femoris (muscle) Rectus femoris (in the middle of the thigh) originates on the ilium and covers most of the other three quadriceps muscles. Under (or deep to) the rectus femoris are the other 3 of the quadriceps muscles, which originate from the body of the femur. Vastus lateralis (on the outer side of the thigh) is on the lateral side of the femur. Vastus medialis (on the inner part thigh) is on the medial side of the femur. Vastus intermedius (on the top or front of the thigh) lies between vastus lateralis and vastus medialis on the front of the femur. Branches of the femoral nerve often innervate the pectineus and Sartorius muscles arises.

### 3. The Cervical Plexus

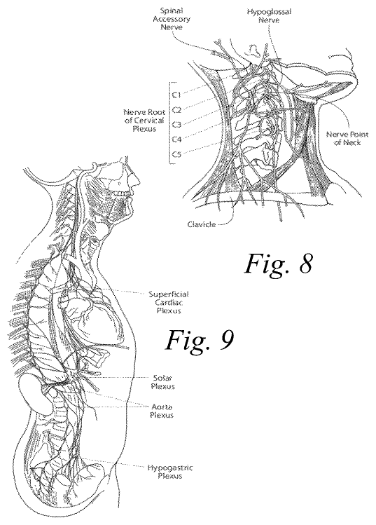

The cervical plexus (see FIG. 8) is a plexus of the ventral rami of the first four cervical spinal nerves which are located from C1 to C4 cervical segment in the neck. They are located laterally to the transverse processes between prevertebral muscles from the medial side and vertebral (m. scalenus, m. levator scapulae, m. splenius cervicis) from lateral side. Here there is anastomosis with accessory nerve, hypoglossal nerve and sympathetic trunk.

The cervical plexus is located in the neck, deep to sternocleidomastoideormed from the cervical plexus innervate the back of the head, as well as some neck muscles. The branches of the cervical plexus emerge from the posterior triangle at the nerve point, a point which lies midway on the posterior border of the sternocleidomastoid The nerves formed by the cervical plexus supply the back of the head, the neck and the shoulders. The face is supplied by a cranial nerve, the trigeminal nerve. The upper four posterior primary rami are larger than the anterior primary rami. The Cl posterior primary ramus does not usually supply the skin. The C2 posterior primary ramus forms the greater occipital nerve which supplies the posterior scalp. The upper four anterior primary rami form the cervical plexus. The cervical plexus supplies the skin over the anterior and lateral neck to just below the clavicle. The plexus also supplies the muscles of the neck including the scalenes, the strap muscles, and the diaphragm.

The cervical plexus has two types of branches: cutaneous and muscular. The cutaneous branches include the lesser occipital nerve, which innervates lateral part of occipital region (C2 nerve only); the great auricular nerve, which innervates skin near concha auricle and external acoustic meatus (C2 and C3 nerves); the transverse cervical nerve, which innervates anterior region of neck (C2 and C3 nerves); and the supraclavicular nerves, which innervate region of suprascapularis, shoulder, and upper thoracic region (C3, C4 Nerves). The muscular branches include the ansa cervicalis (loop formed from C1-C3), etc. (geniohyoid (C1 only), thyrohyoid (C1 only), sternothyroid, sternohyoid, omohyoid); phrenic (C3-C5 (primarily C4)), which innervates the diaphragm; the segmental branches (C1-C4), which innervate the anterior and middle scalenes.

### 4. The Solar Plexus

The solar plexus (see FIG. 9) is a dense cluster of nerve cells and supporting tissue, located behind the stomach in the region of the celiac artery just below the diaphragm. It is also known as the celiac plexus. Rich in ganglia and interconnected neurons, the solar plexus is the largest autonomic nerve center in the abdominal cavity. Through branches it controls many vital functions such as adrenal secretion and intestinal contraction.

Derived from the solar plexus are the phrenic plexus (producing contractions of the diaphragm, and providing sensory innervation for many components of the mediastinum and pleura); the renal plexuses (affecting renal function); the spermatic plexus (affecting function of the testis); as well as the gastric plexus; the hepatic plexus; the splenic plexus; the superior mesenteric plexus; and the aortic plexus.

### II. The System

Electrical stimulation systems hold promise for the relief of pain. Transcutaneous electrical nerve stimulation (TENS) has been cleared by the FDA for treatment of pain. TENS systems are external neurostimulation devices that use electrodes placed on the skin surface to activate target nerves below the skin surface. Application of TENS has been used to treat pain successfully, but it has low long-term patient compliance, because it may cause additional discomfort by generating cutaneous pain signals due to the electrical stimulation being applied through the skin, and the overall system is bulky, cumbersome, and not suited for long-term use.

In addition, several clinical and technical issues associated with surface electrical stimulation have prevented it from becoming a widely accepted treatment method. First, stimulation of cutaneous pain receptors cannot be avoided resulting in stimulation-induced pain that limits patient tolerance and compliance. Second, electrical stimulation is delivered at a relatively high frequency to prevent stimulation-induced pain, which leads to early onset of muscle fatigue in turn preventing patients from properly using their arm. Third, it is difficult to stimulate deep nerves and/or muscles with surface electrodes without stimulating overlying, more superficial nerves and/or muscles resulting in unwanted stimulation. Finally, clinical skill and intensive patient training is required to place surface electrodes reliably on a daily basis and adjust stimulation parameters to provide optimal treatment.

Spinal cord stimulation (SCS) systems are FDA approved as implantable neurostimulation devices marketed in the United States for treatment of pain. Similar to TENS, when SCS evokes paresthesias that cover the region of pain, it confirms that the location of the electrode and the stimulus intensity should be sufficient to provide pain relief and pain relief can be excellent initially, but maintaining sufficient paresthesia coverage is often a problem as the lead migrates along the spinal canal.

Spinal cord stimulation is limited by the invasive procedure and the decrease in efficacy as the lead migrates. When it can produce paresthesias in the region of pain, spinal cord stimulation is typically successful initially in reducing pain, but over time the paresthesia coverage and pain reduction is often lost as the lead migrates away from its target. Lead migration is the most common complication for spinal cord stimulators occurring in up to 45-88% of the cases. When the lead migrates, the active contact moves farther from the target fibers and loses the ability to generate paresthesias in the target area. SCS systems attempt to address this problem by using leads with multiple contacts so that as the lead travels, the next contact in line can be selected to be the active contact.

Dorsal root ganglion stimulation (DRGS) systems are FDA approved as implantable neurostimulation devices marketed in the United States for the treatment of pain. DRGS involves stimulating the dorsal root ganglion, which is a collection of nerve cell bodies outside of the epidural space in the spine. DRGS, as an example, involves implantation through or near the epidural space for thoracic and lumbar targets and a transforaminal approach for sacral targets. DRGS requires invasive surgery, which is technically challenging for implantation. DRGS leads are often guided through epidural space making proper placement near the dorsal root ganglion difficult, resulting in recruitment of off-target neural structures, for example but not limited to motor fibers, sensory fibers, nerves, subcutaneous tissue, and cells in the spinal cord that may generate undesirable (e.g., uncomfortable, painful) effects of stimulation.

Spinal cord stimulation systems stimulate the spinal cord and dorsal root ganglion stimulation system stimulate the dorsal root ganglion, whereas the present system and method involves peripheral nerve stimulation which stimulates the peripheral nerves, for example. SCS and DRGS systems may have leads with multiple, many, or more than one electrode and may have the capability of independently controlling each electrode (e.g., controlling the parameters of each electrode independently or controlling some or one of the paramters of each electrode independently). However, independent control of separate electrodes and/or the use of multiple electrodes may result in superposition of stimulation amplitudes and/or intensities and/or generate undesirable effects of stimulation (e.g., off-target activation, tissue damage, increased pain, additional pain, limited analgesic effect, muscle fatigue, nerve fatigue, cramping, and/or other negative outcomes of overstimulation or superposition). The present system and method overcomes issues with providing two or more modes (e.g., controlling two or more electrodes that are providing waveforms with one or more different parameters) by coordinating different modes (e.g., frequencies) in various fashions to produce the synergistic effect, for example, simultaneously, concurrently, interleaved, and/or in another pattern in order to mimic the natural coordination between signals in the central nervous system, provide biomimetic signals (e.g., signals that match, mimic, or resemble natural sensations), or provide signals outside the realm of natural sensations in order to provide pain relief to systems or regions disrupted by disease, trauma, overuse, or otherwise subject to maladaptive changes and to avoid undesirable effects of stimulation (e.g., off-target activation, tissue damage, increased pain, additional pain, limited analgesic effect, muscle fatigue, nerve fatigue, cramping, and/or other negative outcomes of overstimulation or superposition).

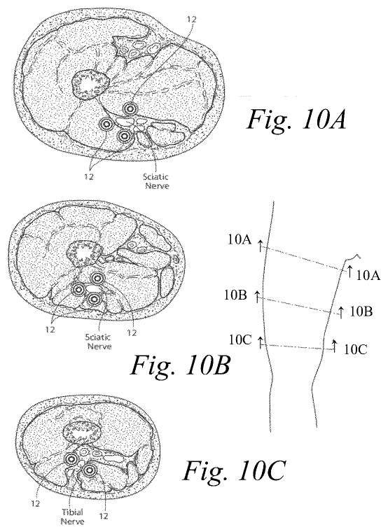

In various clinical applications of nerve stimulation to evoke sensory or muscle responses to treat pain or provide other therapeutic benefits, it may be desirable to stimulate different electrodes or leads at different frequencies. However, mixing two or more stimulus frequencies, in either percutaneous or fully implanted applications, can pose problems such as inconsistent and potentially problematic electrochemistry changes at the electrode(s) and inconsistent and potentially problematic electrochemistry changes at the anode or return electrode. Applying multiple frequencies of stimulation on multiple electrodes and/or leads can also pose an electronics design challenge, as this may require redundant components to generate stimulation signals in parallel, resulting in a larger size of an implanted pulse generator, which can present challenges for use by patients or surgical implantation, if the device is too large. In many embodiments, one system may provide stimulation via one mode (e.g., unimodal motor stimulation, unimodal sensory stimulation, or other unimodal stimulation such as but not limited to high frequency, low frequency, sub-threshold, or supra-threshold) to one electrode (FIG. 10A) or to more than one lead with one or more electrodes (FIG. 10B). The present embodiment overcomes the need for redundant circuitry by interleaving the stimulation modes (e.g., frequencies, patterns) on multiple channels using harmonic frequencies or patterns to enable a smaller, more efficient electronic device, suitable for use by patients and/or implantation.

In various clinical applications of nerve stimulation, unimodal stimulation (e.g., unimodal motor stimulation, unimodal sensory stimulation, or other unimodal stimulation such as but not limited to high frequency, low frequency, sub-threshold, or supra-threshold) provides a uniform signal to the central nervous system and lacks the complexity needed or the temporal patterning needed for engaging or maximizing analgesic mechanisms. Applying a uniform signal throughout therapy (e.g., via unimodal sensory stimulation) may lead to incomplete relief, minimal relief, no relief, and/or waning of pain relief over time (e.g., shorter lasting relief due to a dissociation between uniform unimodal stimulation and the natural heterogenous signals in central nervous system). Time-invariant stimulation (e.g., applying stimulation at one frequency) may lead to desensitization of sensations experienced by the patient whereby the lack of novel sensations (e.g., biomimetic sensations) may gradually lose analgesic efficacy.

In various clinical applications of nerve stimulation, unimodal stimulation (e.g., unimodal motor stimulation, unimodal sensory stimulation, or other unimodal stimulation such as but not limited to high frequency, low frequency, sub-threshold, or supra-threshold) relies solely, mostly, or largely on efferent stimulation to elicit muscle contractions that indirectly activate proprioceptive afferent pathways. Applying unimodal stimulation (e.g., motor stimulation, or applying stimulation at one frequency) may lack the complexity (e.g., biomimetic physiological characteristics) needed for engaging or maximizing analgesic mechanisms (e.g., due to translating stimulation through muscle as a filter). Applying unimodal stimulation (e.g., motor stimulation, or applying stimulation at one frequency) may be designed to avoid muscle fatigue by using lower frequencies of stimulation or duty cycles less than 100% and lack robustness or volume of input into the central nervous system that is elicited by directly activating large numbers of sensory fibers. Applying unimodal stimulation (e.g., motor stimulation, or applying stimulation at one frequency) lacks differentiation from normal physiological inputs to the central nervous system such that therapeutic mechanisms are not maximized. Time-invariant stimulation (e.g., applying motor stimulation at one frequency or one duty cycle) may lead to desensitization of sensations experienced by the patient whereby the lack of novel sensations (e.g., biomimetic sensations) may gradually lose analgesic efficacy.

In various clinical applications of nerve stimulation, stimulation of a single nerve, or stimulation of a single sub-population of neural fibers within single nerve (e.g., sensory fibers alone or motor fibers alone), can lead to incomplete coverage of region of pain. A region of pain may be innervated by sensory fibers, motor fibers associated with local muscles, or both. The receptive field of a nerve trunk (e.g., the anatomical locations associated with the sensory and/or motor fibers within the nerve trunk) may overlap with some, most, or all of the patient's region of pain. Applying unimodal stimulation (e.g., motor stimulation alone or sensory stimulation alone, or applying stimulation at one frequency) to a single nerve may not produce robust sensations in the region of pain necessary to produce relief, or may lead to incomplete relief, minimal relief, no relief, and/or waning of pain relief over time (e.g., shorter lasting relief due to a dissociation between uniform unimodal stimulation and the natural heterogenous signals in central nervous system).

In various clinical applications of nerve stimulation, the effectiveness of unimodal stimulation (e.g., unimodal motor stimulation, unimodal sensory stimulation, or other unimodal stimulation such as but not limited to high frequency, low frequency, sub-threshold, or supra-threshold) for the treatment of pain is dependent or partially dependent on targeting a healthy nerve (e.g., a nerve without damage or with minimal damage). For example, unimodal stimulation (e.g., unimodal motor stimulation, unimodal sensory stimulation, or other unimodal stimulation such as but not limited to high frequency, low frequency, sub-threshold, or supra-threshold) may elicit no relief, and/or reduced effectiveness, and/or limited relief, and/or shorter lasting relief in patients with unhealthy nerves and/or nerve damage or pre-existing nerve damage (natural or iatrogenic and/or caused by trauma, chemicals, surgery, diseases, and/or disease progression) such as diabetes (type I and/or II), neuropathy, chemotherapy induced neuropathy, obesity, hypertension, and/or other diseases or disorders.

Therefore, there is a need for improved systems and methods for providing electrical stimulation to treat pain and/or to provide other therapeutic benefits. There is a need for improved systems and methods that are able to provide coordinated stimulating modes (e.g., frequencies) at two or more electrodes while minimizing or preventing certain effects, such as inconsistent and potentially problematic electrochemistry changes at the electrodes and inconsistent and potentially problematic electrochemistry changes at the anode or return electrode.

The various aspects of the disclosed system and method will be described in connection with the placement of one or more leads 12 having one or more electrodes 14, or two or more leadless electrodes, in muscle, adipose, or other tissue, and in electrical proximity to but not contacting nerves, for improved recruitment of targeted nerves for therapeutic purposes, such as for the treatment of pain. That is because the features and advantages that arise due to the disclosed system and method are well suited to this purpose. It is to be appreciated that regions of pain can include any or all portions of the body, whether or not such associated anatomy is physically present on the body at the time of stimulation according to the present system and method, including arms, legs, and trunk in both humans and animals. A portion may not be physically present on the body due to amputation, resection, otherwise removed (e.g., trauma), or it may be congenitally missing.

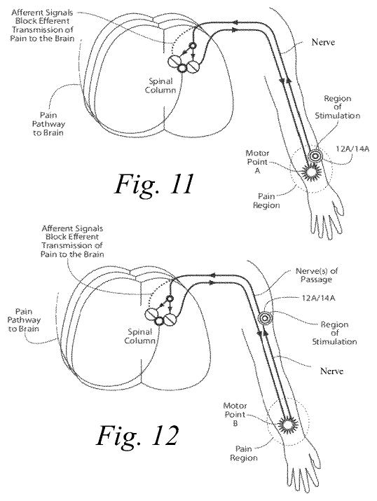

A. Stimulation of Nerves, Their Branches, and Their Innervated Muscles FIG. 11 shows a system and method for stimulating a nerve or muscle A by placing a lead 12(A) with its electrode 14(A) close to motor point A (i.e., stimulating the nerve near the motor point; “motor point” approach). The motor point A is the location where the innervating nerve enters the muscle. At that location, the electrical stimulation intensity required to elicit a full contraction is at the minimum. Any other electrode placement location in the muscle located further from the motor point would require more stimulation intensity to elicit the same muscle contraction.

FIG. 12 shows a system and method for stimulating nerves or their branches, that is unlike the “motor point” system and method shown in FIG. 11, and which incorporates the features of the system and method. As shown in FIG. 12, the system and method identifies a region where there is a local manifestation of pain. The region of pain can comprise, e.g., skin, bone, a joint, connective tissue, muscle, or other tissue or structure. The system and method identify one or more peripheral nerves that are located anatomically upstream or cranial to the region where pain is manifested, through which neural impulses comprising the action potentials that will be interpreted as pain. A given nerve that is identified can comprise a nerve trunk located in a nerve plexus, or a divisions and/or a cord of a nerve trunk, or a nerve branch, provided that it is upstream or cranial of where the nerve innervates the region affected by the pain. The given nerve can be identified by medical professionals using textbooks of human anatomy along with their knowledge of the site and the nature of the pain or injury, as well as by physical manipulated and/or imaging, e.g., by ultrasound, fluoroscopy, or X-ray examination, of the region where pain is manifested. A desired criterion of the selection includes identifying the location of tissue (e.g., muscle, adipose, connective, or other tissue) in electrical proximity to but spaced away from the nerve or nerve branch (e.g., 0.1 cm, 0.1-0.5 cm, 0.5 cm, 0.5-1.0 cm, 1.0 cm, 1-2 cm, or 2 cm), which can be accessed by placement of one or more leads, each with one or more electrodes, aided if necessary by ultrasonic or electro-location techniques. The nerve identified comprises a targeted “nerve of passage.” The muscle identified comprises the “targeted tissue” or “targeted muscle.” In a preferred embodiment, the electrodes are percutaneously inserted using percutaneous leads. In another embodiment, leads are surgically placed and connected to an implanted pulse generator, which may be powered by an implanted battery or wireless power transfer.

The system and method place one or more leads 12(B) with its one more more electrodes 14(B), or two or more leadless electrodes, in the targeted tissue in electrical proximity to (e.g., 0.1-0.5 cm, 0.5-1.0 cm, 1-2 cm, 0.1-3.0 cm), but spaced away from (i.e., not in contact with), the targeted nerve(s) or nerve branch(es). The system and method apply electrical stimulation through the two or more stimulation electrodes to activate electrically or recruit the targeted nerve(s) or nerve branch(es) that convey(s) the neural impulses comprising the pain signal to the spinal column. Alternatively, the system and method may be adapted to enable one or more electrode(s) and/or lead(s) to be in contact with, adjacent to, near, and/or far or remote from the nerve and achieve desirable results.

The system and method can apply electrical stimulation to nerve networks throughout the body. For example, the nerve networks can comprise one or more nerves in the brachia plexus, to treat pain in the chest, shoulders, arms and hands; and/or one or more nerves in the lumbar plexus, to treat pain in the back, abdomen, thighs, knees, and calves; and/or one or more nerves in the sacral plexus, to treat pain in the buttocks, thighs, calves, and feet; and/or one or more nerves in the cervical plexus, to treat pain in the head, neck and shoulders; and/or one or more nerves in the solar plexus, to treat pain or dysfunction in internal organs.

The present system and method provides multimodal (e.g., bimodal) stimulation that coordinates two or more stimulation modalities to overcome the limitations of previous systems and methods of stimulation that were limited to a single stimulation modality (or unimodal) and were unable to provide stimulation that targeted multiple regions of pain via multiple nerves with approaches and therapeutic objectives specific to and best suited for the multiple regions of pain.

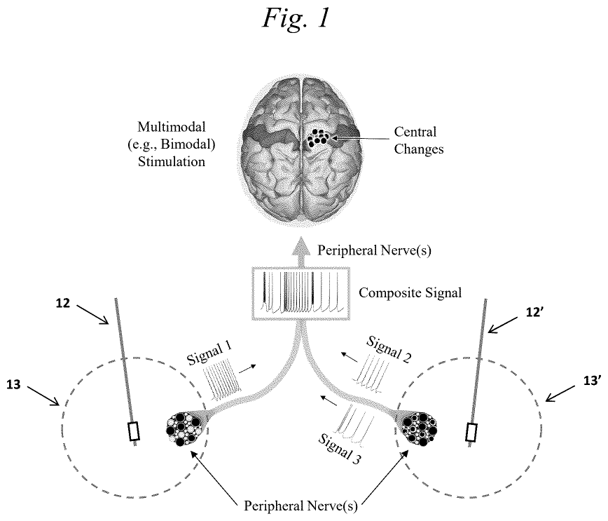

For example, FIG. 1 shows a first lead 12 providing a signal (e.g., stimulation at a frequency) to one peripheral nerve or nerve branch and a second lead 12′ providing the same or a different signal or multiple signals (e.g., stimulation at the same or a different frequency) to another peripheral nerve or nerve branch. In an embodiment, the first lead 12 may comprise one or more electrodes providing a signal (e.g., stimulation at a frequency) to one peripheral nerve or nerve branch and the second lead 12 may comprise one or more electrodes providing the same or a different signal or multiple signals (e.g., stimulation at the same or a different frequency) to another peripheral nerve or nerve branch. Together the system may provide a coordinated composite neural signal which travels from the periphery along the peripheral nerve toward the central nervous system to elicit central changes in the brain and provide pain relief to systems or regions disrupted by disease, trauma, overuse, or otherwise subject to maladaptive changes. The dotted circles around the electrodes represent stimulation waveforms or the area or zone of activation 13, 13′ corresponding to the first and second leads 12, 12′ (e.g., area that is being activated by stimulation). Neural signals directly or indirectly activated by stimulation are shown in FIG. 1, including the complex, composite neural signal.

As a non-limiting example, while some nerve targets are conducive to or best suited for stimulation that activates sensory fibers in the target nerve to produce pain relief, other nerve targets are conducive to or best suited for stimulation that activates motor fibers or a mix of sensory and motor fibers in the target nerve to produce pain relief. It may be undesirable to activate motor fibers or a mix of motor fibers and sensory fibers in some stimulation applications, whereas it may be undesirable to activate sensory fibers only in other stimulation applications, even if the combination of these two applications would increase, optimize, maximize, or produce pain relief that is superior, greater, preferred, or better than the pain relief produced by either stimulation application individually. The present system and method can overcome these limitations of previous approaches by delivering and/or coordinating two or more stimulation modalities or application of bimodal or multimodal stimulation to any combination of peripheral nerves or nerve branches throughout the body, for example producing pain relief through the activation of different types or populations of fibers or activating the same populations or types of fibers with different stimulation parameters to increase, optimize, maximize, or produce pain relief that is superior, greater, preferred, or better than the pain relief produced by either stimulation application individually. Non-limiting examples of combinations of nerve targets and pain regions include: the suprascapular nerve and axillary nerve to treat pain in the shoulder; the lumbar spinal nerve and the lumbar medial branch of the dorsal ramus nerve to treat pain in the back and legs; the femoral nerve and the distal branch of the femoral nerve at the vastus medialis obliquus to treat pain in the knee; the occipital nerve and the cervical medial branch of the dorsal ramus nerve to treat pain in the head and neck; the brachial plexus and the cervical medial branch of the dorsal ramus nerve to treat pain in the neck and arms. This approach may also be applied to multiple locations along the same nerve and/or any combination of nerves and their distal branches.

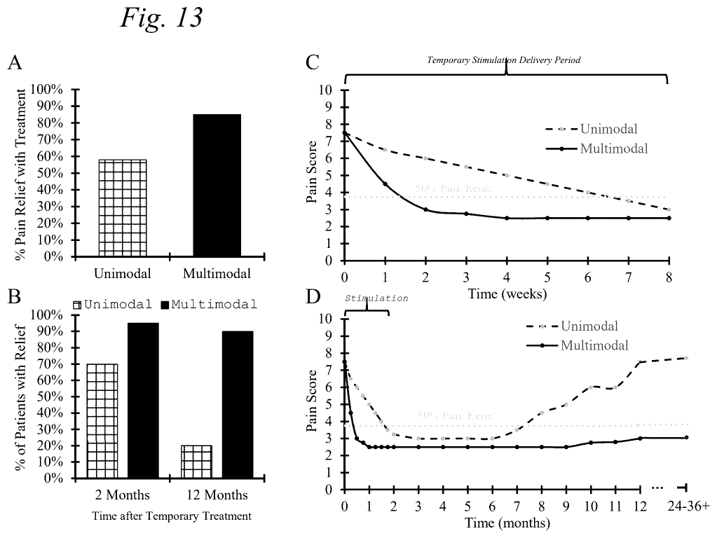

In an example, FIG. 13A-D illustrates exemplary outcomes after a temporary treatment from systems providing unimodal stimulation (unimodal: sensory or unimodal: motor) versus multimodal (e.g., bimodal) stimulation which simultaneously, concurrently, interleaved, and/or in another pattern provides signals to mimic the natural coordination between sensory and motor signals in the central nervous system to provide pain relief to systems or regions disrupted by disease, trauma, overuse, or otherwise subject to maladaptive changes.

FIG. 13A illustrates exemplary outcomes in percent pain relief with treatment, where multimodal (e.g., bimodal) stimulation results in increased average pain relief (e.g., better pain relief, higher percent relief, larger average reduction in pain score) compared to unimodal stimulation. FIG. 13B illustrates exemplary outcomes in responder rate (proportion of patients with greater than or equal to 50% pain relief), where multimodal (e.g., bimodal) stimulation results in an increased proportion of patients who experience relief (e.g., increased likelihood of relief, increased likelihood for successful treatment) compared to unimodal stimulation at 2 months and 12 months after treatment. FIG. 13C illustrates exemplary outcomes in the amount of time to experience relief, where multimodal (e.g., bimodal) stimulation results in faster relief (e.g., avoiding a delayed response, decreasing time to response, increasing number of early responders, reducing number of delayed responders, overcoming issues with compliance that delay response) compared to unimodal stimulation. FIG. 13D illustrates exemplary outcomes in pain scores over time (e.g., 0-12 months, 12-36 months, greater than 36 months), where multimodal (e.g., bimodal) stimulation results in more durable (longer lasting, more resilient, relief that is less likely to fade) relief compared to unimodal stimulation.

As shown in FIG. 13A, a temporary treatment with multimodal (e.g., bimodal) stimulation may result in a greater reduction in pain relief than with a temporary treatment with unimodal stimulation. As shown in FIG. 13B, a temporary treatment with multimodal (e.g., bimodal) stimulation may result in a greater proportion of patients with greater than or equal to 50% relief in the short term (e.g., 2 months) and long term (e.g., 12 months) than with a temporary treatment with unimodal stimulation. As shown in FIG. 13C, a temporary treatment with multimodal (e.g., bimodal) stimulation may result in more rapid relief (e.g., quicker relief, less time until 50% pain relief) than with a temporary treatment with unimodal stimulation. As shown in FIG. 13D, a temporary treatment with multimodal (e.g., bimodal) stimulation may result in more durable pain relief (e.g., longer lasting relief) than with a temporary treatment with unimodal stimulation.

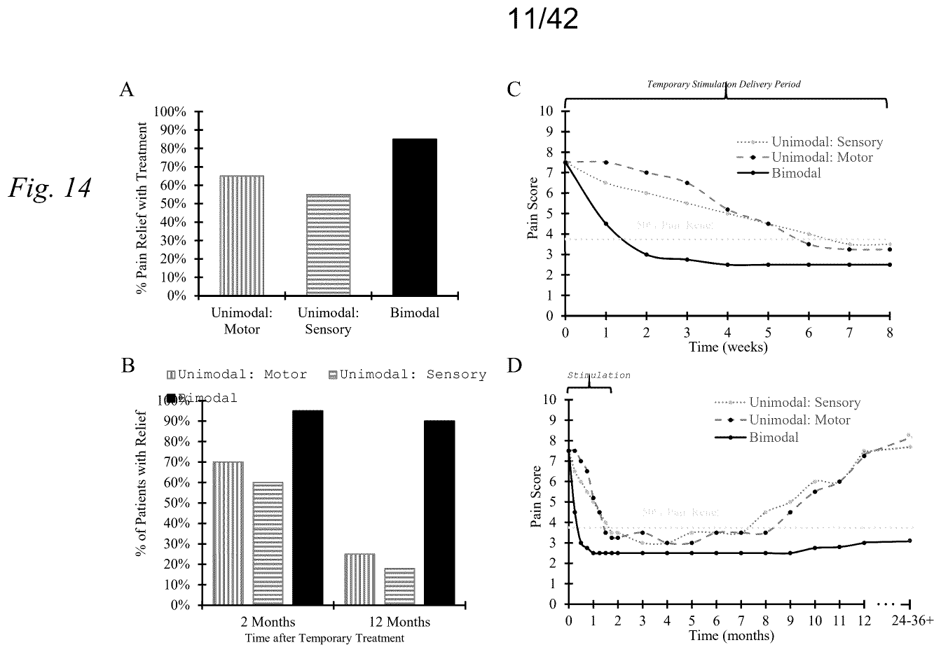

In an example, FIG. 14A-D illustrates exemplary outcomes after a temporary treatment from systems providing unimodal motor stimulation or unimodal sensory stimulation versus multimodal (e.g., bimodal) stimulation.

FIG. 14A illustrates exemplary outcomes in percent pain relief with treatment, where multimodal (e.g., bimodal) stimulation results in increased average pain relief (e.g., better pain relief, higher percent relief, larger average reduction in pain score) compared to unimodal motor stimulation and unimodal sensory stimulation. FIG. 14B illustrates exemplary outcomes in responder rate (proportion of patients with greater than or equal to 50% pain relief), where multimodal (e.g., bimodal) stimulation results in an increased proportion of patients who experience relief (e.g., increased likelihood of relief, increased likelihood for successful treatment) compared to unimodal motor stimulation and unimodal sensory stimulation at 2 months and 12 months after treatment. FIG. 14C illustrates exemplary outcomes in the amount of time to experience relief, where multimodal (e.g., bimodal) stimulation results in faster (e.g., avoiding a delayed response, decreasing time to response, increasing number of early responders, reducing number of delayed responders, overcoming issues with compliance that delay response) compared to unimodal motor stimulation and unimodal sensory stimulation. FIG. 14D illustrates exemplary outcomes in pain scores over time (e.g., 0-12 months, 12-36 months, greater than 36 months), where multimodal (e.g., bimodal) stimulation results in more durable (longer lasting, more resilient, relief that is less likely to fade) relief compared to unimodal motor stimulation and unimodal sensory stimulation.

As shown in FIG. 14A, a temporary treatment with multimodal (e.g., bimodal) stimulation may result in greater pain relief than with a temporary treatment with unimodal motor stimulation or a temporary treatment with unimodal sensory stimulation. As shown in FIG. 14B, a temporary treatment with multimodal (e.g., bimodal) stimulation may result in a greater proportion of patients with greater than or equal to 50% relief in the short term (e.g., 2 months) and long term (e.g., 12 months) than with a temporary treatment with unimodal stimulation motor stimulation or unimodal sensory stimulation. As shown in FIG. 14C, a temporary treatment with multimodal (e.g., bimodal) stimulation may result in more rapid relief (e.g., quicker relief, less time until 50% pain relief) than with a temporary treatment with unimodal motor stimulation or unimodal sensory stimulation. As shown in FIG. 14D, a temporary treatment with multimodal (e.g., bimodal) stimulation may result in more durable pain relief (e.g., longer lasting relief) than with a temporary treatment with unimodal motor stimulation or unimodal sensory stimulation.

It is appreciated that the concepts illustrated in FIG. 14 may be similarly applied to other forms of unimodal stimulation (Unimodal X and Unimodal Y), for example, but not limited to, sensory stimulation, motor stimulation, stimulation with low frequencies (e.g., 1-20, 2-18, 4-16, 6-16, 8-14, 8-16, 10-16, 10-14, 10-12, 12, 4-20 Hz), stimulation with higher frequencies (e.g., 20-35, 35-45, 45-60, 60-70, 70-85, 85-120, 20-120, 60-120, 96, 120, 120-200, 120-500, 200-500, 500-1500, 500-5000, 5000-15000, 20-15000 Hz), sub-perception stimulation, sub-threshold stimulation, super-threshold stimulation, threshold stimulation, burst stimulation, other stimulation patterns, or other waveforms.

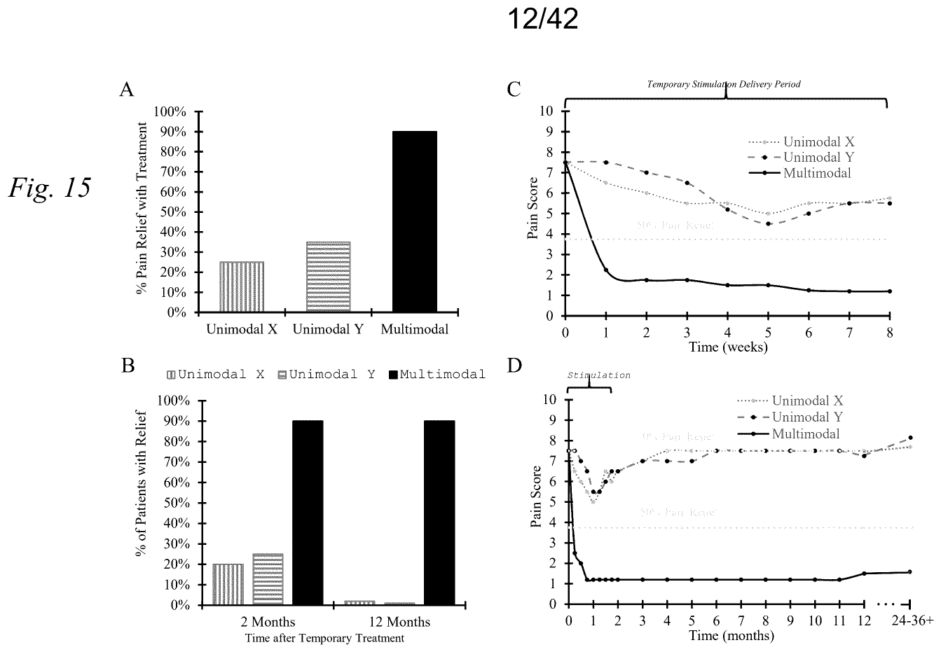

FIG. 15A-D illustrates exemplary outcomes after a temporary treatment from systems providing unimodal X stimulation or unimodal X stimulation versus multimodal (e.g., bimodal) stimulation. As illustrated in FIG. 15A-D, treatment with multimodal stimulation may result in a greater percentage of pain relief (FIG. 15A), may provide relief in a greater proportion of patients (FIG. 15B), may take less time to develop relief (FIG. 15C), and may provide more durable (longer lasting) relief (FIG. 15D) than in Unimodal X and Unimodal Y stimulation.

It is appreciated that unimodal X or unimodal Y stimulation may correspond to sensory stimulation, motor stimulation, stimulation at lower frequencies (e.g., 4-20 Hz), stimulation at higher frequencies (e.g., 20-60 Hz, 60-120 Hz, 120-500 Hz, 500-15000 Hz, 1000-20000 Hz), sub-threshold stimulation, burst stimulation, or other stimulation patterns or waveforms. FIG. 15A illustrates exemplary outcomes in percent pain relief with treatment, where multimodal (e.g., bimodal) stimulation results in increased average pain relief (e.g., better pain relief, higher percent relief) compared to unimodal X and Y stimulation. FIG. 15B illustrates exemplary outcomes in responder rate (proportion of people with greater than or equal to 50% pain relief), where multimodal (e.g., bimodal) stimulation results in an increased proportion of patients who experience relief (e.g., increased likelihood of relief) compared to unimodal X and Y stimulation at 2 months and 12 months after treatment. FIG. 15C illustrates exemplary outcomes in the amount of time to experience relief, where multimodal (e.g., bimodal) stimulation results in faster relief (e.g., avoiding a delayed response, decreasing time to response, increasing number of early responders, avoiding issues with compliance) compared to unimodal X and Y stimulation. FIG. 15D illustrates exemplary outcomes in pain scores over time (e.g., 0-12 months, 12-36 months, greater than 36 months), where multimodal (e.g., bimodal) stimulation results in more durable (longer lasting) relief compared to unimodal X and Y stimulation.

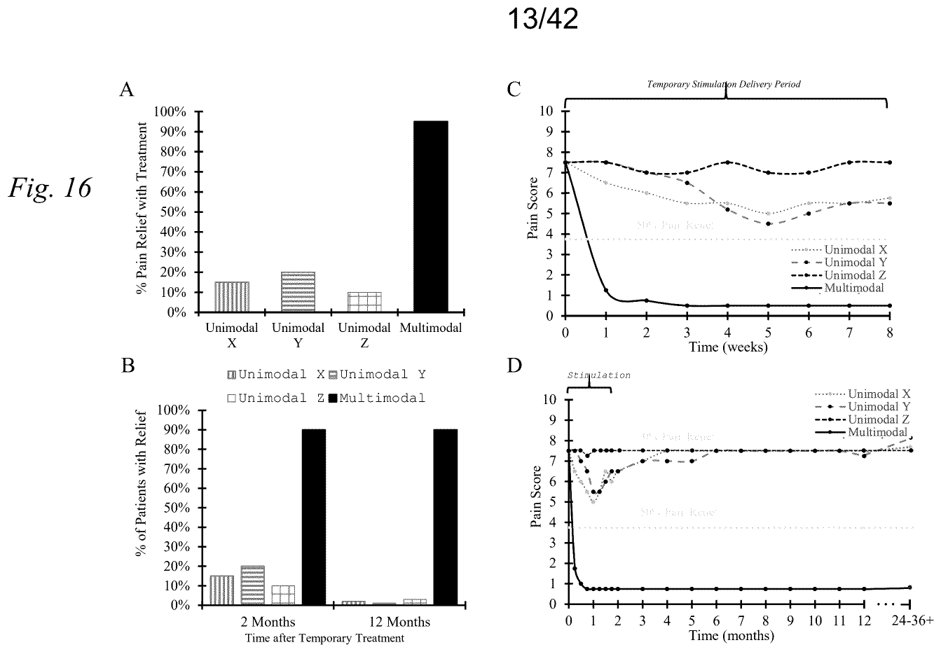

FIG. 16A-D illustrates exemplary outcomes after a temporary stimulation delivery period with multimodal (e.g., bimodal) stimulation in patients with unhealthy nerves and/or nerve damage or pre-existing nerve damage (natural or iatrogenic and/or caused by trauma, chemicals, surgery, diseases, and/or disease progression) such as, but not limited to, diabetes (type I and/or II), neuropathy, chemotherapy induced neuropathy, obesity, hypertension, and/or other diseases or disorders.

FIG. 16A-D reveals how, in patients with unhealthy nerves and/or nerve damage or pre-existing nerve damage (natural or iatrogenic and/or caused by trauma, chemicals, surgery, diseases, and/or disease progression) such as, but not limited to, diabetes (type I and/or II), neuropathy, chemotherapy induced neuropathy, obesity, hypertension, and/or other diseases or disorders, Unimodal Z Stimulation (e.g., sensory stimulation, motor stimulation, stimulation at lower frequencies (e.g., 1-20, 2-18, 4-16, 6-16, 8-14, 8-16, 10-16, 10-14, 10-12, 12, 4-20 Hz), stimulation within, at, approximately at, or over a range of frequencies (e. g,. with a lower bound chosen from one or more of 1, 2, 3, 4, 5, 6, 7, 8, 9, 10, 11, 12, 13, 14, 15, 16, 17, 18, 19, and/or 20, and/or with an upper bound chosen from one or more of 20, 19, 18, 17, 16, 15, 14, 13, 14, 12, 11, 10, 9, 8, 7, 6, 5, 4, 3, 2, and/or 1), stimulation at higher frequencies (e.g., 20-35, 35-45, 45-60, 60-70, 70-85, 85-120, 20-120, 60-120, 96, 120, 120-200, 120-500, 200-500, 500-1500, 500-5000, 5000-15000, 20-15000 Hz), sub-threshold stimulation, burst stimulation, or other stimulation patterns or waveforms) provides little, minimal, reduced, or inadequate pain relief, while multimodal (e.g., bimodal) stimulation may coordinate different modes (e.g., frequencies) in various fashions to produce the synergistic effect, for example, simultaneously, concurrently, interleaved, and/or in another pattern in order to mimic the natural coordination between sensory and motor signals in the central nervous system, provide biomimetic signals (e.g., signals that match, mimic, or resemble natural sensations), or provide signals outside the realm of natural sensations in order to provide pain relief to systems or regions disrupted by disease, trauma, overuse, or otherwise subject to maladaptive changes.

FIG. 16A illustrates that, in patients with unhealthy nerves and/or nerve damage, a system with multimodal (e.g., bimodal) stimulation provides a greater percentage of relief compared to a system with unimodal stimulation X, Y, and Z (e.g., sensory stimulation, motor stimulation, stimulation at lower frequencies (e.g., 4-20 Hz), stimulation at higher frequencies (e.g., 20-60 Hz, 60-120 Hz, 120-500 Hz, 500-15000 Hz, 1000-20000 Hz), sub-threshold stimulation, burst stimulation, or other stimulation patterns or waveforms).

FIG. 16B illustrates that, in patients with unhealthy nerves and/or nerve damage, a system with multimodal (e.g., bimodal) stimulation provides a greater percentage of relief and a quicker relief compared to a system with unimodal stimulation X, Y, and Z (e.g., sensory stimulation, motor stimulation, stimulation at lower frequencies (e.g., 4-20 Hz), stimulation at higher frequencies (e.g., 20-60 Hz, 60-120 Hz, 120-500 Hz, 500-15000 Hz, 1000-20000 Hz), sub-threshold stimulation, burst stimulation, or other stimulation patterns or waveforms).

FIG. 16C illustrates that a system with multimodal (e.g., bimodal) stimulation provides relief to a greater proportion of patients with unhealthy nerves and/or nerve damage compared to unimodal stimulation X, Y, and Z (e.g., sensory stimulation, motor stimulation, stimulation at lower frequencies (e.g., 4-20 Hz), stimulation at higher frequencies (e.g., 20-60 Hz, 60-120 Hz, 120-500 Hz, 500-15000 Hz, 1000-20000 Hz), sub-threshold stimulation, burst stimulation, or other stimulation patterns or waveforms).

FIG. 16D illustrates that, in patients with unhealthy nerves and/or nerve damage, a system with multimodal (e.g., bimodal) stimulation provides a longer lasting relief compared to a system with unimodal stimulation X, Y, and Z (e.g., sensory stimulation, motor stimulation, stimulation at lower frequencies (e.g., 4-20 Hz), stimulation at higher frequencies (e.g., 20-60 Hz, 60-120 Hz, 120-500 Hz, 500-15000 Hz, 1000-20000 Hz), sub-threshold stimulation, burst stimulation, or other stimulation patterns or waveforms). This illustrative example demonstrates how multimodal stimulation may provide a synergistic effect on outcomes by generating results that are greater than the simple addition of effects from multiple unimodal stimulation paradigms.

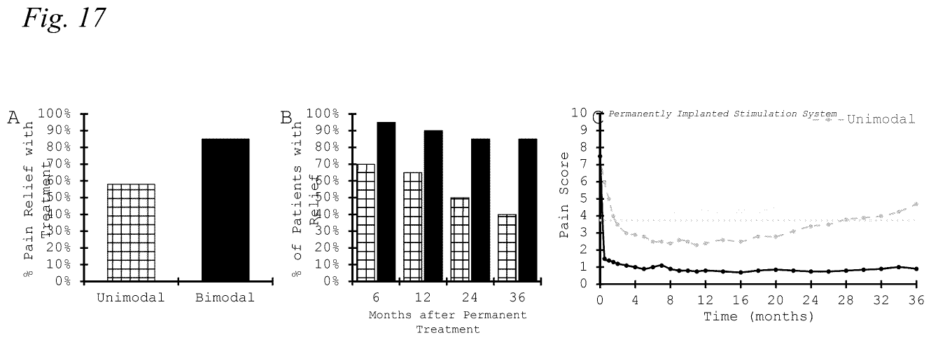

FIG. 17A-C illustrates exemplary outcomes after a permanently implanted stimulation system provides unimodal motor stimulation or unimodal sensory stimulation versus multimodal (e.g., bimodal) stimulation.

As shown in FIG. 17A, a permanently implanted system with multimodal (e.g., bimodal) stimulation (e.g., in which more than one mode may be provided simultaneously, concurrently, interleaved, and/or in another pattern) may result in a greater reduction in pain relief and/or improvement in function, symptoms, and/or outcomes than with a permanently implanted system with unimodal stimulation or with more than one mode that can only be provided one at a time or must be selected or chosen by the user but cannot be provided simultaneously to one nerve and/or multiple nerves or nerve targets. FIG. 17A illustrates exemplary outcomes in percent pain relief with treatment, where multimodal (e.g., bimodal) stimulation results in increased average pain relief (e.g., better pain relief, higher percent relief) compared to unimodal stimulation.

As shown in FIG. 17B, a permanently implanted system with multimodal (e.g., bimodal) stimulation may result in a greater proportion of patients with greater than or equal to a target or desired level of pain relief (e. g, 50% relief) over time (e.g., 6 months, 12 months, 24 months, and 36 months) than with a permanently implanted system with unimodal stimulation. FIG. 17B illustrates exemplary outcomes in responder rate (proportion of patients with greater than or equal to 50% pain relief), where multimodal (e.g., bimodal) stimulation results in an increased proportion of patients who experience relief (e.g., increased likelihood of relief, increased likelihood for successful treatment) compared to unimodal stimulation at 6 months, 12 months, 24 months, and 36 months after treatment.

As shown in FIG. 17C, a permanently implanted system with multimodal (e.g., bimodal) stimulation may result in quicker (e.g., less time until the target or desired level of pain relief, such as 50% relief, is achieved) and more durable pain relief (e.g., long lasting relief) than with a permanently implanted system with unimodal stimulation. FIG. 17C illustrates exemplary outcomes in pain scores over time (e.g., 0-36 months, greater than 36 months), where multimodal (e.g., bimodal) stimulation results in more durable (longer lasting, more resilient, relief that is less likely to fade) relief compared to unimodal stimulation.

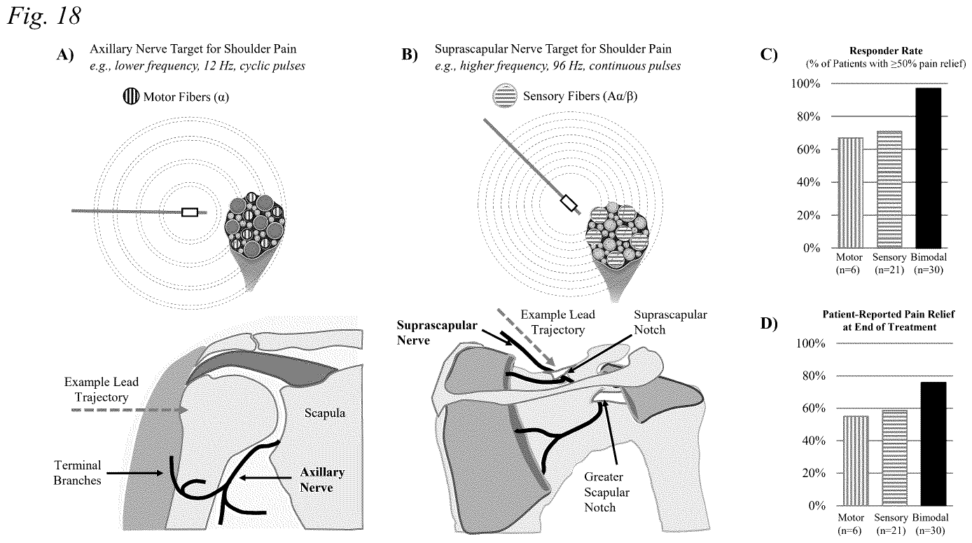

FIG. 18A-D is a schematic view of the shoulder anatomy showing example nerve targets for a system to apply (A) motor stimulation (e.g., cyclic pulses at 12 Hz) to the axillary nerve at the deltoid and (B) sensory stimulation (e.g., continuous pulses at 100 Hz) to the suprascapular nerve and/or axillary nerves for the treatment of shoulder pain (i.e., single or dual lead unimodal sensory stimulation), where the dotted circles around the electrodes represent stimulation waveforms or the area or zone of activation (e.g., area that is being activated).

For example, as shown in FIG. 18A-D, if the region of pain is in, primarily in, partially in, overlaps with, and/or encompasses the shoulder, the system and method can identify and stimulate the suprascapular nerve and/or the axillary nerve to treat the region of pain in the shoulder. The system may apply electrical stimulation to the suprascapular nerve and/or the axillary nerve concurrently (e.g., stimulation pulses are delivered from the one or more electrode to each target nerve in concurrent, overlapping, simultaneous, interleaved, and/or coinciding periods of time or durations of therapy) or at different times (e.g., stimulation pulses are delivered from the one or more electrode to each target nerve in non-overlapping, alternating, rotating, consecutive, overlapping, and/or patterned sequences of periods of time or durations of therapy on the scale of milliseconds, seconds, minutes, hours, days, months, and/or years).

In FIG. 18C and FIG. 18D, patient-reported pain relief at end-of-treatment were collected from a single medical center among patients who underwent peripheral nerve stimulation treatment for shoulder pain (n=57). (C) On average, patients with a system that coordinates both motor stimulation and sensory stimulation (e.g., a “Bimodal” stimulation system) achieve a higher responder rate (proportion of patients with greater than or equal to 50% pain relief after a 60-day treatment compared to start of treatment) than patients with a system that delivers motor stimulation only or sensory stimulation only. (D) On average, patients with a system that coordinates both motor stimulation and sensory stimulation (e.g., a “Bimodal” stimulation system) report a greater percent of pain relief at the end of a 60-day treatment than patients with a system that delivers motor stimulation only or sensory stimulation only.

As a non-limiting example, in one embodiment, the at least one electrodes may deliver stimulation to the target nerves in alternating periods of 6 hours such that the first nerve receives 6 hours of stimulation then the second nerve receives 6 hours of stimulation with no overlap or period of concurrent stimulation. As another non-limiting example, the at least one electrodes may deliver stimulation to the target nerves in overlapping or partially overlapping periods of time, for example the first nerve receives 6 hours of stimulation and the second nerve receives 6 hours of stimulation at the same time, or the first nerve receives 6 hours of stimulation per day and the second nerve receives continuous (e.g., 24 hours per day) stimulation (e.g., stimulation periods overlap for 6 hours per day), or the first nerve receives 6-12 hours of stimulation per day and the second nerve receives continuous (e.g., 24 hours per day) stimulation (e.g., stimulation periods overlap for 6-12 hours per day). The system may apply electrical stimulation to the suprascapular nerve and/or the axillary nerve at the same frequency or at different frequencies. If electrical stimulation activates one or more target nerves sufficiently, then the patient may feel a comfortable tingling sensation and/or muscle contraction or tension, which overlaps with the region of pain and/or otherwise reduces pain.

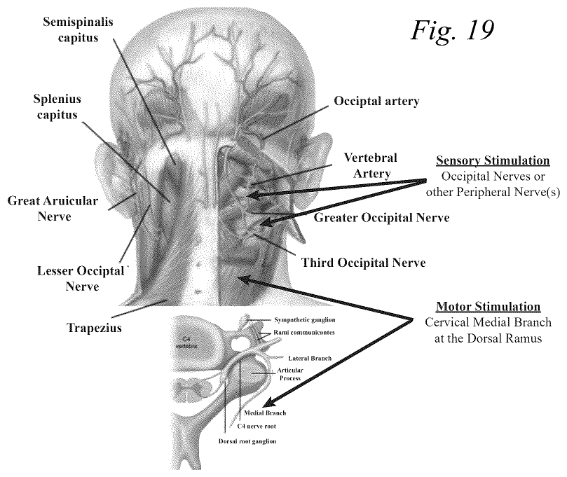

For example, as shown in FIG. 19, if the region of pain is in, primarily in, partially in, overlaps with, and/or encompasses the head and neck, the system and method can identify and stimulate the occipital nerve and the cervical medial branch of the dorsal ramus nerve to treat pain in the head and neck. The system may apply electrical stimulation to the occipital nerve and/or the cervical medial branch of the dorsal ramus nerve concurrently (e.g., stimulation pulses are delivered from the one or more electrode to each target nerve in concurrent, overlapping, simultaneous, and/or coinciding periods of time or durations of therapy) or at different times (e.g., stimulation pulses are delivered from the one or more electrode to each target nerve in non-overlapping, alternating, rotating, consecutive, or patterned sequences of periods of time or durations of therapy on the scale of milliseconds, seconds, minutes, hours, and/or days). The system may deliver electrical stimulation to the occipital nerve and/or the cervical medial branch of the dorsal ramus nerve at the same frequency or at different frequencies. If electrical stimulation activates one or more target nerves sufficiently at the correct intensity, then the patient may feel comfortable sensations and/or muscle contractions that overlap with the region of pain and/or otherwise reduce the pain.

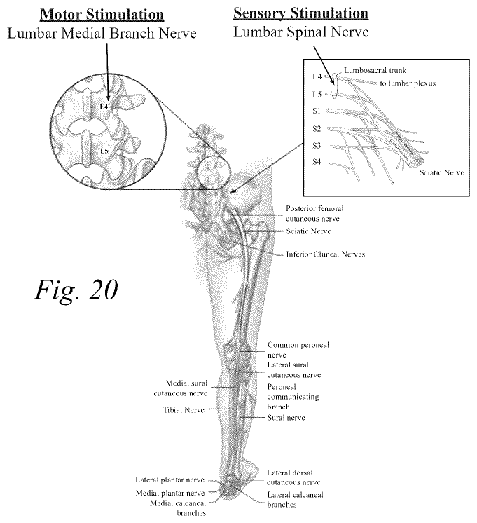

For example, as shown in FIG. 20, if the region of pain is in, primarily in, partially in, overlaps with, and/or encompasses the low back and legs, the system and method can identify and stimulate the lumbar spinal nerve and the lumbar medial branch of the dorsal ramus nerve to treat pain in the back and legs. The system may deliver electrical stimulation to the lumbar spinal nerve and/or the lumbar medial branch of the dorsal ramus nerve at the same time or at different times. The system may apply electrical stimulation to the lumbar spinal nerve and/or the lumbar medial branch of the dorsal ramus nerve at the same frequency or at different frequencies. If electrical stimulation activates one or more target nerves sufficiently at the correct intensity, then the patient may feel comfortable sensations and/or muscle contractions that overlap with the region of pain and/or otherwise reduces pain.

It is to be appreciated that the sensations generated by electrical stimulation could be described with other words such as buzzing, thumping, tapping, tingling, pulsing, vibrating, warm, etc. Evoking sensations, such as paresthesias or contractions and/or tension of the muscle(s) in the region of pain confirms correct lead placement and indicates stimulus intensity is sufficient to reduce pain. Inserting a lead 12 percutaneously allows the lead 12 to be placed quickly and easily, and placing the lead 12 in a peripheral location, e.g., muscle, where it is less likely to be dislodged, addresses the lead migration problems of spinal cord stimulation that result in decreased paresthesia coverage, decreased pain relief, and the need for frequent patient visits for reprogramming.

Placing the lead 12 percutaneously in muscle in electrical proximity to but spaced away from the targeted nerve or nerve branch minimizes complications related to lead placement and movement. In a percutaneous system, an electrode lead 12, such as a coiled fine wire electrode lead may be used because it is minimally-invasive and well suited for placement in proximity to a nerve or nerve branch. The lead can be sized and configured to withstand mechanical forces and resist migration during long-term use, particularly in flexible regions of the body, such as the shoulder, elbow, and knee.

### B. the Lead

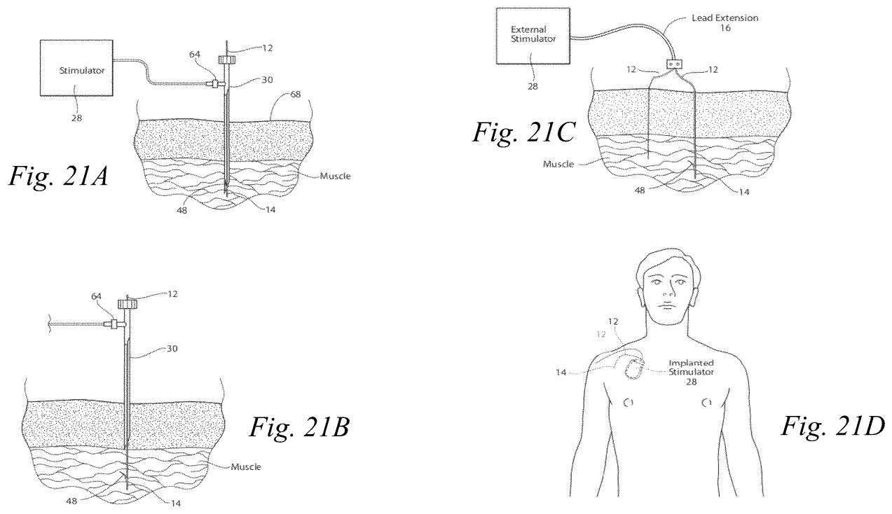

As FIG. 21A shows, the lead can comprise of one or more electrodes, e.g., a fine wire electrode 14, paddle electrode, intramuscular electrode, or general-purpose electrode, inserted via a needle introducer 30 or surgically implanted in proximity of a targeted nerve or nerve branch. Once proper placement is achieved, the needle introducer 30 may be withdrawn (as FIGS. 21B and 12C show), leaving the two or more electrodes attached to one or more leads, or two or more leadless electrodes in place. Stimulation may also be applied through one or more penetrating electrodes, such as an electrode array comprised of any number (i.e., one or more) of needle-like electrodes that are inserted into the target site. In both cases, the lead(s) may placed using a needle-like introducer 30, allowing the placement of one or more leads and one or more electrodes to be minimally invasive, though surgical placement could also be utilized.

In a representative embodiment, the lead 12 comprises a thin, flexible component made of a metal and/or polymer material. By “thin,” it is preferred that the lead may be approximately 0.75 mm (0.030 inch) or less in diameter.

The lead 12 may comprise one or more conductors, e.g., one or more coiled metal wires, disposed within an open or flexible elastomer core. The wire can be insulated, e.g., with a biocompatible polymer film, such as polyfluorocarbon, polyimide, or parylene. The lead is desirably coated with a textured, bacteriostatic material, which helps to stabilize the lead in a way that still permits easy removal at a later date and increases tolerance.

The lead(s) 12 may be electrically insulated everywhere except at one (monopolar), or two (bipolar), or three (tripolar) or more, for example, conduction locations, or electrodes near its distal tip. Each of the conduction locations may be connected to one or more conductors that run the length of the lead and lead extension 16 (see FIG. 21C), proving electrical continuity from the conduction location through the lead 12 to an external pulse generator or stimulator 28 (see FIG. 21C) or an implanted pulse generator or stimulator 28 (see FIG. 21D).

The conduction location(s) or electrode(s) 14 may comprise a de-insulated area of an otherwise insulated conductor that runs the length of an entirely insulated electrode. The de-insulated conduction region of the conductor can be formed differently, e.g., it can be wound with a different pitch, or wound with a larger or smaller diameter, or molded to a different dimension. The conduction location(s) or the electrode(s) 14 may comprise a separate material (e.g., metal or a conductive polymer) exposed to the body tissue to which the conductor of the wire is electrically coupled.

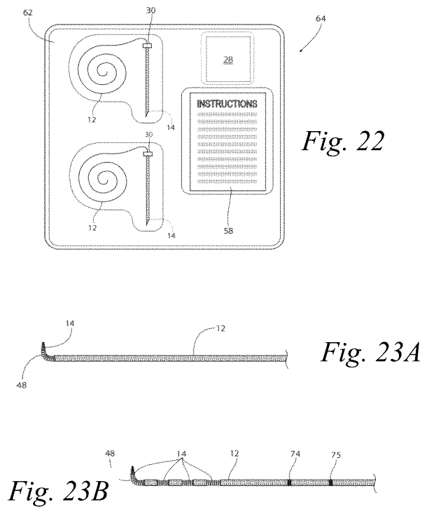

The lead(s) 12 is desirably provided in a sterile package 62 (see FIG. 22), and may be pre-loaded in the introducer needle 30. The package 62 can take various forms and the arrangement and contents of the package 62. As shown in FIG. 22, the package 62 comprises a sterile, wrapped assembly. The package 62 includes an interior tray made, e.g., from die cut cardboard, plastic sheet, or thermo-formed plastic material, which hold the contents. The package 62 also desirably includes instructions for use 58 for using the contents of the package to carry out the lead location and placement procedures, as will be described in greater detail below.

The lead(s) 12 desirably possesses mechanical properties in terms of flexibility and fatigue life that provide an operating life free of mechanical and/or electrical failure, taking into account the dynamics of the surrounding tissue (e.g., stretching, bending, pushing, pulling, crushing, etc.). The material of the electrode(s) 14 desirably discourages the in-growth of connective tissue along its length, so as not to inhibit its withdrawal at the end of its use. However, it may be desirable to encourage the in-growth of connective tissue at the distal tip of the electrode(s) and/or along the length of the lead, to enhance its anchoring in tissue.

One embodiment of the lead 12 shown in FIG. 23A may comprise a minimally invasive coiled fine wire lead 12 and electrode(s) 14. The electrode(s) 14 may also include, at its distal tip, an anchoring element 48. In the illustrated embodiment, the anchoring element 48 takes the form of a simple barb or bend (see also FIG. 21C). The anchoring element 48 is sized and configured so that, when in contact with tissue, it takes purchase in tissue, to resist dislodgement or migration of the electrode out of the correct location in the surrounding tissue. Desirably, the anchoring element 48 is prevented from fully engaging body tissue until after the electrode 14 has been correctly located and deployed.

An alternative embodiment of an electrode lead 12 shown in FIGS. 23A and 23B, may also include, at or near its distal tip or region, one or more anchoring element(s) 70. In the illustrated embodiment in FIG. 24A-B, the anchoring element 70 takes the form of an array of shovel-like paddles or scallops 76 proximal to the proximal-most electrode 14 (although a paddle 76 or paddles could also be proximal to the distal most electrode 14, or could also be distal to the distal most electrode 14) The paddles 76 as shown are sized and configured so they will not cut or score the surrounding tissue. The anchoring element 70 is sized and configured so that, when in contact with tissue, it takes purchase in tissue, to resist dislodgement or migration of the electrode out of the correct location in the surrounding tissue (e.g., muscle 54). Desirably, the anchoring element 70 is prevented from fully engaging body tissue until after the electrode 14 has been deployed. The electrode is not deployed until after it has been correctly located during the implantation (lead placement) process, as previously described. In addition, the lead 12 may include one or more ink markings 74, 75 (shown in FIGS. 23B and 24A) to aid the physician in its proper placement.

Alternatively, or in combination, stimulation may be applied through any type of nerve cuff (spiral, helical, cylindrical, book, flat interface nerve electrode (FINE), slowly closing FINE, etc.), paddle (or paddle-style) electrode lead, cylindrical electrode lead, coiled lead, open coiled lead, open coil lead, closed coil, close coil lead, tubed lead, and/or other lead that is surgically or percutaneously placed, inserted, implanted, temporarily and/or permanently implanted within tissue at the target site.

In all cases, the lead(s) may exit through the skin and connect with one or more external stimulators 28 (shown in FIG. 21C), or the lead(s) may be routed subcutaneously or within the body without exiting the skin to one or more implanted pulse generators 28 (shown in FIG. 21D), or they may be connected as needed to internal and external coils for RF (Radio Frequency) wireless telemetry communications or an inductively coupled telemetry to control the implanted pulse generator, and/or the lead(s) may not need to be connected to a received or stimulator (e.g., if the lead contains a stimulator and/or receiver and/or if it is preconnected with or terminated in or on a stimulator, receiver, pulse generator, conductor, and/or has an apparatus and/or method for receiving or producing power and/or stimulation). As shown in FIG. 21D, the implanted pulse generator 28 may be located some distance (remote) from the electrode(s) 14, or an implanted pulse generator and/or receiver may be integrated with the lead and/or an electrode(s) (not shown), eliminating the need to route the lead subcutaneously to the implanted pulse generator.

The introducer 30 (see FIG. 21A) may be insulated along the length of the shaft, except for those areas that correspond with the exposed conduction surfaces of the electrode 14 housed inside the introducer 30. These surfaces on the outside of the introducer 30 are electrically isolated from each other and from the shaft of the introducer 30. These surfaces may be electrically connected to a connector 64 at the end of the introducer body (see FIG. 21A). This allows connection to an external stimulator 28 (shown in FIG. 21A) during the implantation process. Applying stimulating current through the outside surfaces of the introducer 30 provides a close approximation to the response that the electrode 14 will provide when it is deployed at the current location of the introducer 30.

The introducer 30 may be sized and configured to be bent by hand prior to its insertion through the skin. This will allow the physician to place lead 12 in a location that is not in an unobstructed straight line with the insertion site. The construction and materials of the introducer 30 allow bending without interfering with the deployment of the lead 12 and withdrawal of the introducer 30, leaving the lead 12 in the tissue.

### C. Insertion of the Lead

Representative lead insertion techniques will now be described to place one or more electrode leads 12 in muscle in one or more desired locations that are in electrical proximity to but spaced away from the target nerves or their branches. It is this lead placement that makes possible the use of one or more stimulation modalities to any combination of nerve in the peripheral nervous system throughout the body with a single system using one or more leads 12 to provide pain relief.

Instructions for use 58 (see FIG. 22) can direct use of system and method for the placement of lead 12 in muscle in electrical proximity to but spaced away from the nerve or nerve branch for improved recruitment of target nerves, e.g., with the placement of one or more leads connected to one or more systems 12. The instructions for use may include instructions for placing a lead 12 for the activation of the targeted nerve or branch in a system for the relief of pain, for example. The instructions for use may also include instructions for recording stimulus parameters, including intensity associated with a first sensation of stimulation, a first noticeable muscle contraction, and/or a maximum tolerable contraction at multiple locations, which can be used to aid in determining desired stimulation parameters for optimal stimulation. The instructions for use may also include instructions for using a variety of stimulating frequencies (e.g., 0.5-12 Hz or 0.1-20 Hz, or 0.05-40 Hz, 20-120 Hz, or 12-200 Hz, or 200-500 Hz, or 500-15,000 Hz, or 1,500 Hz, or 5,000 Hz, or 15,000 Hz, or 20,000 Hz, or 1,000-20,000 Hz) at two or more electrodes connected to the same system. The instructions for use may also include instructions for using variable stimulating frequencies (e.g., concurrent, simultaneous, alternating, in a pattern, overlapping, non-overlapping, randomly, interleaved, and the like) with two or more electrodes connected to the same system. The instructions 58 can, of course, vary. The instructions 58 may be physically present in a kits holding the lead(s) 12 (as FIG. 22 shows) but can also be supplied separately. The instructions 58 can be embodied in separate instruction manuals, or in video or audio tapes, CD's, DVD's, or digital video or audio recordings. The instructions 58 for use can also be available through an internet web page.

To determine the optimal placement for the lead(s) 12, test stimulation may be delivered through needle electrodes, and muscle responses may be observed. The motor point (s) of the target muscle(s) may be located first in order to confirm that the muscles are innervated. Needle electrodes may be used because they can be easily repositioned until the optimal location to deliver stimulation is determined.

At least one electrode may be placed in muscle tissue at a therapeutically effective distance spaced from a targeted nerve or nerve branch. By a “therapeutically effective distance” is meant that the electrode is not placed against the targeted nerve, but rather spaced therefrom, electrically coupled to the nerve through other bodily tissue. The spacing is advantageous because it simplifies placement and stimulation procedures, reduces the risk of neurological injury to the patient, shortens the procedure time, makes the method of pain relief more robust and durable and less likely to fail or lose effectiveness over time. Such placement also allows the electrode(s) to be placed in tissue more resistant to electrode migration or unwanted movement and more tolerant of motion and short and/or long-term changes in electrode position relative to the targeted nerve. The lead(s) may be inserted via the introducer in conventional fashion, which may be similar in size and shape to a hypodermic needle. The introducer 30 may be any size. In a preferred embodiment, the introducer 30 may range in size from 17 gauge to 26 gauge. Prior to inserting the introducer 30, the insertion site may be cleaned with a disinfectant (e.g., Betadine, 2% Chlorhexidine/80% alcohol, 10% povidone-iodine, or similar agent). A local anesthetic(s) may be administered topically and/or subcutaneously to the area in which the electrode and/or introducer will be inserted.

The position of the electrodes may be checked by imaging techniques, such as ultrasound, fluoroscopy, or X-rays. Following placement of the lead(s), the portion of the lead(s) which exit the skin may be secured to the skin using covering bandages and/or adhesives.

Electrical stimulation may be applied to the targeted nerve or nerve branch during and after placement of the electrode to determine whether stimulation can generate comfortable sensations or paresthesias that overlap with the region of pain and/or reduce pain. The pain may be perceived to be contained within a specific part(s) of the body and/or it may be perceived to be located outside of the body, as may be the case in persons with amputations who have phantom limb pain or pain in the amputated (or phantom) limb(s).

In a percutaneous system 10 (as FIGS. 21A to 21D show), the leads 12 may be percutaneously placed near the targeted nerve or nerve branch and exit at a skin puncture site 16. A trial or screening test may be conducted in a clinical setting (e.g., an office of a clinician, a laboratory, a procedure room, an operating room, etc.). During the trial, the lead is coupled to an external pulse generator 28 and temporary percutaneous and/or surface return electrodes, to confirm paresthesia coverage and/or pain relief of the painful areas.

If the clinical screening test is successful, the patient may proceed to a home-trial coupled to an external pulse generator 28 (as shown in FIG. 21C) and temporary percutaneous and/or surface return electrodes, to determine if pain relief can be sustained in the home environment. The trial period may range from minutes to hours to days to weeks to months. The preferred trial period may be between 3 and 21 days.

If either the screening test or home trial is unsuccessful, the leads 12 may be quickly and easily removed. However, if the screening test and/or home-trial are successful, the patient's percutaneous system may be converted into a fully implanted system (as shown in FIG. 21D) by replacing the external pulse generator with an implantable pulse generator 28 (the housing of which serves as a return electrode).

Alternatively, it may be preferred to use a percutaneous system(s) as a therapy without proceeding to a fully implantable system. It is also to be appreciated that a home-trial is not a requirement for either the percutaneous system or a fully implanted system.

The duration of therapy for a percutaneous system may range from minutes to days to weeks to months to multiple years, but a preferred embodiment includes a duration ranging from 1 to 12 weeks.

Electrical stimulation is applied between the lead and return electrodes (uni-polar mode). Regulated current is the preferred type of stimulation, but other type(s) of stimulation (e.g., non-regulated current such as voltage-regulated) may also be used. Multiple types of electrodes may be used, such as surface, percutaneous, and/or implantable electrodes. The surface electrodes may be a standard shape or they may be tailored if needed to fit the contour of the skin.

In a preferred embodiment of a percutaneous system, the surface electrode(s) may serve as the anode(s) (or return electrode(s)), but the surface electrode(s) may be used as the cathode(s) (active electrode(s)) if necessary. When serving as a return electrode, the location of the electrode(s) is not critical and may be positioned anywhere in the general vicinity, provided that the current path does not cross the heart. If a surface electrode(s) serves as an active electrode(s), it (they) may be positioned near the target stimulation area(s) (e.g., on the skin surface over the target nerve or nerve branch).

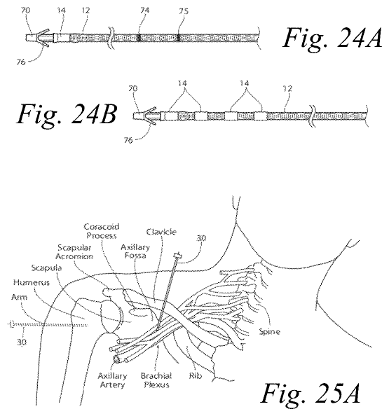

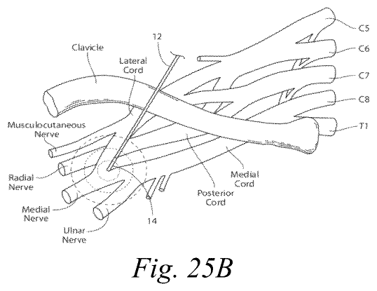

The electrode lead may be placed via multiple types of approaches. In one embodiment, the approach may be similar to needle placement for electromyography (EMG). For example (as shown in FIG. 25A), if the targeted nerve or nerve branch includes nerves of the brachial plexus, the approach can include:
1) Place the patient in a comfortable and/or appropriate position with head turned away from the lead insertion site.
2) Prepare the lead insertion site with antiseptic and local subcutaneous anesthetic (e.g., 2% lidocaine).
3) Locate the site of skin puncture with appropriate landmarks, such as the clavical, coracoid process, and/or axilla, as necessary.
4) Insert one or more sterile percutaneous electrode leads 12 preloaded in the introducer needle 30 at a predetermined angle based on landmarks used.
5) Place a surface stimulation return electrode in proximity of the area in which the percutaneous lead 12 has been placed. Test stimulation will be applied to the lead(s) 12, with the surface electrode providing a return path. The surface electrode may be placed adjacent to the lead. Its position is not critical to the therapy and it can be moved throughout the therapy to reduce the risk of skin irritation.
6) Couple the lead(s) 12 to the external pulse generator 28 and to the return electrode. Set the desired stimulation parameters. Test stimulation may be delivered using a current-regulated pulse generator, for example. The external pulse generator 28 may be programmed to about 0.1 to about 30 milliamps (mA), a pulse duration or width of about 10 to about 1000 microseconds (μs), a pulse frequency of about 1 to about 20000 Hertz (Hz), and a preferred on-off duty cycle of about 25 to about 90 percent (on vs. off), as a non-limiting example. Alternatively, rather than have an on-off duty cycle, the stimulation can be delivered constantly for a predetermined treatment time, such as about two to eight weeks. The external pulse generator may be programmed to differing levels of current, pulse duration, pulse frequency, and/or duty cycle for each lead and/or for each electrode.
7) Advance the introducer slowly until the patient reports the first evoked sensation in the region experiencing pain. Progressively reduce the stimulus amplitude and advance the introducer more slowly until the sensation can be evoked in the painful region at a predetermined stimulus amplitude (e.g., about 0.1-5.0 mA). Stop the advancement of the introducer, and increase the stimulus amplitude in small increments (e.g., about 0.1-0.5 mA) until the stimulation-evoked comfortable sensation (e.g., a tingling paresthesia, muscle contraction, and/or pressure) expands to overlay the entire region of pain.
8) Withdraw the introducer 30, leaving the percutaneous lead(s) 12 in proximity but away from the target nerve (see FIG. 25B).
9) Cover the percutaneous exit site and lead 12 with a bandage. A bandage may also be used to secure the external portion of the lead 12 (or an extension cable used to couple the lead 12 to the external pulse generator) to the skin. It is expected the length of time to place the lead 12 to be less than 10 minutes, although the process may be shorter or longer.
10) For each lead and/or electrode, vary the stimulus amplitude in small steps (e.g., 0.1-0.5 mA) to determine the thresholds at which stimulation evokes first sensation (TsEN), sensation (tingling paresthesia, muscle contraction, and/or pressure) superimposed on the region of pain (Tsup), muscle twitch (TMusl) of the target muscle (innervated or not innervated by the target nerve), and/or maximum comfortable sensation (Tx). Query the patient at each stimulus amplitude to determine sensation level, and visually monitor muscle response. Record the results.
11) It is possible that stimulation intensities may need to be increased slightly during the process due to causes such as habituation or the patient becoming accustomed to sensation, but the need for increased intensity is unlikely and usually only occurs after several days to weeks to months as the tissue encapsulates and the patient accommodates to stimulation. It is to be appreciated that the need for increased intensity could happen at any time, even years out, which would likely be due to either lead migration or habituation, but may also be due reasons ranging from nerve damage to plasticity/reorganization in the central nervous system.
12) If paresthesias or comfortable sensations cannot be evoked with the initial lead and/or electrode placement, redirect the introducer 30.
13) If paresthesias or comfortable sensations still cannot be evoked in a given patient, then the muscle twitch response of the muscle innervated or not innervated by the target nerve may be used to guide lead placement and then increase stimulus intensity until sufficient paresthesias or comfortable sensations are elicited in the painful region. Minimal muscle contraction may be acceptable if it is well tolerated by the patient in exchange for significant pain relief and if it does not lead to additional discomfort or fatigue.
14) If stimulation evokes muscle contraction at a lower stimulus threshold than paresthesias or comfortable sensations (e.g., if TMus Tsup) and contraction leads to discomfort, then a lower stimulus frequency (e.g., 12 Hz or 1-20, 2-18, 4-16, 6-16, 8-14, 8-16, 10-16, 10-14, 10-12, 12, 4-20 Hz) may be used because low frequencies (e.g., 4-20 Hz) have been shown to minimize discomfort due to muscle contraction and provide >50% relief of shoulder pain in stroke patients while still inhibiting transmission of pain signals in the central nervous system in animals. If continued muscle contraction leads to pain due to fatigue, change the duty cycle, using parameters shown to reduce muscle fatigue and related discomfort in the upper extremity (e.g., 5 s ramp up, 10 s on, 5 s ramp down, 10 s off). As a non-limiting example, the stimulation may be cycled through multiple periods or phases, such as a:
   a. first phase that is an increasing or inclining phase or period (e.g., increase or ramp up of intensity (e.g., amplitude and/or pulse duration) over a period of time (e.g., 1, 2, 3, 4, 5, 6, 7, 8, 9, 10, or over a range such as 1-5, 1-10, 1-15, or 1-20 seconds)
   b. second phase that is an stable, flat, plateau, and/or unchanging phase or period (e.g., “on” and consistent intensity (e.g., consistent amplitude and/or pulse duration) over a period of time (e.g., 1, 2, 3, 4, 5, 6, 7, 8, 9, 10, 11, 12, 13, 14, 15, 16, 17, 18, 19, 20, or over a range such as 1-5, 1-10, 1-15, 1-20, 1-25, 1-30, 1-35, 1-40, 1-45, 1-50, 1-55, or 1-60 seconds)
   c. third phase that is an decreasing or declining phase or period (e.g., decreasing or ramp down of intensity (e.g., amplitude and/or pulse duration) over a period of time (e.g., 1, 2, 3, 4, 5, 6, 7, 8, 9, 10, or over a range such as 1-5, 1-10, 1-15, or 1-20 seconds)
   d. fourth phase that is an stable, flat, plateau, and/or unchanging phase or period (e.g., low or “off” and consistent intensity (e.g., consistent amplitude and/or pulse duration) which may be low or zero (0) over a period of time (e.g., 1, 2, 3, 4, 5, 6, 7, 8, 9, 10, 11, 12, 13, 14, 15, 16, 17, 18, 19, 20, or over a range such as 1-5, 1-10, 1-15, 1-20, 1-25, 1-30, 1-35, 1-40, 1-45, 1-50, 1-55, or 1-60 seconds)
15) If stimulation fails to elicit paresthesias or comfortable sensations in all areas of pain, then additional percutaneous leads with one or more electrodes (not shown) may need to be placed to stimulate the nerves that are not activated by the first lead 12.
16) If stimulation is successful, e.g., if the screening test and/or home-trial are successful, the patient's percutaneous system (see FIG. 21) may be converted into a fully implanted system by replacing the external pulse generator 28 with an implantable pulse generator that is implanted in a convenient area (see FIG. 21D) (e.g., in a subcutaneous pocket over the hip or in the subclavicular area). In one embodiment, the electrode leads 12 used in the screening test and/or home-trial may be totally removed and discarded, and new completely implantable leads may be tunneled subcutaneously and coupled to the implantable pulse generator. In an alternative embodiment, two part leads may be incorporated in the screening test and/or home-trial where the implantable parts are completely under the skin and connected to a percutaneous connector (i.e., extension) that can be discarded after removal. The implantable parts may then be tunneled and coupled to the implantable pulse generator, or a new sterile extension may be used to couple the lead to the implantable pulse generator.

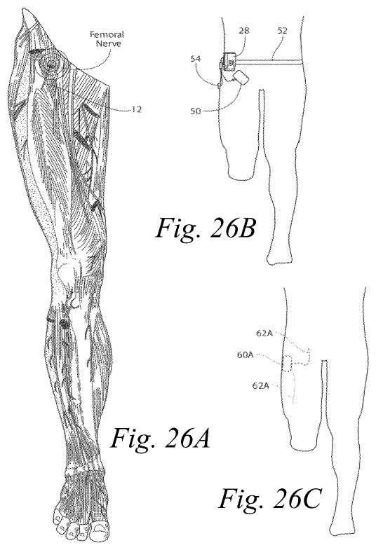

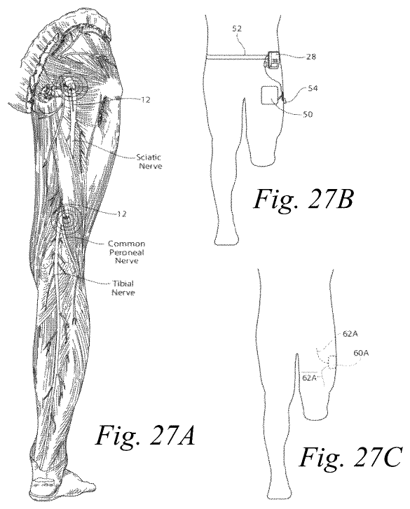

Alternatively, when the targeted nerve or nerve branch includes one or more nerves of the lumbar plexus or sacral plexus, the approach may be either a posterior (shown in FIG. 26A) or an anterior approach (shown in FIG. 27A), similar to those used for regional anesthesia of the same targeted nerve, except that the approach is used for placement through an introducer of stimulation lead(s) or electrode(s) in electrical proximity to but spaced away from a nerve or nerve branch, and not for chemically-induced regional anesthesia. Unlike regional anesthesia, the approach to nerves of the lumbar plexus or sacral plexus do not involve the application of anesthesia to the nerve, and, when the introducer is withdrawn, the lead(s) or electrode(s) may be left behind to provide the desired stimulation of the target nerve or nerve branch.

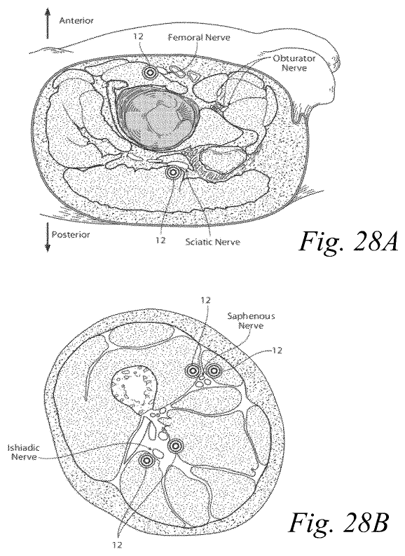

For example, when the targeted nerve or nerve branch includes the sciatic nerve (see FIG. 28A), the introducer(s) 30 and/or lead(s) 12 may be directed towards the sciatic nerve using a posterior approach, such as the transgluteal approach or subgluteal approach.

Alternatively, an adapted approach may also be used to minimize patient discomfort or damages or complications related to the therapy and/or system. As an example, the introducer(s) and/or lead(s) may be inserted from a more lateral insertion site (or another site that is more desirable) than is typically used for regional anesthesiology or other approaches because it has been found that a more lateral (or other) insertion site minimizes patient discomfort. The insertion site and/or path may be adapted or an alternative insertion site and/or path may be selected with an understanding of the type of tissue, muscle and other tissue planes, fascicular and/or fascial compartments, innervation of the tissue, muscle orientation, pennation angle, muscle fiber directionality, vascular and/or lymphatic vessels and structures, and other considerations below, surrounding, or near the insertion site or path. As non-limiting examples, it may be desirable to minimize the number of muscle planes that are transversed or crossed with the introducer(s) and/or lead(s), or it may be desirable to use an insertion site and/or insertion path that is not densely innervated by non-target sensory nerve fibers, maximizing the comfort of the placement procedure and the therapy following removal of the introducer, or it may be desirable to orient the direction of the introducer(s) and/or lead(s) relative to the directionality of the muscle fibers, placing the lead(s) in line with (e.g., parallel to) or orthogonal to the muscle fibers or any variation between parallel or orthogonal to the muscle fibers, or it may be desirable for the insertion path to necessitate greater length of lead under the skin to minimize the impact of lead migration and maximize therapy. In this embodiment, the robust neural responses generated by multimodal stimulation through multiple electrodes minimizes susceptibility to lead migration (or other interruptions in treatment) with a single mode of stimulation, increasing the likelihood for a patient to experience successful pain relief.

This approach allows lead placement near a targeted nerve or nerve branch with a simple, quick (e.g., less than 10 minutes) outpatient procedure that may be performed in a standard community-based clinic. This makes possible widespread use and provides a minimally-invasive screening test to determine if patients will benefit from the device before receiving a fully implanted system.

The landmarks for the transgluteal approach may include the greater trochanter and the posterior superior iliac spine. The introducer 30 may be inserted distal or proximal (e.g., approximately up to about 12 cm in a preferred embodiment) and/or medial or lateral (e.g., approximately up to about 12 cm in a preferred embodiment) to the midpoint between the greater trochanter and the posterior iliac spine. Alternatively, the introducer 30 may be inserted at the midpoint between the greater trochanter and the posterior iliac spine. As a non-limiting example of patient positioning, the patient may be in a lateral decubitus position and tilted slightly forward in a preferred embodiment.

The landmarks for the subgluteal approach may include the greater trochanter and the ischial tuberosity. The introducer may be inserted distal or proximal (e.g., approximately up to about 12 cm in a preferred embodiment) and/or medialorlateral (e.g., approximately up to about 12 cm in a preferred embodiment) to the midpoint between the greater trochanter and the ischial tuberosity. Alternatively, the introducer 30 may be inserted at the midpoint between the greater trochanter and the ischial tuberosity.

For any approach targeting the sciatic nerve (e.g., the transgluteal, subgluteal, and/or another or adapted approach), it may be beneficial to insert the introducer lateral to the midpoint between the relevant landmarks. A more lateral insertion point may maximize safety to the patient and/or the system, and it may increase patient comfort and minimize risk of damage to the lead or migration of the lead. As a non-limiting example of lateral placement, the introducer may be inserted lateral to the midpoint between the greater trochanter and the ischial tuberosity. The introducer may be inserted anywhere between the midpoint and the greater trochanter. The introducer may be inserted proximal or distal to the line between the greater trochanter and ischial tuberosity. It may be beneficial to insert the introducer distal to this line.

For example, when the targeted nerve includes the femoral nerve (see FIG. 28A), percutaneous leads 12 may be directed towards the femoral nerve using an anterior approach. The landmarks may include the inguinal ligament, inguinal crease, and femoral artery. The patient may be in the supine position with ipsilateral extremity slightly (approximately 10 to 20 degrees) abducted. The introducer may be inserted near or below the femoral or inguinal crease and approximately 1 cm or more lateral to the pulse of the femoral artery. More detail on placement of a percutaneous lead 12 for stimulation of the femoral nerve may be found below.

The size and shape of tissues, such as the buttocks, surrounding the target nerves may vary across patients, and the approach may be modified as needed to accommodate various body sizes and shapes to access the target nerve.

In non-amputee patients, introducer placement can be often guided by muscle response to electrical stimulation, but the muscle response may not be available in amputees, or may not be available and/or be unreliable in other situations (e.g., a degenerative diseases or condition such as diabetes of impaired vascular function in which the nerves are slowly degenerating, progressing from the periphery, or due to trauma).

In these situations, placement may be guided by the individual's report of stimulus-evoked sensations (e.g., paresthesias) as the introducer is placed during test stimulation. Additionally, the response of remaining muscles to stimulation may also be used to guide placement of the introducer and lead(s) and/or electrode(s).

As shown in FIG. 28B, more than a single lead 12 may be placed around or in the vicinity of a given nerve or nerve branch, using either an anterior approach (e.g., femoral nerve) or a posterior approach (e.g., sciatic nerve). As FIG. 10A, B, and C show, one or more leads 12 can be placed at different superior-inferior positions along one nerve or nerve branch and/or along different nerves and their branches.

As FIG. 26B (anterior approach, e.g., femoral nerve) and 27B (posterior approach, e.g., sciatic nerve) show, the lead 12 can be coupled to an external pulse generator 28 worn, e.g., on a belt 52, for a trial or temporary stimulation regime. In this arrangement, the leads 12 are covered with a bandage 50, and a surface electrode 54 serves as a return electrode. The external/percutaneous system shown in FIGS. 26B and 27B may be replaced by an implanted system using an implanted pulse generator 60 and intramuscular and/or adipose and tunneled leads 62A, as shown in FIGS. 26C and 27C, respectively. In this arrangement, the case of the implanted pulse generator 60A comprises the return electrode.

### D. Stimulation Parameters

Control of the stimulator and stimulation parameters may be provided by one or more external controllers. In the case of an external stimulator, the controller may be integrated with the external stimulator. The implanted pulse generator external controller (e.g., clinical programmer) may be a remote unit that uses RF (Radio Frequency) wireless telemetry communications (rather than an inductively coupled telemetry) to control the implanted pulse generator. The external or implantable pulse generator may use passive charge recovery to generate the stimulation waveform, regulated voltage (e.g., 10 mV to 20 V), and/or regulated current (e.g., about 10 μA to about 50 mA). Passive charge recovery is one method of generating a biphasic, charge-balanced pulse as desired for tissue stimulation without severe side effects due to a DC component of the current.

The neurostimulation pulse may by monophasic, biphasic, and/or multi-phasic. In the case of the biphasic or multi-phasic pulse, the pulse may be symmetrical or asymmetrical. Its shape may be, but is not limited to rectangular or exponential or sinusoidal or a combination of rectangular, exponential, and/or sinusoidal waveforms. The pulse width of each phase may range between e.g., about 0.1 μsec. to about 1.0 sec., as non-limiting examples. In a preferred embodiment, the neurostimulation waveform is cathodic stimulation (though anodic will work), biphasic, and asymmetrical.

Pulses may be applied in continuous or intermittent trains (i.e., the stimulus frequency changes as a function of time). In the case of intermittent pulses, the on/off duty cycle of pulses may be symmetrical or asymmetrical, and the duty cycle may be regular and repeatable from one intermittent burst to the next or the duty cycle of each set of bursts may vary in a pre-programmed manner or vary in a random (or pseudo random) fashion. Stimulation that modulates the sensory and/or motor sensations of the patient may assist in preventing loss of efficacy due to innate processes of neural plasticity such as habituation or accommodation by varying the stimulus frequency and/or duty cycle.

The stimulating frequency may range from e.g., about 1 Hz to about 20000 Hz (e.g., 0.5-12 Hz or 0.1-20 Hz, or 0.05-40 Hz, 20-120 Hz, or 12-200 Hz, or 200-500 Hz, or 500-15,000 Hz, or 1,500 Hz, or 5,000 Hz, or 15,000 Hz, or 20,000 Hz, or 1,000-20,000 Hz), and the frequency of stimulation may be constant or varying. In the case of applying stimulation with varying frequencies, the frequencies may vary in a consistent and repeatable pattern or in a random (or pseudo random) fashion, or in non-repeating, preprogrammed patterns, or a combination of repeatable, random (or pseudo random) patterns, and/or non-repeating pregrommed patterns.

In a representative non-limiting embodiment, the stimulator is set to an intensity (e.g., 0.1-20 mA or 0.05-40 mA, or 0.01-200 mA), a pulse duration or width 1-300 μs (or 5-1000 μs, or 1-10,000 μs) sufficient to activate the targeted nerve or nerve branch at some therapeutically effective distance (e.g., 1 mm, or 1-5 mm, or 5 mm, or 5-10 mm, or 10 mm, or 10-20mm or 20 mm) away from the targeted nerve(s) or nerve branch(es). If the stimulus intensity is too great, it may generate muscle twitch(es) or contraction(s) sufficient to disrupt correct placement of the lead. If stimulus intensity is too low, it may be an indication that the lead has been advanced too close to the targeted nerve or nerve branch (e.g., beyond the optimal position), possibly leading to incorrect guidance, nerve damage, mechanically evoked sensation due to direct interaction between the stimulating probe or lead and the target nerve (e.g., pain and/or paresthesia and/or uncomfortable sensations and/or muscle contraction), inability to activate the target nerve fiber(s) without activating non-target nerve fiber(s), improper placement, and/or improper anchoring of the lead (e.g., the lead may be too close to the nerve and no longer able to anchor appropriately in the muscle tissue).

In a representative embodiment, the stimulator is set to a frequency (e.g., 0.5-12 Hz or 0.1-20 Hz, or 0.05-40 Hz) low enough to evoke visible muscle twitches (e.g., non-fused muscle contraction) and/or muscle contraction(s) of the targeted muscle(s) innervated by the target nerve or nerve branch, but high enough that that the targeted nerve or nerve branch will be activated before the lead is advanced beyond the optimal position.

In a representative embodiment, the stimulator is set to one frequency (e.g., 0.5-12 Hz or 0.1-20 Hz, or 0.05-40 Hz) low enough to evoke visible muscle twitches (e.g., non-fused muscle contraction) and/or muscle contraction(s) of one or more targeted muscle(s) innervated by the target nerve and simultaneously set to a similar frequency (e.g., 0.5-12 Hz or 0.1-20 Hz, or 0.05-40 Hz) or one or more other frequencies (e.g., 20-120 Hz or 12-200 Hz or 200-500 Hz) high enough to evoke sensation(s) or comfortable paresthesia(s) in the region(s) of pain or in alternate target region(s) or one or more other frequencies (e.g., 500-20,000 Hz) high enough to produce neural effect(s) that may be sub-perception in the region(s) of pain or in alternate target region(s). In a representative embodiment, the stimulator is set to one frequency (mode) for one lead and the same or another frequency (another mode) for an additional lead. In an embodiment, a stimulator is set to n number of frequencies for n number of leads and/or electrodes.

In a representative embodiment, the stimulator is set to one frequency (e.g., 20-120 Hz, or 12-200 Hz) high enough to evoke sensation(s) or comfortable paresthesia(s) in the region(s) of pain and one or more other frequencies (e.g., 20-120 Hz or 12-200 Hz or 200-500 Hz) high enough to evoke sensation(s) or comfortable paresthesia(s) in the region(s) of pain or in alternate target region(s), or one or more other frequencies (e.g., 500-20,000 Hz) high enough to produce neural effect(s) that may be sub-perception in the region(s) of pain or in alternate target region(s).

As an alternative to using muscle twitch(es) or contraction(s) as indicator(s) of lead placement (distance from the nerve or nerve branch to electrode contact), patient sensation could instead be used to indicate lead location relative to the targeted nerve or nerve branch. Any combination of stimulus parameters that evoke sensation(s) may be used. Some stimulus parameters may evoke a more desirable response (e.g., more comfortable sensation), or a sensation that may be correlated with or specific to the specific target nerve fiber(s) within the targeted nerve or nerve branch.

As an example, other frequencies (e.g., 0.1-20 Hz, 20-100 Hz, 100-500 Hz, 500-15000 Hz, or 1000-20000 Hz) may evoke sensation(s) or comfortable paresthesia(s) in the region(s) of pain or in alternate target region(s) and may not be designed to evoke (and do not produce) a muscle contraction or a twitch from the present lead location. As another example, other frequencies (e.g., 0.1-20 Hz, 20-100 Hz, 100-500 Hz, 500-15000 Hz, or 1000-20000 Hz) may evoke sensation(s) or comfortable paresthesia(s) in the region(s) of pain or in alternate target region(s) and, by design or not by design, may produce a muscle contraction or a twitch from the present lead location that is difficult to observe visually without the use of EMG. In another example, the muscle contraction may be fused and no longer visually twitching, making it difficult to observe visually, but can be identified by EMG recording or other method (e.g., ultrasound visualization). To take advantage of potential indicator responses (e.g., muscle twitch and patient sensation), higher frequencies may be applied intermittently (at lower frequencies), where the higher frequencies (e.g., 20-35, 35-45, 45-60, 60-70, 70-85, 85-120, 20-120, 60-120, 96, 120, 120-200, 120-500, 200-500, 500-1500, 500-5000, 5000-15000, 20-15000 Hz) would normally cause fused muscle contraction if they were applied continuously but they are applied at an intermittent frequency (e.g., 0.5-4 Hz, or 4-20 Hz) that is low enough to allow the muscle to relax during the gaps between the bursts of stimulation, making it easier to visualize while still generating patient sensation at a higher frequency, allowing both muscle twitch and patient sensation to be used simultaneously as indicators of lead location relative to the targeted nerve or nerve branch.

While stimulation is being applied, the lead(s) (non-limiting examples of the lead(s) could include a single or multi-contact electrode that is designed for temporary (percutaneous) or long-term (implant) use or a needle electrode (used for in-office testing only)) may be advanced (e.g., slowly advanced) towards the targeted nerve or nerve branch until the desired indicator response (e.g., muscle twitch, muscle contraction, patient sensation, and/or some combination) is obtained at a first location X1. The intensity may then be decreased (e.g., gradually decreased) as the lead is advanced (e.g., advanced slowly) closer to the targeted nerve or nerve branch until the desired indicator response(s) may be obtained at smaller intensity(ies) within the target range (e.g., 0.1-30 mA (or 0.09-39 mA, or 0.009-199 mA), 1-300 us (or 5-1000 μs, or 1-10,000 μs)) at some distance (e.g., X2 mm, where X2<X1, and (as a non-limiting example) X1 may be multiple times larger than X2, such as X1 2*X2, or X15*X2, or X1 20*X2 from the target nerve. If specific response(s) (e.g., desired response(s) and/or undesired response(s)) can be obtained at a range of intensities that are too low, then the lead may be located in a non-optimal location (e.g., too close to the target nerve(s)) Non-limiting examples of ranges of intensities that may be considered too low include those that are a fraction (e.g., <⅔, or <⅕, or < 1/10) of the intensities that obtained the desired response(s) at X 1.

Additionally or alternatively, preferably with stimulation turned off, a needle electrode (e.g., an EMG monitoring electrode connected to a stimulator) may be inserted a predetermined distance, such as approximately to about 5.0 centimeters, and more preferably 0.5 to about 3.0 centimeters, from a target neural structure. The initial placement is preferably confirmed by the use of ultrasound or fluoroscopic imaging, including biological landmark identification. However, such imaging or landmark identification is not necessary. Once inserted to an initial position, a test stimulation may be delivered by the needle electrode at a desired intensity (e.g., a pulse duration or width of about 1 to about 200 μs, a frequency of about 0.1 to about 20000 Hz, and an amplitude of about 0.1 mA to about 30 mA delivered constantly). If no neurological response is reported by the patient and no neurological response is observed or detected by the clinician, the amplitude, pulse duration, and or frequency of the test stimulation may be increased or decreased until a neurological response is reported, observed, or detected.

A neurological response may be reported, observed, or detected at a location that is local to the stimulation delivery location (e.g., near the needle electrode). If a sensation is reported by the patient or a muscle contraction is observed at such location, the needle electrode may be too superficial and the electrode may be advanced further towards the target neural structure. In this case, the electrode is preferably advanced while the stimulation is turned off. Advancement may occur in the range of 0.1-2 cm, and the test stimulation process may be repeated. If a sensation is reported by the patient and the sensation is an uncomfortable sensation perceived to be originating at a point distal to the location of the electrode, or if a neurological response of muscle contraction is observed or detected as occurring distal to the electrode, it is an indication that the needle electrode is too close to the target neural structure, and the electrode is slightly withdrawn in the range of 0.1-2 cm while maintaining the needle electrode in vivo. During this slight withdrawal of the electrode, the stimulation is preferably off, and the test stimulation process may be repeated after electrode relocation. If in vivo readjustment or relocation of the electrode does not result in the goal of comfortable paresthesia in an area of pain, the needle electrode insertion trajectory and/or insertion site may be adjusted, and the process repeated. Once an appropriate electrode location has been determined, the depth and trajectory is noted and used for guiding and/or informing the insertion of the treatment stimulation electrode to be anchored for a predetermined treatment duration.

The preferred stimulus intensities are a function of many variables, are meant to serve as non-limiting examples only, and may need to be scaled accordingly. As an example, if electrode shape, geometry, or surface area were to change, then the stimulus intensities may need to change appropriately. For example, if the intensities were calculated for a lead with an electrode surface area of approximately 20 mm², then they may need to be scaled down accordingly to be used with a lead with an electrode surface area of 0.2 mm 2 because a decrease in stimulating surface area may increase the current density, increasing the potential to activate excitable tissue (e.g., target and non-target nerve(s) and/or fiber(s)). Alternatively, if the intensities were calculated for a lead with an electrode surface area of approximately 0.2 mm², then the intensities may need to be scaled up accordingly to be used with a lead with an electrode surface area of 20 mm². Alternatively, stimulus intensities may need to be scaled to account for variations in electrode shape or geometry (between or among electrodes) to compensate for any resulting variations in current density. In a non-limiting example, the electrode contact surface area may be 0.1-20 mm², 0.01-40 mm², or 0.001-200 mm². In a non-limiting example, the electrode contact configuration may include one or more of the following characteristics: cylindrical, conical, spherical, hemispherical, circular, triangular, trapezoidal, raised (or elevated), depressed (or recessed), flat, and/or borders and/or contours that are continuous, intermittent (or interrupted), and/or undulating.

Stimulus intensities may need to be scaled to account for biological factors, including but not limited to patient body size, weight, mass, habitus, age, and/or neurological condition (s). As a non-limiting example, patients that are older, have a higher body-mass index (BMI), and/or neuropathy (e.g., due to diabetes) may need to have stimulus intensities scaled higher (or lower) accordingly.

As mentioned above, if the lead is too far away from the targeted nerve or nerve branch, then stimulation may be unable to evoke the desired response (e.g., muscle contraction(s), comfortable sensation(s) (or paresthesia(s)), and/or pain relief) in the desired region(s) at the desired stimulus intensity(ies). If the lead is too close to the targeted nerve or nerve branch, then stimulation may be unable to evoke the desired response(s) (e.g., muscle contraction(s), comfortable sensation(s) (or paresthesia(s)), and/or pain relief) in the desired region(s) at the desired stimulus intensity(ies) without evoking undesirable response(s) (e.g., unwanted and/or painful muscle contraction(s), sensation(s) (or paresthesia(s)), increase in pain, and/or generation of additional pain in related or unrelated area(s)). In some cases, it may difficult to locate the optimal lead placement (or distance from the targeted nerve or nerve branch and/or it may be desirable to increase the range stimulus intensities that evoke the desired response(s) without evoking the undesired response(s) so alternative stimulus waveforms and/or combinations of leads and/or electrode contacts may be used. A non-limiting example of alternative stimulus waveforms may include the use of a pre-pulse to increase the excitability of the target fiber(s) and/or decrease the excitability of the non-target fiber(s).

Those skilled in the art will recognize that, for simplicity and clarity, the full structure and operation of all devices and processes suitable for use with the present system and method is not being depicted or described herein. Instead, only so much of a peripheral nerve stimulation system, e.g., including a lead(s), pulse generator, programmer, controller, power source, and/or supporting devices, hardware, software, firmware, external components, internal components, as is unique to the present system and method or necessary for an understanding of the present system and method is depicted and described. The remainder of the construction and operation of the system(s) described herein may conform to any of the various current implementations and practices known in the art.

### III. Representative Indications for Chronic or Temporary Pain Therapy

Localized pain in any area of the body (e.g., the skin, bone, joint, or muscle) can be treated by applying electrical stimulation to tissue (e.g., muscle, adipose, connective or other tissue) in electrical contact with but spaced from a targeted nerve or nerve branch. Electrical stimulation of nerves or nerve branches works by interfering with or blocking pain signals from reaching the brain, as FIGS. 11 and 12 schematically show.

Many pain indications can be treated by stimulation of nerves or their branches. Pain in the leg may occur in areas such as the thigh, calf, hip, shin, knee, foot, ankle, and toes. There may be multiple causes of leg pain, including but not limited to injury (e.g., traumatic) to a muscle, joint, tendon, ligament or bone; muscle or ligament damage; ligament sprain, muscle or tendon strain; disease or disorders; phlebitis, swelling, or inflammation; claudication; insufficient blood flow into (arterial insufficiency) or away from (venous insufficiency) a part of the leg or foot; ischemia; peripheral artery disease; arthritis; tumor (malignant or benign); peripheral neuropathy; diabetic peripheral neuropathy; and postherpetic neuralgia.

For example, peripheral artery disease can cause pain (especially during activity such as walking or running) because the effective narrowing of the arteries leads to a decrease in the supply of blood and therefore in the supply of nutrients such as oxygen to the active muscles, leading to pain. This phenomenon can occur in almost in area of the body but may be more common in the leg, especially parts of the lower leg, such as the calf. Activity is not always required to elicit pain and pain may occur even at rest (without activity or exercise). Nerve entrapment, compression, injury or other types of damage may cause pain in the areas innervated by the damaged nerve, which can lead to referred pain in an area distal to the injury.

For example, claudication pain (occurring in the calf muscle) could be treated by nerves or nerve branch stimulation by placing the lead in the gluteus muscle near the sciatic nerve, which passes by the gluteus muscle on its way to innervate the calf muscle.

In general pain due to poor blood flow to an area or damage to an area can be relieved by stimulation of the nerve innervating that area. Since diabetic neuropathy typically leads to pain in the more distal areas (toes/foot), stimulation of the sciatic nerve can relive that pain. Pain in the skin of the medial (inner) calf can be relieved by stimulation of the femoral nerve. Pain in the front of the thigh (quad's) can be relieved by stimulation of the femoral nerve. If pain overlaps more than one area, stimulation of multiple nerves (e.g., sciatic and femoral nerves) can be beneficial.

Stimulation of the intercostal nerves (originating from the Thoracic nerve roots (T1-12)) can relieve pain in regions innervated by the intercostal nerves such as pain from intercostal neuralgia or post herpetic neuralgia. The pain may be confined to the area (e.g., dermatomic area) innervated by 1 or 2 nerves and may follow outbreak (and recovery) of herpes zoster. The pain may last up to several months or years in some patients and may be caused by nerve irritation or damage due to herpes zoster.

Post-amputation (e.g., including residual limb and/or phantom limb) pain can also be treated by stimulating nerves or their branches. For example, upper extremity stimulation of nerves passing through the brachial plexus can relive residual limb pain and/or phantom limb pain that results from amputation of an upper limb. Likewise, lower extremity stimulation of nerves passing through the lumber plexus sacral plexus (e.g., the sciatic nerve or the femoral nerve) can relive residual limb pain and/or phantom limb pain that results from amputation of a lower limb.

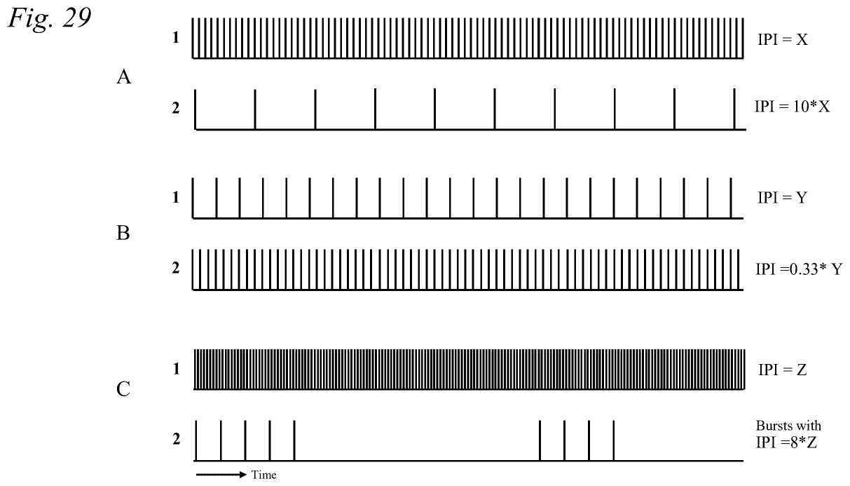

Described are systems and methods for applying electrical stimulation. In an embodiment, the systems and methods may include stimulating two or more electrodes on one or more leads at two or more modes (e.g., frequencies, patterns). In an embodiment, the systems and methods may include mixing modes (e.g., frequencies, patterns) of concurrent electrical stimulation at two or more electrodes or leads. For example, the systems and methods may include two or more electrodes and one or more leads and may include varying frequencies of stimulation for two or more electrodes. For example, the systems and methods may include more than one lead and may include varying frequencies of stimulation at one or more leads. In an embodiment, a lower stimulus frequency may be a subharmonic frequency of a highest stimulus frequency, wherein the subharmonic frequency is the highest stimulus frequency divided by an integer (e.g., the interpulse interval, IPI, of the lower frequency may be an integer multiple of the higher frequency's interpulse interval). The systems and methods may be used to provide electrical stimulation of a nerve or an innervated muscle and may be used to treat pain or provide other therapeutic benefits. The systems and methods may be used in percutaneous and fully implanted applications. Examples of harmonic frequencies are shown in FIG. 29A-C. It is appreciated that FIG. 29A-C does not portray an exhaustive list of harmonic frequencies.

For example, FIG. 29A-C illustrate non-exhaustive harmonic frequencies designed to provide stimulation at more than one frequency across more than one electrode. FIG. 29A illustrates a stimulation frequency applied at electrode 1 (inter-pulse interval, IPI=X; where frequency=1/IPI). A different stimulation frequency may be applied at electrode 2 designed using a subharmonic frequency wherein the IPI of the frequency applied to electrode 1 is multiplied by an integer (e.g., 10). In doing so, the stimulation frequency at electrode 1 is greater than the stimulation frequency at electrode 2, and the application of pulses may not overlap during concurrent stimulation using electrodes 1 and 2. FIG. 29B illustrates a stimulation frequency applied at electrode 1 (IPI=Y). A different stimulation frequency may be applied at electrode 2 designed using a harmonic frequency wherein the frequency applied to electrode 1 is divided by integer (e.g., 3). In doing so, the stimulation frequency at electrode 1 is lesser than the stimulation frequency at electrode 2, and the application of pulses may not overlap during concurrent stimulation using electrodes 1 and 2. FIG. 29C illustrates a stimulation frequency applied to electrode 1 (IPI=Z). A different frequency may be applied at electrode 2 designed using a subharmonic frequency wherein the frequency applied to electrode 1 is divided by an integer (e.g., 8) and applied in bursts or cycles (e.g., duty cycle<100%). In doing so, the stimulation frequency at electrode 1 is greater than the stimulation frequency at electrode 2, the stimulation pattern at electrode 2 may be used to evoke cyclical muscle contractions to avoid fatigue, and the application of pulses may not overlap during concurrent stimulation using electrodes 1 and 2.

The electrical stimulation system may include more than one source of electrical stimulation. In an embodiment, the electrical stimulation system may include two or more electrodes or leads. In an embodiment, the electrical stimulation system may include multiple or a plurality of electrodes or leads. In an embodiment, the electrical stimulation system may include at least two electrodes or leads. In an embodiment, the electrical stimulation system may include a first electrode or lead and a second electrode or lead. In an embodiment, the electrical stimulation system may include at least two stimulating electrodes (e.g., cathodes) and at least one return electrode (e.g., anodes). It is noted that other variations of in number of electrodes, cathodes and anodes, are also contemplated and may also be used without departing from this disclosure.

The two or more electrodes of the electrical stimulation system may be placed on an external surface of the skin, for example, as an external or percutaneous stimulator and/or the one or more electrodes of the electrical stimulation system may be surgically implanted as an implanted device. As examples, FIGS. 23B and 24B show a lead 12 including multiple electrodes 14. As shown in FIGS. 23B and 24B, lead 12 may include multiple electrodes 14 at or near a distal tip of the lead 12 (e.g., the distal-most electrode 14 may be at or near a distal tip of the lead 12 with additional electrodes 14 adjacent to or spaced apart from the preceding electrode 14 and away from the distal tip of the lead 12). It is noted that the multiple electrodes 14 may also be spaced apart from the distal tip of the lead 12 (e.g., the distal-most electrode 14 may be spaced apart from the distal tip of the lead 12 with additional electrodes 14 adjacent to or spaced apart from the preceding electrode 14 and away from the distal tip of the lead 12). As shown in FIGS. 23B and 24B, the lead 12 may include four electrodes 14 spaced apart from one another. It is noted that any number of electrodes 14 may also be used, including 2, 3, 4, 5, 6, 7, 8, 9, 10, 11, etc. electrodes 14 at a single lead 12. As described, multiple leads 12 having one or more electrodes 14 may also be used. As shown in FIGS. 23B and 24B, the electrodes 14 may be evenly spaced apart from one another (e.g., from the preceding electrode 14). It is also noted that the electrodes 14 may be spaced apart from one another at increasing lengths, decreasing lengths, at different lengths, etc.

The lead electrode 14 may also include, at or its distal tip, an anchoring element 48 such as that shown in FIG. 23B or anchoring element 70 such as that shown in FIG. 24B. As described, anchoring element 48 may take the form of a simple barb or bend. As described, anchoring element 70 may take the form of an array of shovel-like paddles or scallops 76 proximal to the proximal-most electrode 14. The anchoring elements 48, 70 may be sized and configured so that, when in contact with tissue, it takes purchase in tissue, to resist dislodgement or migration of the electrode out of the correct location in the surrounding tissue. In an embodiment, In addition, the lead 12 may include one or more ink markings 74, 75 (shown in FIGS. 23B and 24A) to aid the physician in its proper placement.

In an embodiment, some of the electrodes (e.g., one or more) may be used externally and some of the electrodes (e.g., one or more) may be implanted. In an embodiment, some of the stimulating electrodes (e.g., one or more) may be used externally and some of the stimulating electrodes (e.g., one or more) may be implanted. In an embodiment, all of the electrodes may be used externally or all of the electrodes may be implanted. In an embodiment, all of the stimulating electrodes may be used externally or all of the stimulating electrodes may be implanted.

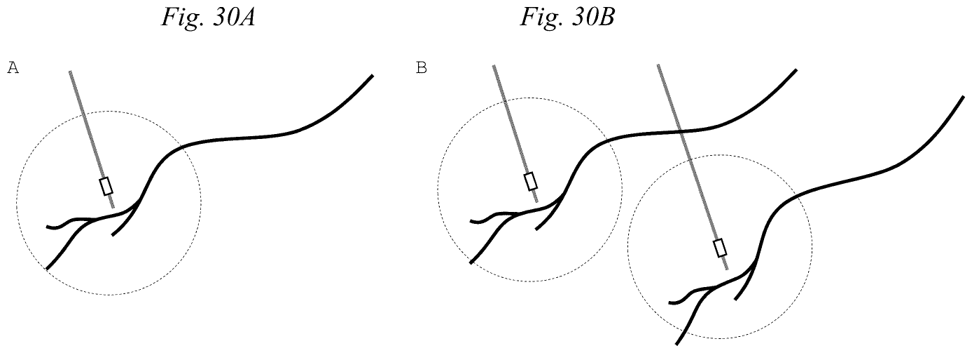

FIG. 30A is a schematic view showing an example for a system to apply unimodal stimulation (e.g., sensory stimulation alone or motor stimulation alone) from one electrode to provide stimulation to one nerve or nerve branch target. FIG. 30B is a schematic view showing an example for a system to apply unimodal stimulation (e.g., sensory stimulation alone or motor simulation alone) from two leads, each with one electrode, to provide stimulation to two nerve or nerve branch targets. The dotted circles around the electrodes represent stimulation waveforms or the area or zone of activation (e.g., area that is being activated).

The electrical stimulation system may further include more than one stimulating frequency. The electrical stimulation system may include at least two different stimulation frequencies. In an embodiment, the frequency may be variable within a range (e.g., 0.1-20 Hz, 20-100 Hz, 100-500 Hz, 500-15000 Hz, 1000-20000 Hz) and independently selectable for each nerve target (i.e., any combination of frequencies could be selected depending on nerve target and use). For example, the electrical stimulation system may provide stimulation at different frequency ranges (e.g., 0.1-20 Hz, 20-100 Hz, 100-500 Hz, 500-15000 Hz, 1000-20000) to two or more electrodes.

In an embodiment, the electrical stimulation system may include two or more electrodes providing stimulation at different frequencies. In an embodiment, the electrical stimulation system may include multiple or a plurality of electrodes providing stimulation of different frequencies. In an embodiment, the electrical stimulation system may include at least two electrodes each providing stimulation of different frequencies (e.g., 0.1-20 Hz, 20-100 Hz, 100-500 Hz, 500-15000 Hz, 1000-20000).

In an embodiment, the electrical stimulation system may include a first electrode and a second electrode, each providing stimulation of different frequencies (e.g., multimodal or bimodal stimulation, where one embodiment could include use of one mode of 4-20 Hz for one electrode and one mode could include use 60-120 Hz for a second electrode). In an embodiment, the electrical stimulation system may include a first electrode and a second electrode, each providing stimulation of different frequencies (e.g., multimodal stimulation, where one embodiment could include use of one mode of 4-20 Hz or 20-60 Hz or 60-120 Hz or 120-500 Hz or 500-15000 Hz or 1000-20000 Hz for one electrode and one mode could include use 4-20 Hz or 20-60 Hz or 60-120 Hz or 120-500 Hz or 500-15000 Hz or 1000-20000 Hz for a second electrode and one mode could include 4-20 Hz or 20-60 Hz or 60-120 Hz or 120-500 Hz or 500-15000 Hz for a third electrode and one mode could include 4-20 Hz or 20-60 Hz or 60-120 Hz or 120-500 Hz or 500-15000 or 1000-20000 Hz for a fourth electrode).

In another embodiment, the electrical stimulation system may include two or more electrodes, each providing stimulation of different frequency combinations (e.g., multimodal or bimodal stimulation with stimulation at a frequency between 500 -1500 Hz or 1000-20000 Hz for one mode and 1-500 Hz for the second mode). In another embodiment, the multimodal electrical stimulation delivered as multiple frequencies on multiple electrodes may each also incorporate cycling (e.g., patterns of on, off, decreasing, or increasing stimulation intensity values) rather than continuous stimulation.

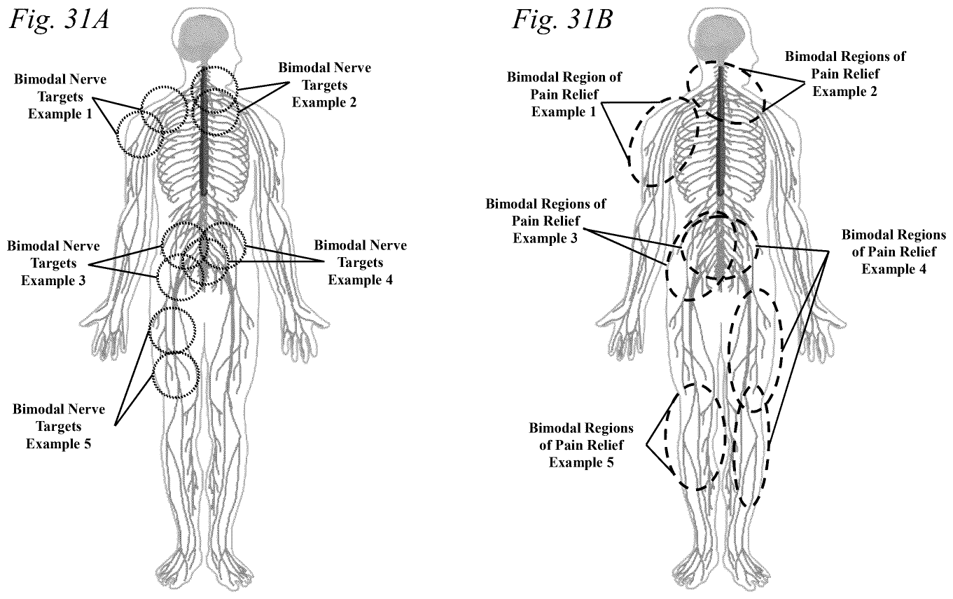

In an embodiment, one system containing two or more electrodes of differing frequencies (multiple modes, multimodal, bimodal) may be used in order to recondition the centrally maintained pain state. A non-exhaustive illustration of bimodal nerve targets are shown FIG. 31A, with examples of their potential regions of pain relief being shown in FIG. 31B.

For example, FIG. 31A is a schematic view showing non-limiting example nerve targets for a system to apply multimodal stimulation (e.g., motor stimulation and sensory stimulation) for the treatment of pain. Bimodal Nerve Targets Example 1 is showing example targets for a system that coordinates sensory stimulation of the suprascapular nerve and motor stimulation of the axillary nerve at the deltoid. Bimodal Nerve Targets Example 2 is showing example targets for a system that coordinates sensory stimulation of the occipital nerve(s) and motor stimulation of the cervical medial branch of the dorsal ramus. Bimodal Nerve Targets Example 3 is showing example targets for a system that coordinates sensory stimulation of the cluneal nerve and motor stimulation of the lumbar medial branch of the dorsal ramus. Bimodal Nerve Targets Example 4 is showing example targets for a system that coordinates sensory stimulation of the lumbar spinal nerve and motor stimulation of the lumbar medial branch of the dorsal ramus. Bimodal Nerve Targets Example 5 is showing example targets for a system that coordinates sensory stimulation of the femoral nerve and motor stimulation of the femoral nerve, distal at the vastus medialis oblique. FIG. 31B is a schematic view showing different example regions of pain relief provided by the example multimodal (e.g., bimodal) nerve targets illustrated in FIG. 31A.

It is appreciated that multimodal (e.g., bimodal) stimulation can be applied to nerve targets not illustrated on FIG. 31A and can treat pain regions not illustrated in FIG. 31B. The two or more frequencies provided over two or more electrodes may be used to activate neural fibers that engage multiple mechanisms to provide pain relief, resulting in a synergistic and/or complementary effect on neural pathways and central pain processing, and more comprehensive relief (e.g., providing pain relief of one or multiple etiologies of pain in one or multiple locations of pain). In an embodiment, the electrical stimulation system may include (i) one or more electrodes that provide stimulation at lower frequency(ies) (e.g., 1-20, 2-18, 4-16, 6-16, 8-14, 8-16, 10-16, 10-14, 10-12, 12, 4-20 Hz) to target motor nerve fibers (e.g., motor stimulation) in a motor nerve or mixed (e.g., motor and sensory) nerve that results in cyclical muscle twitches and/or muscle contractions and indirectly activate proprioceptive afferent pathways and (ii) one or more electrodes that provide stimulation at higher frequency(ies) (e.g., 120, 96, 60-70, 70-85, 85-100, 100-120, 60-120 Hz) to activate directly afferent pathways (e.g., sensory stimulation).

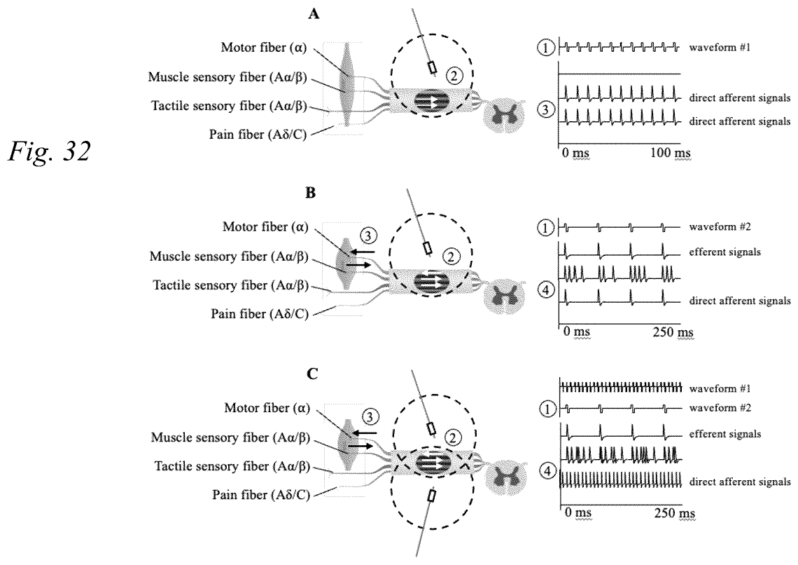

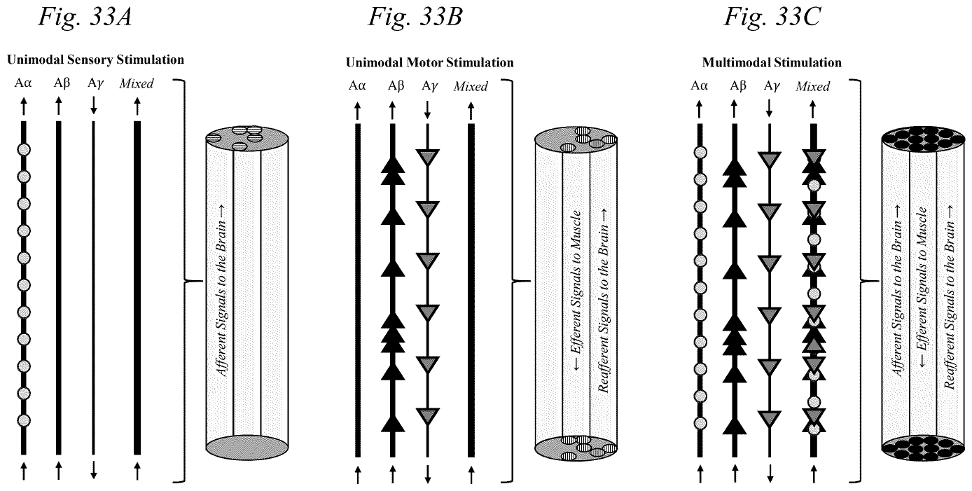

The different frequencies may be provided in various fashions to produce the synergistic effect, for example, simultaneously, concurrently, interleaved, and/or in another pattern in order to mimic the natural coordination between sensory and motor signals in the central nervous system, provide biomimetic signals (e.g., signals that match, mimic, or resemble natural sensations), or provide signals outside the realm of natural sensations in order to provide pain relief to systems or regions disrupted by disease, trauma, overuse, or otherwise subject to maladaptive changes. In an embodiment, multimodal stimulation (e.g., bimodal stimulation) may coordinate, combine, synchronize, and/or harmonize the unimodal sensory stimulation (e.g., continuous pulses at 100 Hz) providing direct afferent signals (FIGS. 32A and 33A) and unimodal motor stimulation (e.g., cyclic pulses at 12 Hz) providing efferent signals, indirect reafferent signals, and direct signals (FIGS. 32B and 33B) to create a more complex biomimetic signal for the treatment of pain (FIGS. 32C and 33C).

For example, FIG. 32A-C illustrate peripheral nerve stimulation approaches designed to selectively activate large diameter nerve fibers, where the dotted circles around the electrodes represent stimulation waveforms or the area or zone of activation (e.g., area that is being activated). FIG. 32A illustrates a system with unimodal sensory stimulation that delivers higher frequency stimulation via waveform #1 (e.g., continuous, 100 Hz) to selectively activate large diameter afferent nerves and produce comfortable sensations (e.g., paresthesia) in the region of pain. FIG. 32B illustrates a system with unimodal motor stimulation that delivers lower frequency stimulation via waveform #2 (e.g., cyclical, 12 Hz) to activate large diameter fibers (e.g., cutaneous afferents, muscle afferents, muscle efferents) and elicit activation of muscle afferent fibers and produce comfortable sensations (e.g., muscle contractions) in the region of pain. FIG. 32C illustrates a system with multimodal stimulation, which includes one or more electrodes that provides stimulation at lower frequency(ies) via waveform #2 (e.g., 4-20 Hz) to target motor nerve fibers (e.g., motor stimulation) in a motor nerve or mixed (e.g., motor and sensory) nerve that results in cyclical muscle twitches and/or muscle contractions and indirectly activate proprioceptive afferent pathways and one or more electrodes that provide stimulation at higher frequency(ies) via waveform #1 (e.g., 60-120 Hz) to activate directly afferent pathways (e.g., sensory stimulation).

The different frequencies may be provided in various fashions to produce the synergistic effect, for example, simultaneously, concurrently, interleaved, and/or in another pattern in order to mimic the natural coordination between sensory and motor signals in the central nervous system, provide biomimetic signals (e.g., signals that match, mimic, or resemble natural sensations), or provide signals outside the realm of natural sensations in order to provide pain relief to systems or regions disrupted by disease, trauma, overuse, or otherwise subject to maladaptive changes.

For example, FIG. 33A-C illustrate example signal propagation for afferent, efferent, and mixed nerves for a system providing unimodal sensory stimulation, unimodal motor stimulation, or multimodal (e.g., bimodal) stimulation. FIG. 33A illustrates unimodal sensory stimulation at higher frequencies (e.g., 40-120 Hz, 120-500 Hz), which elicits peripheral nerve afferent signaling to the brain. FIG. 33B illustrates unimodal motor stimulation at lower frequencies (e.g., 4-20 Hz) elicits peripheral nerve efferent signaling to muscle and reafferent peripheral nerve signaling to the brain (generation of both direct and indirect proprioceptive afferent signals). FIG. 33C illustrates multimodal stimulation, which coordinates motor, sensory, and mixed nerve responses and provides a greater volume of physiologically relevant peripheral nerve signals that mimic physiological signals in the central nervous system.

In an embodiment, multimodal stimulation may generate more intricate, complex, and/or effective signals and/or patterns of neural firing that are more effective than unimodal and/or physiological signals and/or patterns of neural firing (e.g., exemplary multimodal stimulation patterns in FIG. 33C illustrate more complex and voluminous afferent and efferent signaling compared to unimodal stimulation patterns in FIG. 33A-B) than can be programmed to be delivered directly by stimulation (e.g., via the combined activation of multiple nerves and/or patterns and including the generation of indirect proprioceptive afferent signals (e.g., reafferent signals), which travel together to provide superior neural signaling to more effectively recondition the underlying pain state for superior relief. This embodiment overcomes the challenge that the brain and pain state may be less likely to respond to externally generated (e.g., artificial) signals or patterns than internally generated (encoded) signals, and therefore the creation of more complex, quasi-physiological (e.g., biomimetic) neural signals with multimodal stimulation may more effectively reduce pain. In an embodiment, the brain and spinal cord neural networks responsible for processing pain signals are more responsive (i.e., plastic) and likely to change in response to the signals generated by multimodal stimulation compared to conventional unimodal stimulation. In this embodiment, the signals generated by the multimodal stimulation signal at two or more electrodes in the periphery produces changes in pain processing over a larger and more comprehensive cortical area. In this embodiment, the more robust signals (e.g., stronger intensity, larger volume of neural firing, more varied signal patterns that the brain is accustomed to receiving) generated by multimodal stimulation are more likely to produce changes in central pain processing in the brain because of the more robust signals generated.

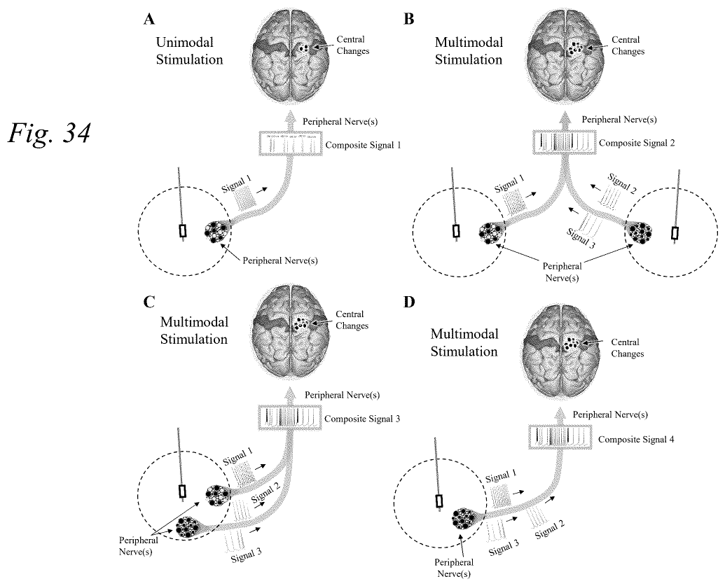

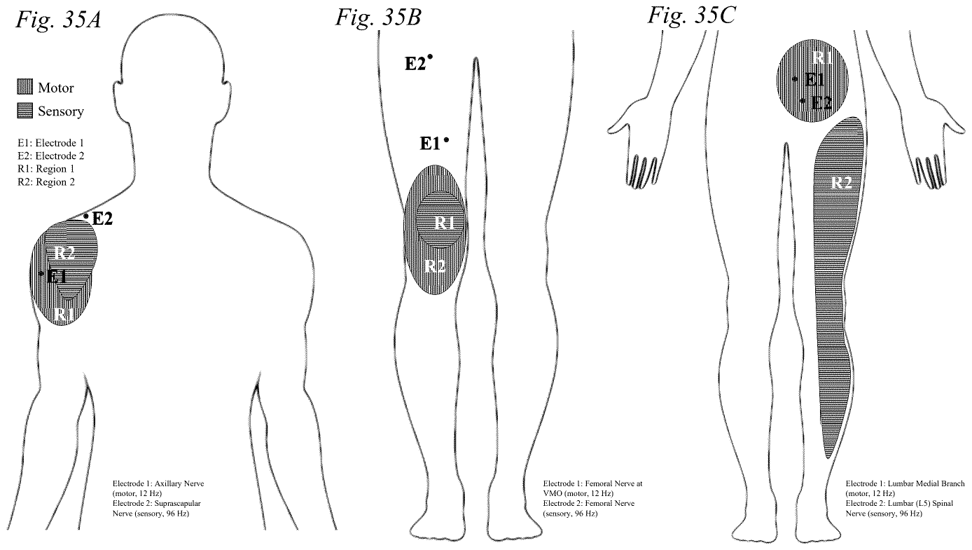

FIG. 34A illustrates an exemplary system providing unimodal sensory stimulation wherein one electrode provides a higher frequency stimulation (e.g., at 100 Hz, continuously), resulting in a composite 100 Hz continuous signal that produces central changes in the brain. FIG. 34B-D illustrates exemplary composite multimodal (e.g., bimodal) signals and respondent central changes.

For example, FIG. 34B illustrates an exemplary system providing bimodal (or multimodal) stimulation, wherein one lead with at least one electrode provides a higher frequency stimulation (e.g., at 96 Hz, continuously) to one nerve or nerve branch, resulting in afferent signaling along the peripheral nerves to the central nervous system and brain, and another lead with at least one electrode provides a lower frequency stimulation (e.g., 12 Hz, cyclic) to a different nerve or nerve branch, resulting in efferent signaling to muscle and corresponding reafferent peripheral nerve signaling to the central nervous system and brain, thus providing a composite peripheral nerve signal 2 that mimics physiological signals in the central nervous system and results in a greater volume of central changes in the brain.

For example, FIG. 34C illustrates an exemplary system providing bimodal (or multimodal) stimulation, wherein one lead contains one electrode that provides a higher frequency stimulation (e.g., at 96 Hz, continuously) to one peripheral nerve or peripheral nerve branch, resulting in afferent signaling to the brain, and another electrode on the same lead provides a lower frequency stimulation (e.g., 12 Hz, cyclic) to a different nerve or nerve branch, resulting in efferent signaling to muscle and corresponding reafferent signaling to the brain, thus providing a composite peripheral nerve signal 3 that mimics physiological signals transmitted to the central nervous system and results in a greater volume of central changes in the brain.

For example, FIG. 34D illustrates an exemplary system providing bimodal (or multimodal) stimulation, wherein one lead with one electrode provides a higher frequency stimulation (e.g., at 96 Hz, continuously) to one nerve or nerve branch, resulting in afferent signaling to the brain, and the same electrode on the same lead also provides a lower frequency stimulation (e.g., 12 Hz, cyclic) to the same nerve or nerve branch, resulting in efferent signaling to muscle and corresponding reafferent signaling along the peripheral nerves to the brain, thus providing a composite signal 4 that mimics signals in the central nervous system and results in a greater volume of central changes in the brain.

For each of FIG. 34A-D, the dotted circles around the electrodes represent stimulation waveforms or the area or zone of activation (e.g., area that is being activated).

Compared to unimodal stimulation of one nerve via one electrode (FIG. 34A), multimodal (e.g., bimodal) stimulation coordinating sensory stimulation of one nerve via one electrode (e.g., afferent signal 1) and motor stimulation of another nerve via another electrode (e.g., proprioceptive afferent signal 2 and reafferent signal 3) provides a more complex, biomimetic, quasi-physiologically relevant, and robust composite signal (FIG. 34B) to the central nervous system resulting in greater cortical changes which may more effectively reduce pain. Compared to unimodal stimulation of one nerve via one electrode (FIG. 34A), multimodal stimulation coordinating sensory stimulation of one nerve via one electrode (e.g., afferent signal 1) and motor stimulation of another nerve via the same electrode (e.g., proprioceptive afferent signal 2 and reafferent signal 3) provides a more complex, biomimetic, quasi-physiologically relevant, and robust composite signal (FIG. 34C) to the central nervous system resulting in greater cortical changes which may more effectively reduce pain. Compared to unimodal stimulation of one nerve via one electrode (FIG. 34A), multimodal stimulation coordinating sensory stimulation of one nerve via one electrode (e.g., afferent signal 1) and motor stimulation of the same nerve via the same electrode (e.g., proprioceptive afferent signal 2 and reafferent signal 3) provides a more complex, biomimetic, quasi-physiologically relevant, and robust composite signal (FIG. 34D) to the central nervous system resulting in greater cortical changes which may more effectively reduce pain, pain interference with function and/or activities of daily living, and/or thereby improve function, quality of life, patient satisfaction, and/or other symptoms.

In an embodiment, the system and method produce through stimulation from the two or more electrodes both (i) sensory nerve responses to provide a continuous paresthesia over the region of pain and (ii) periodic, cyclical, cycled, intermittent, and/or continuous motor nerve response to elicit a comfortable muscle twitch and/or contraction without unwanted fatigue. The two or more electrodes of differing frequencies may be placed near the same nerve or nerve branch, engaging in both direct afferent and indirect afferent (e.g., reafferent) pathways via multimodal (e.g., complementary) application of motor stimulation and sensory stimulation. In an embodiment, one system containing two or more electrodes with two or more frequencies may be permanently implanted in order to provide the aforementioned continuous inputs that recondition the centrally maintained pain state long-term.

FIG. 35A-C are non-limiting examples of multimodal (e.g., bimodal) applications of motor and sensory stimulation to provide robust stimulation coverage and relief of pain in multiple regions of the body. Electrode placement for nerve target stimulation may be within the region of pain and desired area of coverage, completely outside the region of pain, or mixed. The location of sensations generated by stimulation and areas of pain relief generated by multimodal (e.g., bimodal) stimulation may be either partially overlapping, fully overlapping, or no overlap.

FIG. 35A is a schematic view of multimodal (e.g., bimodal) stimulation that coordinates cyclic motor stimulation of the axillary nerve (Electrode 1, E1) at a lower frequency (e.g., 12 Hz) to treat region of pain 1 (R1) and continuous sensory stimulation of the suprascapular nerve (Electrode 2, E2) at a higher frequency (e.g., 96 Hz) to treat region of pain 2 (R2), as an application for the synergistic treatment of multiple etiologies of shoulder pain. As shown in FIG. 35A, the electrical stimulation system may coordinate the delivery of motor stimulation (e.g., cyclically with a frequency of 12 Hz) at the axillary nerve at the deltoid via one electrode and sensory stimulation (e.g., continuously at 96 Hz) at the suprascapular nerve via another electrode, thus providing a complex combinatory signal that overlaps one or more than one region of pain in the shoulder.

FIG. 35B is a schematic view of multimodal (e.g., bimodal) stimulation that coordinates cyclic motor stimulation of the femoral nerve at the vastus medialis oblique (Electrode 1, E1) at a lower frequency (e.g., 12 Hz) to treat region of pain 1 (R1) and continuous sensory stimulation of the femoral nerve (Electrode 2, E2) at a higher frequency (e.g., 96 Hz) to treat region of pain 2 (R2), as an application for the synergistic treatment of multiple etiologies of knee pain. As shown in FIG. 35B, the electrical stimulation system may coordinate the delivery of motor stimulation (e.g., cyclically with a frequency of 12 Hz) at the femoral nerve, distal at the at the vastus medialis oblique via one electrode and sensory stimulation (e.g., continuously at 96 Hz) at the femoral via another electrode, thus providing a complex combinatory signal that overlaps one or more than one region of pain in the knee. It is appreciated that one, more than one, or all of the electrode's placement for nerve target stimulation may be within the region of pain and desired area of comfortable stimulation, or may be completely outside of the region of pain and desired area of comfortable stimulation, or may only be partially inside or outside the region of pain and desired area of comfortable stimulation.

FIG. 35C is a schematic view of multimodal (e.g., bimodal) stimulation that coordinates cyclic motor stimulation of the lumbar medial branch of the dorsal ramus (Electrode 1, E1) at a lower frequency (e.g., 12 Hz) to treat region of pain 1 (R1) and continuous sensory stimulation of the lumbar spinal nerve at L5 (Electrode 2, E2) at a higher frequency (e.g., 96 Hz) to treat region of pain 2 (R2), as an application for the synergistic treatment of multiple etiologies of radicular back and leg pain. As shown in FIG. 35C, the electrical stimulation system may coordinate the delivery of motor stimulation (e.g., cyclically with a frequency of 12 Hz) at lumbar medial branch of the dorsal ramus via one electrode and sensory stimulation (e.g., continuously at 96 Hz) at the lumbar spinal nerve via another electrode, thus providing a complex combinatory signal that overlaps one or more than one region of pain in the low back and/or legs.

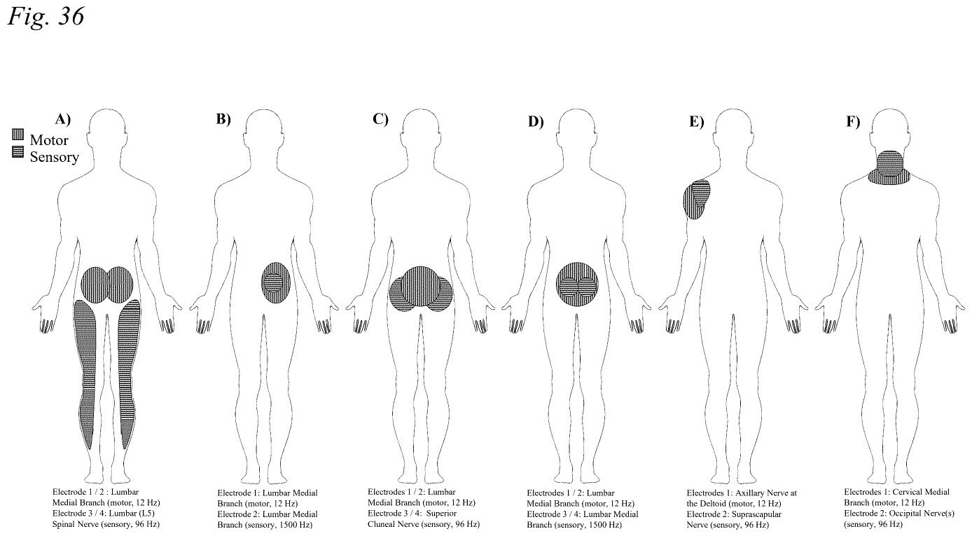

It is appreciated that the system and methods can target one or more than one nerve or nerve branch to apply multimodal stimulation with coordinated motor and sensory stimulation to provide robust stimulation coverage and the relief of pain from multiple etiologies and bodily distributions. FIG. 36 A-F are non-limiting examples coordinated multimodal (e.g., bimodal) stimulation targets to treat various regions of pain throughout the body. As a non-limiting example, as shown in FIGS. 36A-D, the system and methods may apply multimodal (e.g., bimodal) stimulation for the relief of back pain from multiple etiologies and bodily distributions.

FIG. 36A illustrates example regions of pain treated by multimodal (e.g., bimodal) stimulation with two electrodes bilaterally targeting the lumbar medial branch with a lower frequency (e.g., 12 Hz) and two electrodes bilaterally targeting the lumbar spinal nerve with a higher frequency (e.g., 96 Hz). In FIG. 36A, multimodal (e.g., bimodal) stimulation provides pain relief in the back and legs (e.g., bilaterally) by coordinating stimulation of the lumbar medial branch of the dorsal ramus (e.g., via motor stimulation) and the lumbar spinal nerve (e.g., via sensory stimulation).

FIG. 36B illustrates example regions of pain treated by multimodal (e.g., bimodal) stimulation with two electrodes targeting lumbar medial branch, with each electrode providing stimulation at a different frequency (e.g., 12 Hz and 1500 Hz). In FIG. 36B, multimodal (e.g., bimodal) stimulation provides pain relief in the back by coordinating motor and sensory stimulation of the lumbar medial branch of the dorsal ramus.

FIG. 36C illustrates example regions of pain treated by multimodal (e.g., bimodal) stimulation with two electrodes targeting the lumbar medial branch at a lower frequency (e.g., 12 Hz) and two electrodes targeting the superior cluneal nerve at a higher frequency (e.g., 96 Hz). In FIG. 36C, multimodal (e.g., bimodal) stimulation provides pain relief in the back and buttocks by coordinating stimulation of the lumbar medial branch of the dorsal ramus (e.g., via motor stimulation) and the cluneal nerve (e.g., superior cluneal nerve) (e.g., via sensory stimulation).

FIG. 36D illustrates example regions of pain treated by multimodal (e.g., bimodal) stimulation with 2 electrodes targeting the lumbar medial branch at a lower frequency (e.g., 12 Hz) and two electrodes targeting the lumbar spinal nerve at a higher frequency (e.g., 1500 Hz). In FIG. 36D, multimodal (e.g., bimodal) stimulation provides pain relief in the back by coordinating stimulation of the lumbar medial branch of the dorsal ramus (e.g., via motor stimulation at 12 Hz and sensory stimulation at 15000 Hz).

As shown in additional non-limiting examples in FIG. 36E-F, multimodal (e.g., bimodal) stimulation provides pain relief in the shoulder by coordinating stimulation of the axillary nerve at the deltoid (e.g., via motor stimulation) and the suprascapular nerve (e.g., via sensory stimulation) or provides pain relief in the head and neck by coordinating stimulation of the cervical medial branch of the dorsal ramus (e.g., via motor stimulation) and the occipital nerve(s) (e.g., via sensory stimulation).

As non-limiting examples, the electrical stimulation system may provide a multimodal (e.g., bimodal) stimulation via the coordinated delivery of motor stimulation at the axillary nerve at the deltoid and sensory stimulation at the suprascapular nerve for the bimodal treatment of shoulder pain; the electrical stimulation system may provide a multimodal (e.g., bimodal) stimulation via the coordinated delivery of motor stimulation at the cervical medial branch of the dorsal ramus and sensory stimulation at the occipital nerve for the bimodal treatment of head and neck pain; the electrical stimulation system may provide a multimodal (e.g., bimodal) stimulation via the coordinated delivery of motor stimulation at the lumbar medial branch of the dorsal ramus and sensory stimulation at the spinal nerve for the bimodal treatment of back and leg pain; the electrical stimulation system may provide a multimodal (e.g., bimodal) stimulation via the coordinated delivery of motor stimulation at the femoral nerve more distally (e.g., at the vastus medialis oblique) and sensory stimulation at the femoral nerve more proximally (e.g., just distal to the inguinal crease) for the bimodal treatment of knee pain. It is to be appreciated that system can provide other multimodal (e.g., bimodal) stimulation or coordinated motor stimulation and/or sensory stimulation at other combinations of the same or different nerve targets with any combination of frequencies or modes for the treatment of pain.

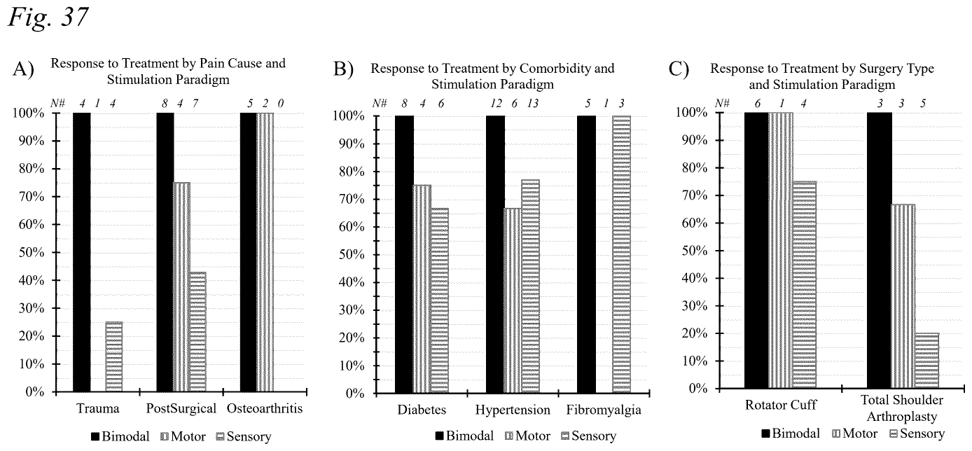

FIG. 37A-C illustrates the responder rate (proportion of patients with greater than equal to 50% pain relief) among patients who received treatment with either unimodal stimulation (unimodal: sensory or unimodal: motor) or bimodal stimulation, presented by comorbidities or conditions known to negatively affect pain treatment response. Patient-reported pain relief at end-of-treatment were collected from a single medical center among patients who underwent peripheral nerve stimulation treatment for shoulder pain (n=57). As shown in FIG. 37A-C, the electrical stimulation system may provide sensory stimulation to the occipital nerve or other peripheral nerve of the head and neck by targeting regions near the greater occipital nerve or third occipital nerve. The same system may also provide motor stimulation to the cervical medial branch nerve at the dorsal ramus concurrently, simultaneously, alternating, in a pattern, overlapping, non-overlapping, randomly, interleaved, and the like. Together, multimodal (e.g., bimodal) stimulation consisting of sensory stimulation and motor stimulation may be used in order to recondition the centrally maintained pain state and alleviate, reduce, or eliminate pain.

As shown in FIG. 20, the electrical stimulation system may provide sensory stimulation to the lumbar spinal nerve by targeting regions near the L4 and L5 lumbosacral trunk. The same system may provide motor stimulation to the lumbar medial branch at the dorsal ramus (for example at L1, L2, L3, L4, or L5) concurrently, simultaneously, alternating, in a pattern, overlapping, non-overlapping, randomly, interleaved, and the like. Together, multimodal (e.g., bimodal) stimulation consisting of sensory stimulation and motor stimulation may be used in order to recondition the centrally maintained pain state and alleviate, reduce, or eliminate pain.

As shown in FIG. 18A, the electrical stimulation system may treat shoulder pain by targeting motor fibers in the axillary nerve. In a non-limiting example, motor stimulation of the axillary nerve via lower frequency(ies) (e.g., 1-20, 2-18, 4-16, 6-16, 8-14, 8-16, 10-16, 10-14, 10-12, 12, 4-20 Hz) with cyclic pulse trains (e.g., having a duty cycle less than 100% such that the stimulation is on for a period of milliseconds, seconds, minutes, or hours, and then off for a period of milliseconds, seconds, minutes, or hours, in repetition, where the duration of the period of time with stimulation being provided (i.e., “on”) may be greater than, less than, or equal to the duration of the period of time with the stimulation not being provided (i.e., “off”), and there may or may not be a period of ramping up and/or down of the intensity, amplitude, pulse width, frequency, or other parameters of stimulation leading into and/or out of the “on” period and/or the “off” period) results in comfortable muscle contraction in the deltoid muscle covering the area of pain.

As shown in FIG. 18B, the electrical system may also treat shoulder pain by targeting sensory fibers in the suprascapular nerve. In a non-limiting example, sensory stimulation of the suprascapular nerve via higher frequency(ies) (e.g., 96 Hz) and continuous pulse trains results in comfortable sensations (e.g., paresthesia) covering the area of pain. The electrical stimulation system may treat shoulder pain by providing multimodal (e.g., bimodal) stimulation, with one lead targeting motor fibers in the axillary nerve via lower frequencies (e.g., 1-20, 2-18, 4-16, 6-16, 8-14, 8-16, 10-16, 10-14, 10-12, 12, 4-20 Hz) and cyclic pulse trains and another lead targeting sensory fibers in the suprascapular nerve via higher frequencies (e.g., 96 Hz) and continuous pulse trains. The coordinated motor stimulation and sensory stimulation mimics the natural coordination between sensory and motor signals from the periphery as they converge in the central nervous system and provides pain relief to systems disrupted by disease, trauma, overuse, or other maladaptive changes. Patient-reported pain relief at end-of-treatment were collected from a single medical center among patients who underwent PNS treatment for shoulder pain (n=57).

As shown in FIG. 18C, the percentage of patients with greater than or equal to 50% pain relief is greater when the electrical stimulation provides coordinated motor and sensory stimulation versus motor stimulation alone and sensory stimulation alone. As shown in FIG. 18D, the average patient-reported pain relief at end of treatment is greater when the electrical stimulation provides coordinated motor and sensory stimulation versus motor stimulation alone and sensory stimulation alone.

FIG. 37A illustrates the proportion of patients with pain from trauma or surgery or osteoarthritis and greater than or equal to 50% pain relief is greater when the electrical stimulation provides coordinated multimodal motor and sensory stimulation (“Bimodal”) versus unimodal motor stimulation alone and unimodal sensory stimulation alone. As shown in FIG. 37A, the proportion of patients with pain from trauma or surgery who experience greater than or equal to 50% pain relief is greater when the electrical stimulation provides coordinated motor and sensory stimulation versus motor stimulation alone and sensory stimulation alone. The proportion of patients with pain from osteoarthritis who experience greater than or equal to 50% pain relief is greater when the electrical stimulation provides coordinated motor and sensory stimulation versus motor stimulation alone and equal when the electrical stimulation provides sensory stimulation alone.

FIG. 37B illustrates that the proportion of patients with diabetes (type I or II) and/or hypertension or fibromyalgia with greater than or equal to 50% pain relief is greater when the electrical stimulation provides coordinated motor and sensory stimulation (“Bimodal”) versus unimodal motor stimulation alone and unimodal sensory stimulation alone. As shown in FIG. 37B, the proportion of patients with diabetes (type I or II) and/or hypertension (comorbidities known to affect negatively pain treatment response) and greater than or equal to 50% pain relief is greater when the electrical stimulation provides coordinated motor and sensory stimulation versus motor stimulation alone and sensory stimulation alone. The proportion of patients with fibromyalgia (another comorbidity known to affect negatively pain treatment response) who experience greater than or equal to 50% pain relief is greater when the electrical stimulation provides coordinated motor and sensory stimulation versus motor stimulation alone and equal when the electrical stimulation provides sensory stimulation alone.

FIG. 37C illustrates that the proportion of patients with post-surgical pain following rotator cuff or shoulder arthroplasty surgery with greater than or equal to 50% pain relief is greater when the electrical stimulation provides coordinated motor and sensory stimulation (“Bimodal”) versus unimodal sensory stimulation alone or unimodal motor stimulation alone. Across each example, multimodal coordinated stimulation (i.e., bimodal stimulation) provided consistently superior outcomes compared to motor or sensory unimodal stimulation in patients with shoulder pain. As shown in FIG. 37C, the proportion of patients with post-surgical pain following rotator cuff or shoulder arthroplasty surgery who experience greater than or equal to 50% pain relief is greater when the electrical stimulation provides coordinated motor and sensory stimulation versus sensory stimulation alone or motor stimulation alone.

Unimodal stimulation (e.g., unimodal motor stimulation, unimodal sensory stimulation, or other unimodal stimulation such as but not limited to unimodal mode X, unimodal mode Y, unimodal mode Z, high frequency, low frequency, sub-threshold, or supra-threshold) may elicit a reduction in pain for individuals with a select number (but not all) of pain etiologies, in patients with comorbidities, and/or in patients after surgery. For example, unimodal motor stimulation and unimodal sensory stimulation or another unimodal stimulation, such as but not limited to unimodal mode X, unimodal mode Y, unimodal mode Z, high frequency, low frequency, sub-threshold, or supra-threshold, may act as a “specific key” to unlock pain relief for a subset of patients, but that “specific key” may fail to provide relief for the majority, all, some, most, or many other subsets of patients, which may include, but are not limited to, patients with disease and/or comorbidities, and/or after surgery. Conversely, by mimicking and/or amplifying the natural coordination of the central nervous system, multimodal (e.g., bimodal) stimulation may act as a “universal key” to unlock pain relief in a far greater number of patients (e.g., most, a plurality, the majority, or all). As a non-limiting example shown in FIG. 37A-C, multimodal (e.g., bimodal) stimulation elicited a larger proportion of responders (e.g., individuals with greater than or equal to 50% pain relief), providing more comprehensive treatment for a greater number of patients with diverse pain etiologies, and/or in patients with comorbidities, and/or in patients after surgery.

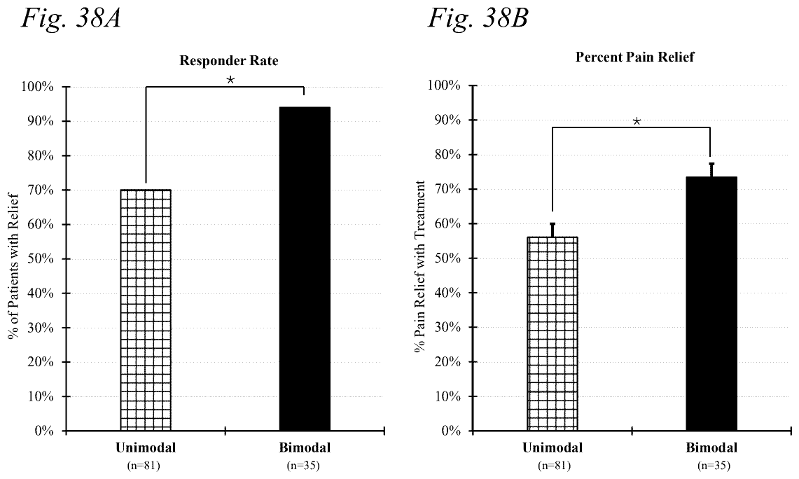

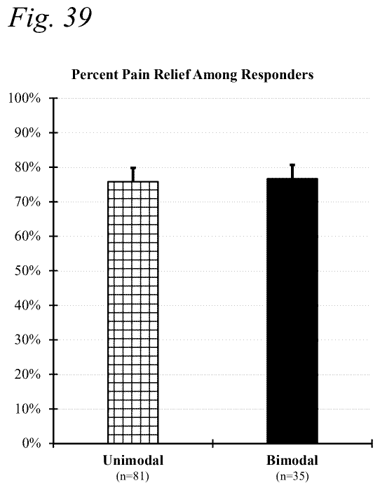

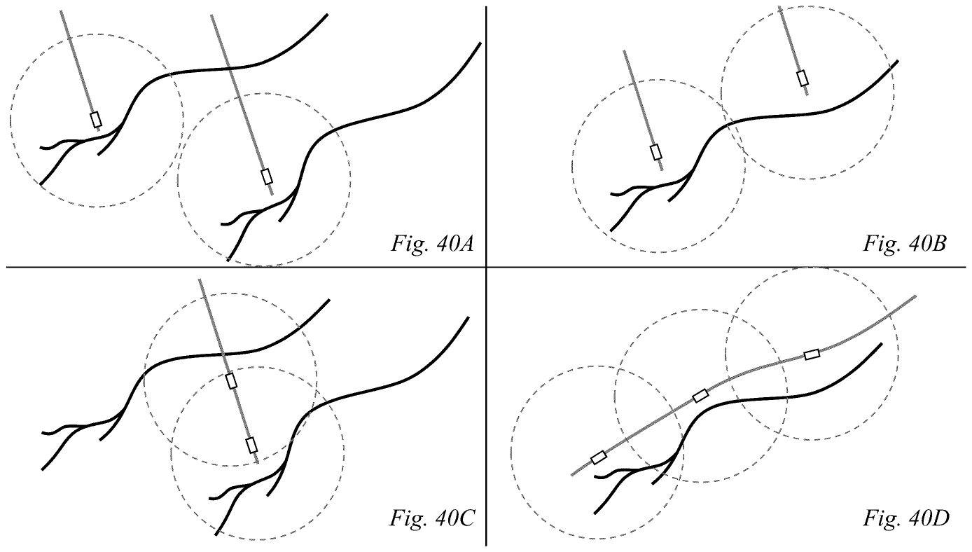

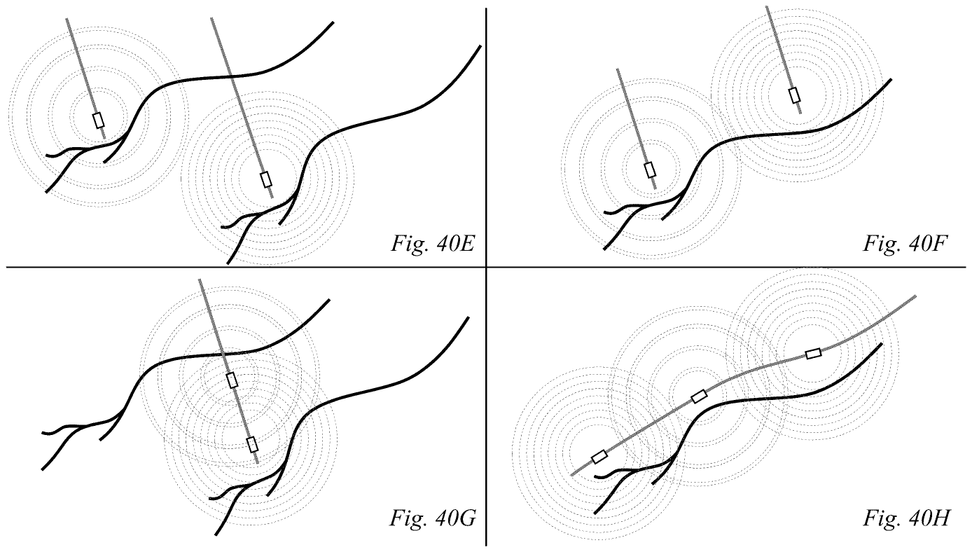

Patient-reported pain relief at end-of-treatment were collected from multiple sites for patients who underwent peripheral nerve stimulation treatment for shoulder pain (n=116). Pain relief were analyzed by stimulation paradigm (unimodal vs. multimodal) using a single-factor ANOVA which, as shown in FIG. 38A, detected a difference in means (p<0.01). The incidence rate of response to treatment (reporting a percent pain relief value of ≥50%) was analyzed using a two-tailed binomial test. As shown in FIG. 38B, a significantly greater proportion of patients receiving multimodal stimulation responded to treatment (94%, 95% Confidence Interval: 86-100%) compared to unimodal stimulation treatment (70%, 95% Confidence Interval: 60-80%). FIG. 39 illustrates pain relief among responders for those who received unimodal stimulation and bimodal stimulation.

For example, FIG. 38A-B represent data collected from multiple medical centers among patients who underwent peripheral nerve stimulation treatment for shoulder pain (n=116). Pain relief was analyzed by stimulation paradigm (unimodal stimulation: unimodal sensory or unimodal-motor; vs. bimodal stimulation) using a single-factor analysis of variance. FIG. 38A illustrates that a treatment of shoulder pain with coordinated bimodal stimulation resulted in a statistically significantly greater percentage of patients who successfully achieved 50% or greater pain relief at the end of a 60-day treatment compared to patients with unimodal stimulation that delivered motor stimulation only or sensory stimulation only (p<0.01). The incidence rate of response to treatment (reporting a percent pain relief value of ≥50%) was analyzed using a two-tailed binomial test. As shown in FIG. 38B, a significantly greater proportion of patients receiving bimodal stimulation responded to treatment (94%; 95% Confidence Interval: 86-100%) compared to unimodal stimulation treatment (70%; 95% Confidence Interval: 60-80%) (p<0.05). Across both analysis endpoints, multimodal coordinated stimulation (i.e., bimodal stimulation) provided statistically superior outcomes compared to unimodal stimulation in patients with shoulder pain.

For example, FIG. 39 illustrates results of an analysis of average patient-reported pain relief among responders (i.e., those reporting a percent pain relief value of ≥50%) at end-of-treatment from collected data from multiple sites for patients who underwent peripheral nerve stimulation with unimodal stimulation (unimodal: sensory or unimodal: motor) (n=81) and bimodal stimulation (n=35) for the treatment of shoulder pain.

The present system and method overcomes multiple limitations of the prior art of peripheral nerve stimulation which required a healthy peripheral nerve to provide pain relief, and it overcomes limitations of stimulation of the central nervous system (e.g., it overcomes limitations of spinal cord stimulation, brain stimulation, deep brain stimulation, etc.) and related stimulation systems (e.g., dorsal root ganglion stimulation) which are unable to produce stimulation-evoked comfortable activation of muscle tissue, muscle contraction, and/or muscle tension that is sufficiently targeted to the local area or region of pain, disease, and/or injury and/or unable to producce stimulation-evoked comfortable activation of peripheral nerve fibers innvervating and sufficiently targeted to and desirably local, focal, focused, robust, and/or relevant to the local area or region of pain, disease, and/or injury to be able to produce changes, including long-term changes in the central nervous system to reverse maladaptive changes associated with chronic pain and/or central sensitization, because the present system and method is able to achieve one or more and/or all of the aforementioned goals and the present system and method is able to achieve these results through peripheral nerve stimulation of a nerve that is not healthy or is no longer healthy, and/or is damaged, injured, cut, partially damaged, severed, partially severed, and/or diseased, and/or contains nerve fibers that are not healthy or are no longer healthy, and/or is damaged, injured, and/or diseased, and/or do not function normally, healthily, and/or in a physiologically typical state.

Patients with diabetes and/or peripheral neuropathy (e.g., caused by diabetes, chemotherapy, etc.) may have nerves that have been damaged in one or more ways (e.g., by chronically high, low, fluctuating, and/or unhealthy blood sugar levels, toxins (e.g., intentional as in chemotherapy or unintentional as in exposure to unintended toxins) and are no longer healthy and/or unable to respond to peripheral nerve stimulation in a way that a healthy nerve would to produce pain relief, but the present system and method overcomes this limitation of the prior art with multimodal (e.g., bimodal) stimulation which may produce (i) a first mode with periodic, cyclical, cycled, intermittent, and/or continuous motor nerve response to elicit a comfortable muscle twitch and/or contraction without unwanted fatigue, which can bypass the unhealthy nerves and/or activate the remaining and/or functioning nerve fibers (which unimodal stimulation would have previously been insufficient to produce the desired pain relief due to not enough healthy nerve fibers, weakened muscle, limited duration per day in which stimulation is tolerated (e.g., no more than 6-12 hours, limited intensity during ramp periods, and no intensity during the off periods), and lack of signal volume or complexity (e.g., biomimetic physiological characteristics) needed for engaging or maximizing analgesic mechanisms (e.g., due to translating stimulation through muscle as a filter)) and (ii) augments this signal and coordinates this signal with a second mode with more pulses per second (higher frequency), approximately an order of magnitude more pulses during the “on” period, more intensity during ramp periods, and continued stimulation during the “off” periods, coordinated in a way that does not fatigue the muscle or receptors or cause pain or discomfort, (e.g., 100% duty cycle), and may provide continuous or almost continuous stimulation for 24 hours per day, ultimately providing multiple orders of magnitude more stimulation than can be provided during unimodal stimulation alone. While in some patients (e.g., diabetic neuropathy patients, patients with chemotherapy-induced neuropathy with unhealthy, diseased, or damaged nerves, or post-surgery patients with damaged nerves, unimodal stimulation (e.g., unimodal motor stimulation, unimodal sensory stimulation, or other unimodal stimulation such as but not limited to unimodal mode X, unimodal mode Y, unimodal mode Z, high frequency, low frequency, sub-threshold, or supra-threshold) is effective (or reliably effective) alone or in alternating fashion (e.g., toggling or flipping from one to the other), the combination of both stimulation modes in a simultaneous, overlapping, or paritally overlapping manner is more effective, robust, and reliable. While unimodal stimulation treatment may be effective in a specific patient type (even if the device could provide both unimodal motor stimulation and unimodal motor stimulation (or other unimodal stimulation) by switching between modes or from providing one mode and then a different mode (e.g., first providing unimodal X stimulation, then switching to provide unimodal Y stimulation, and then back to unimodal X stimulation, and then back to unimodal Y stimulation, etc.), multimodal (e.g., bimodal) stimulation treatment with the present system and method is effective and reliably more effective than systems and methods of unimodal stimulation (e.g., when the present system and method combines two modes of stimulation such that their periods or durations of treatment (or “on” times) are simultaneous in whole or in part or overlapping or paritally overlapping, such as when a first mode is “on” and delivering treatment for 6-12 hours and an additional (e.g., different) mode is “on” and delivering treatment for a period of time that overlaps partially or completely with those 6-12 hours; or when a first mode is “on” and delivering treatment for 6-12 hours and an additional (e.g., different) mode is “on” and delivering treatment for 24 hours such both modes are “on” and delivering treatment for a period of time that overlaps in time for at least 6-12 hours; or when a first mode is “on” and delivering treatment for 6-12 hours and an additional (e.g., different) mode is “on” and delivering treatment for 18-24 hours such both modes are on and delivering treatment for a period of time that overlaps in time. It is appreciated that that patients with unhealthy or damaged nerves may also be treated by another mode (e.g., unimodal mode Z), which also has little, no, minimal, modest, less than optimal, and/or less than desired effect by itself and/or when not applied in a treatment period that overlaps with the treatment period of another mode (e.g., unimodal motor stimulation, unimodal sensory stimulation, or other unimodal stimulation such as but not limited to unimodal mode X, unimodal mode Y, unimodal mode Z, high frequency, low frequency, sub-threshold, or supra-threshold), but when combined in the present system and method as part of the multimodal (e.g., bimodal) treatment such that its treament period overlaps with one or more or both of the other modes, it produces a more reliable and/or desirble effect, pain relief, and/or response.

The present system and method overcomes multiple challenges with combining more than one mode of unimodal (e.g., unimodal motor stimulation, unimodal sensory stimulation, or other unimodal stimulation such as but not limited to unimodal mode X, unimodal mode Y, unimodal mode Z, high frequency, low frequency, sub-threshold, or supra-threshold) stimulation by delivering more than one mode of stimulation simultaneously, partially simultaneously, with partial and/or complete overlapping periods, phases, and/or durations of treatment. For example, the present system and method avoids causing undesirable, unintended, deleterious, and/or other responses or consequences that would or may prevent, limit, and/or detract from combining, providing, and/or delivering more than one mode of stimulation and delivering more than one mode of stimulation simultaneously, partially simultaneously, with partial and/or complete overlapping periods, phases, and/or durations of treatment.

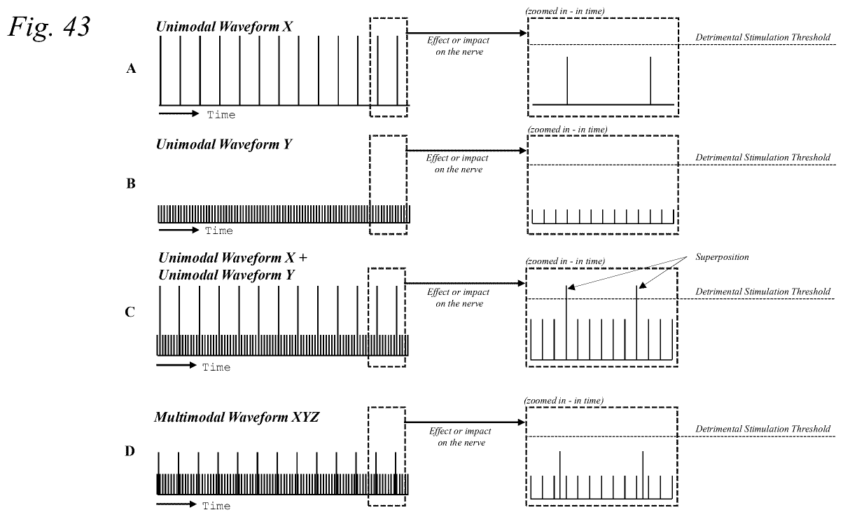

As a non-limiting example, shown in FIG. 43, the present system and method avoids superposition of stimulation modes at the level of individual nerve(s) and/or nerve branch(es) and/or nerve fiber(s) on the scale and time course which could, for example, surpass the detrimimental threshold and/or result in undesireable effects, pain, loss of pain relief, damage and/or injury of or to tissue, damage and/or injury to one or more nerve(s) and/or nerve branch(es) and/or nerve fiber(s), unsafe levels of charge, corrosion and/or damage to the electrode(s) and/or device(s), and avoids one or more of these examples of limiting, undesirable, and/or unintended consequences. For example, FIG. 43A-B illustrate exemplary unimodal stimulation amplitudes and patterns. FIG. 43A illustrates a cyclic stimulation pattern applied at a frequency (e.g., Unimodal Waveform X). FIG. 43B illustrates a continuous stimulation pattern applied at a frequency (e.g., Unimodal Waveform Y). For example, FIG. 43C illustrates a system that adds Unimodal Waveform X and Unimodal Waveform Y, resulting in superposition of stimulation amplitudes and/or intensities and exceeding the “detrimental stimulation threshold” (e.g., tissue damage, increased pain, additional pain, limited analgesic effect, muscle fatigue, nerve fatigue, cramping, and/or other negative outcomes of overstimulation).

FIG. 43D illustrates a non-exhaustive, combinatory stimulation (Multimodal Waveform XYZa) amplitude and/or intensity and/or pattern with two continuous (e.g., tonic) stimulation patterns applied at different frequencies, and these patterns may be applied through the same or more than one electrode. The stimulation amplitude (i.e., height of the pulse illustrated) may be different, such as the pattern applied at a greater frequency (smaller inter-pulse interval, IPI) may be applied at a lesser amplitude than the pattern applied at a lesser frequency (greater IPI). The multimodal stimulation waveform (e.g., Multimodal Waveform XYZ) provides modes of stimulation that desirably do not and/or avoid (or are designed and/or operated to avoid) interact(ion), superposition(ion), influence(ing), and/or interfere(nce) with each other (e.g., in the generation of action potentials one or more nerve(s) and/or nerve fiber(s)) while on the second scale, the modes of stimulation desirably cause a beneficial generation of signals, activation, action potentials, pattern of activation, biomimetic signals, that cause beneficial interaction in the central nervous system and avoid undesirable interaction in the peripheral nervous system (and/or avoid undesirable interaction in the central nervous system as well), essentially enabling the delivery of multiple modes of stimulation in harmony (e.g., which may include the alteration of the modes) in a way such that they are delivered in harmonics and/or in ways that avoid undesirable interference or potential interference with each other near the system(s), device(s), lead(s), electrode(s), peripheral nerve(s) and/or peripheral nerve fiber(s) while causing desirable effects in the central nervous system by peripheral nerve stimulation (operation, performance, delivery, instruction, and/or instruction for use of the disclosed system and method in the periphery and outside, away from, and/or avoiding stimulation (e.g., electrical stimulation of the central nervous system, spinal cord stimulation (SCS), brain stimulation, dorsal root ganglion stimulation (DRGS), and avoiding the dangers, invasiveness, and/or risks of electrical stimulation of the central nervous system, SCS, etc.)).

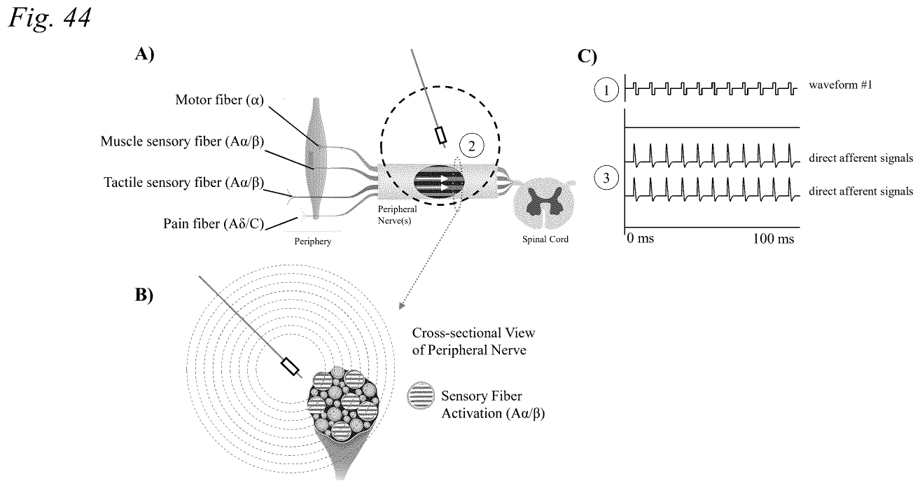

FIG. 44A-C is an illustrative example of peripheral nerve fiber activation following stimulation (e.g., sensory nerve fiber stimulation) for the treatment of pain. Peripheral nerve stimulation approaches may be designed to selectively activate large diameter sensory nerve fibers, where the dotted circles around the electrodes represent stimulation waveforms or the area or zone of activation (e.g., area that is being activated). FIG. 44A illustrates a system with unimodal sensory stimulation that delivers higher frequency stimulation via waveform #1 (e.g., continuous, 100 Hz) to selectively activate large diameter afferent nerves and produce comfortable sensations (e.g., paresthesia) in the region of pain. FIG. 44B represents an illustration of the cross-sectional view of the nerve fibers activated by stimulation. FIG. 44C shows the neural firing signal and signal transduction along the nerves as a result of the application of stimulation and activation of neural fibers within the zone of activation.

As an example, the present system and method can overcome limitations of previous systems and methods via scheduling, timing, timecourse, method, system, location of placement, stimulation parameters, and/or scale of stimulation parameters and/or a combination of these factors, including the alteration of one, two, and/or more of the modes of stimulation such that they maintain their original beneifit and/or amplify or magnify their benefit and effect (e.g., beyond what was possible, produced, or typical individually) while avoiding interference with each other, such that on one or a first scale (e.g., a small, smaller, less, lesser, or micro scale) that is 1, 2, 3, 4, 5, 6, 7, 8, 9, 10, 11, 12 or more times and/or orders of magnitude (e.g., 10\hat{ }1, 10\hat{ }2, 10\hat{ }3, 10\hat{ }4, 10\hat{ }5, 10\hat{ }6, 10\hat{ }7, 10\hat{ }8, 10\hat{ }9, 10\hat{ }10, 10\hat{ }11, 10\hat{ }12 or more) times smaller or less than another, other, or second scale (e.g., a large, larger, greater, or macro scale) that is 1, 2, 3, 4, 5, 6, 7, 8, 9, 10, 11, 12 or more times and/or orders of magnitude (e.g., 10\hat{ }1, 10\hat{ }2, 10\hat{ }3, 10\hat{ }4, 10\hat{ }5, 10\hat{ }6, 10\hat{ }7, 10\hat{ }8, 10\hat{ }9, 10\hat{ }10, 10\hat{ }11, 10\hat{ }12 or more) times larger, bigger, or greater than the first scale, where on the first scale, the modes of stimulation desirably do not and/or avoid (or are designed and/or operated to avoid) interact(ion), superposition(ion), influence(ing), and/or interfere(nce) with each other (e.g., in the generation of action potentials one or more nerve(s) and/or nerve fiber(s)) while on the second scale, the modes of stimulation desirably cause a beneficial generation of signals, activation, action potentials, pattern of activation, biomimetic signals, that cause beneficial interaction in the central nervous system and avoid undesirable interaction in the peripheral nervous system (and/or avoid undesirable interaction in the central nervous system as well), essentially enabling the delivery of multiple modes of stimulation in harmony (e.g., which may include the alteration of the modes) in a way such that they are delivered in harmonics and/or in ways that avoid undesirable interference or potential interference with each other near the system(s), device(s), lead(s), electrode(s), peripheral nerve(s) and/or peripheral nerve fiber(s) while causing desirable effects in the central nervous system by peripheral nerve stimulation (operation, performance, delivery, instruction, and/or instruction for use of the present system and method in the periphery and outside, away from, and/or avoiding stimulation (e.g., electrical stimulation of the central nervous system, spinal cord stimulation (SCS), brain stimulation, and avoiding the dangers, invasiveness, and/or risks of electrical stimulation of the central nervous system, SCS, etc.)). The present system and method enables and/or may require in some non-limiting examples modification of one or more modes of stimulation such that it can be combined with another mode of stimulation but in a way that preserves and/or enhances its effect and avoids losing the benefit of that or those mode(s) of stimulation and avoids undesirable interference between the modes one one level, scale, and/or location (e.g., in the periphery, at the level of the electrode(s) and peripheral nerve(s) or nerve fiber(s), and/or on a micro scale (e.g., microseconds, milliseconds, and/or other time scales less and/or a fraction of a microsecond, millisecond, and/or 1 second) while causing desirable interaction between the signals that are generated in the nerve(s) and/or nerve fiber(s).

The electrical stimulation system may include at least one, one or more, a plurality of, or at least two different stimulation frequencies. The different stimulation frequencies may be provided through different stimulation electrodes, such as the multiple electrodes 14 shown on lead 12 in FIGS. 23B and 24B. In an embodiment, a first stimulating electrode may provide a first frequency stimulation (e.g., 0.1-20 Hz, 20-100 Hz, 100-500 Hz, 500-15000 Hz) and a second stimulating electrode may provide a second frequency stimulation (e.g., 0.1-20 Hz, 20-100 Hz, 100-500 Hz, 500-15000 Hz), where the first and second frequency stimulations are different. In an embodiment, a first stimulating electrode may provide a first frequency stimulation and a second stimulating electrode may provide a second frequency stimulation, where the first frequency stimulation is higher than the second frequency stimulations. In an embodiment, a first stimulating electrode may provide a first frequency stimulation and a second stimulating electrode may provide a second frequency stimulation, where the first frequency stimulation is lower than the second frequency stimulations.

It is noted that systems with more than two electrodes may include at least two different stimulation frequencies at two different electrodes. In an embodiment, some of the electrodes may provide stimulation at the same frequencies (e.g., two of three electrodes providing the same stimulation frequency and the other one of the electrodes providing a different stimulation frequency; or two of four electrodes each providing a first stimulation frequency and the other two electrodes each providing a second same frequency, where the first and second stimulation frequencies are different, but more than one electrode provides each of the first and/or second stimulation frequencies). In an embodiment, all of the electrodes may provide different stimulation frequencies from each other (e.g., all three electrodes each providing different stimulation frequencies for three total different mixed frequencies; or all four electrodes each providing different stimulation frequencies for four total different mixed frequencies). It is appreciated that the number of electrodes and number of mixed frequencies is not bound.

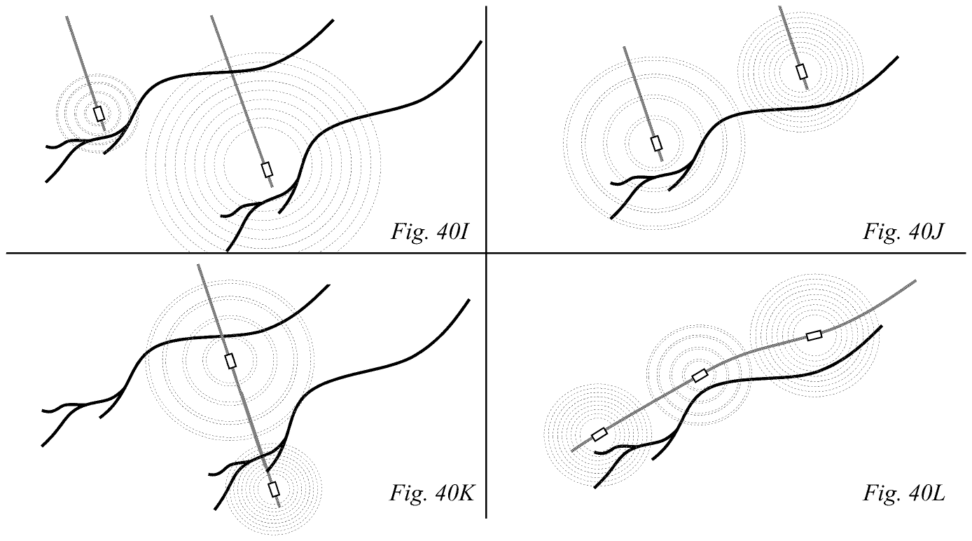

It is noted that systems with one lead may provide stimulation through one electrode, more than one electrode, two electrodes, more than two electrodes, three electrodes, and more than three electrodes. It is noted that systems with two leads may provide stimulation through one electrode, or more than one electrode on each lead. It is appreciated that a system may have n number of leads, each with n number of electrodes. It is appreciated that the number of leads does not have to equal the number of electrodes and the number of electrodes does not have to equal the number of leads. FIG. 40A-L illustrates non-exhaustive options for multimodal (e.g., bimodal stimulation and methods for applying multimodal stimulation to one or more nerves, e.g., peripheral nerves, via one or more leads with one or more electrodes, where the dotted circles around the electrodes represent stimulation waveforms (e.g., cyclic, patterned, or continuous waveforms) or the area or zone of activation (e.g., area that is being activated) that may or may not overlap with other areas or zones. The methods (see FIG. 40I-L, for example) may avoid one or more types of interference from stimulation for one or more electrodes.

FIG. 40A illustrates multimodal stimulation with one system and two leads, with one lead and one electrode providing motor stimulation to one peripheral nerve and/or nerve branch, and another lead and electrode providing sensory stimulation to another nerve and/or nerve branch. In FIG. 40A, a system with two leads, each lead with one electrode, is targeting two separate nerves or nerve branches. FIG. 40B illustrates multimodal stimulation with one system and two leads, with one lead and one electrode providing motor stimulation to one nerve and/or nerve branch, and another lead and electrode providing sensory stimulation to the same nerve and/or nerve branch. In FIG. 40B, a system with two leads, each lead with one electrode, is targeting the nerve or nerve branch. FIG. 40C illustrates multimodal stimulation with one system and one leads, with one electrode providing motor stimulation to one nerve and/or nerve branch, and another electrode providing sensory stimulation to a different nerve and/or nerve branch. In FIG. 40C, a system with one lead with two electrodes is targeting two nerves or nerve branches. FIG. 40D illustrates multimodal stimulation with one system and one lead with three electrodes, with one electrode providing sensory stimulation to one nerve and/or nerve branch, another electrode providing motor stimulation to the same nerve and/or nerve branch at the same or different location, and another electrode providing sensory stimulation to the same nerve and/or nerve branch at the same or different location. In FIG. 40D, a system with one lead with three electrodes is targeting one nerve or nerve branch.

FIG. 40E illustrates multimodal stimulation with one system and two leads, with one lead and one electrode providing motor stimulation (e.g., cyclic waveform) to one nerve and/or nerve branch, and another lead and electrode providing sensory stimulation (e.g., continuous waveform) to another nerve and/or nerve branch. FIG. 40F illustrates multimodal stimulation with one system and two leads, with one lead and one electrode providing motor stimulation (e.g., cyclic waveform) to one nerve and/or nerve branch, and another lead and electrode providing sensory stimulation (e.g., continuous waveform) to the same nerve and/or nerve branch. FIG. 40G illustrates multimodal stimulation with one system and one leads, with one electrode providing motor stimulation (e.g., cyclic waveform) to one nerve and/or nerve branch, and another electrode providing sensory stimulation (e.g., continuous waveform) to a different nerve and/or nerve branch. FIG. 40H illustrates multimodal stimulation with one system and one lead with three electrodes, with one electrode providing sensory stimulation (e.g., continuous waveform) to one nerve and/or nerve branch, another electrode providing motor stimulation (e.g., cyclic waveform) to the same nerve and/or nerve branch at the same or different location, and another electrode providing sensory stimulation (e.g., continuous waveform) to the same nerve and/or nerve branch at the same or different location.

FIG. 40I illustrates multimodal stimulation with one system and two leads, with one lead and one electrode providing motor stimulation (e.g., cyclic waveform) to one nerve and/or nerve branch, and another lead and electrode providing sensory stimulation (e.g., continuous waveform) to another nerve and/or nerve branch. FIG. 40J illustrates multimodal stimulation with one system and two leads, with one lead and one electrode providing motor stimulation (e.g., cyclic waveform) to one nerve and/or nerve branch, and another lead and electrode providing sensory stimulation (e.g., continuous waveform) to the same nerve and/or nerve branch. FIG. 40K illustrates multimodal stimulation with one system and one leads, with one electrode providing motor stimulation (e.g., cyclic waveform) to one nerve and/or nerve branch, and another electrode providing sensory stimulation (e.g., continuous waveform) to a different nerve and/or nerve branch. FIG. 40L illustrates multimodal stimulation with one system and one lead with three electrodes, with one electrode providing sensory stimulation (e.g., continuous waveform) to one nerve and/or nerve branch, another electrode providing motor stimulation (e.g., cyclic waveform) to the same nerve and/or nerve branch at the same or different location, and another electrode providing sensory stimulation (e.g., continuous waveform) to the same nerve and/or nerve branch at the same or different location.

The electrical stimulation system may include one or more lower frequencies (e.g., 1-20, 2-18, 4-16, 6-16, 8-14, 8-16, 10-16, 10-14, 10-12, 12, 4-20 Hz) and at least one highest frequency (e.g., 20-35, 35-45, 45-60, 60-70, 70-85, 85-120, 20-120, 60-120, 96, 120, 120-200, 120-500, 200-500, 500-1500, 500-5000, 5000-15000, 20-15000 Hz). In an embodiment, the one or more lower frequencies (e.g., 1-20, 2-18, 4-16, 6-16, 8-14, 8-16, 10-16, 10-14, 10-12, 12, 4-20 Hz) are subharmonic frequencies of the at least one higher frequency (e.g., 96 Hz). In an embodiment, the one or more lower frequencies are subharmonic frequencies of the at least one highest frequency divided by an integer. In an embodiment, subharmonic frequencies may include frequencies below the fundamental frequency of an oscillator in a ratio of 1/n, where n is a positive integer. In another embodiment, one or more lower frequencies (e.g., 1-20, 2-18, 4-16, 6-16, 8-14, 8-16, 10-16, 10-14, 10-12, 12, 4-20 Hz) are not subharmonic frequencies of one highest frequency (e.g., 20-35, 35-45, 45-60, 60-70, 70-85, 85-120, 20-120, 60-120, 96, 120, 120-200, 120-500, 200-500, 500-1500, 500-5000, 5000-15000, 20-15000 Hz).

In an embodiment, the electrical stimulation system and methods may include timing for the start and stop of stimulus pulses that is determined by: (1) Assuming that all electrodes were stimulated at the same frequency. In an embodiment, the N channels (electrodes) may have their stimulus pulses interleaved such that there is roughly equal time between each successive pulse with a sequence of all N channels repeating at the stimulus frequency. Each of these N stimulus pulses may be in its own stimulus timing slot a specified delay after a first channel with a highest frequency stimulus. (2) All channels (leads) at the highest stimulus frequency may occur in each of their time slots. (3) A subharmonic channel (lead) may generate a stimulus pulse in every Mth of its time slots where M=subharmonic ratio; i.e., the ratio of the fastest stimulus frequency to the stimulus frequency on the subharmonic channel (lead) in question. As a non-limiting illustrative example, if one channel (channel 1) delivers stimulation at 96 Hz and a second channel (channel 2) delivers stimulation at 12 Hz (a subharmonic frequency of 96 Hz in a ratio of ⅛), and the channels deliver stimulation pulses in stimulus timing slots with specified delay between the two channels, the stimulation pulses from channel 1 may be delivered in each time slot to produce a stimulation frequency of 96 Hz while the stimulation pulses from channel 2 may be delivered in every 8^{th} time slot to produce a stimulation frequency of 12 Hz.

In an embodiment, the only timing hardware or software needed for determining or providing the determined electrical stimulus may be the same or a slight modification of that required for the generation of regular interleaved stimulus pulses all at the same frequency. For example, if the there are N stimulating electrodes or leads, a clock operating at N times the largest stimulus frequency could be directed to the first electrode or lead, then the second electrode or lead and so on until one reaches the Nth electrode or lead. This would allow all N stimulus pulses to be evenly spaced and operating at the same frequency (e.g., at any stimulation frequency necessary to provide pain relief). If one or more of the stimulation electrodes or leads only stimulated on every Mth clock tick direct to them, then that stimulation electrode or lead would be stimulating at a subharmonic frequency (e.g., in one embodiment, 12 Hz could be used as a subharmonic frequency of 96 Hz). In the extreme case, the N stimulation electrode or lead could each have a different frequency.

The different stimulation frequencies may be provided concurrently, simultaneously, alternating, in a pattern, overlapping, non-overlapping, randomly, interleaved, and the like. In an embodiment, one system containing two or more electrodes with two or more frequencies may use one or more electrode independently. In an embodiment, one system containing two or more electrodes with two or more frequency ranges may use more than one electrode simultaneously. In one embodiment, the different stimulation frequencies are applied simultaneously, where the lower frequency channel's subharmonic pulses occur at the same time as pulses in the higher frequency channel. Alternatively, these pulses could be generated such that they are arranged in an interleaved fashion, where the pulse presentation varies (e.g., not overlapping, randomly overlapping, pattern of overlap) and/or changes over time (e.g., where the pulses presentation overlap changes over time). In an embodiment, harmonic frequencies for one, more than one, multiple, many channels or electrodes or leads may adjust over time in coordination with dynamic or static changes to parameters (e.g., frequencies) of one or more channels or electrodes.

In an embodiment, one stimulation frequency targets a motor nerve response (e.g., motor stimulation) resulting in cyclical muscle twitches and/or muscle contractions while another stimulation frequency targets a sensory nerve response (e.g., sensory stimulation) resulting in a comfortable paresthesia. In an embodiment, both motor and sensory nerve responses overlap to mimic signals in the central nervous system. Alternatively, motor and sensory nerve responses could be randomly overlapping or overlapping in a distinct pattern. The described system (e.g., a coordinated system, or a coordinated bimodal system, or a bimodal system) targets both sensory nerve responses to provide a continuous paresthesia over the region of pain and periodic motor nerve response to provide a comfortable muscle twitch and/or contraction without unwanted fatigue. The electrical stimulation system may provide stimulation to an innervated muscle at a lower frequency (e.g., 1-20, 2-18, 4-16, 6-16, 8-14, 8-16, 10-16, 10-14, 10-12, 12, 4-20 Hz) with or without a modulating envelope (e.g., a pattern of intensity modulation or variation that is applied to the continuous pulse train) while stimulating nerve branches at a higher frequency (e.g., 120, 96, 60-70, 70-85, 85-100, 100-120, 60-120 Hz) with or without a modulating envelope. It is noted that the opposite is also herein contemplated and disclosed where the electrical stimulation system may provide stimulation to an innervated muscle at a higher frequency (e.g., 120, 96, 60-70, 70-85, 85-100, 100-120, 60-120 Hz) with or without a modulating envelope while stimulating nerve branches at a lower frequency (e.g., 1-20, 2-18, 4-16, 6-16, 8-14, 8-16, 10-16, 10-14, 10-12, 12 4-20 Hz) with or without a modulating envelope. In an embodiment, the system and method may target afferent nerve fiber types and/or efferent nerve fiber types that may differ from sensory and motor fibers, such as, but not limited to, sympathetic nerves and/or parasympathic nerves.

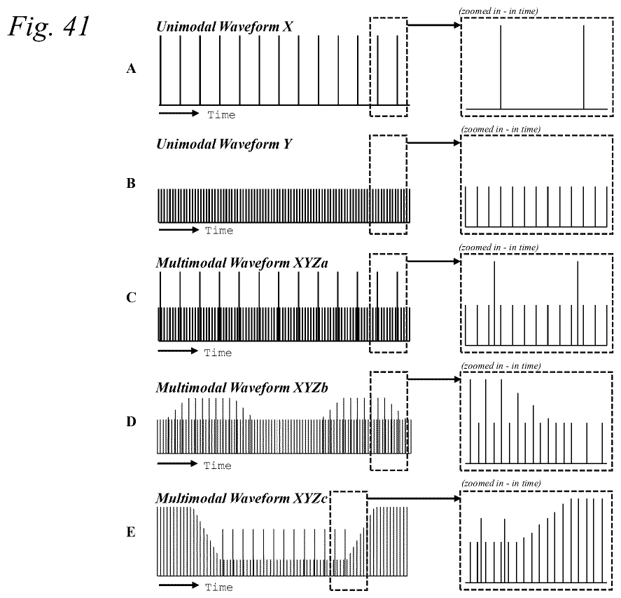

FIG. 41A-B illustrate exemplary unimodal stimulation amplitudes and stimulation pulse patterns. FIG. 41A illustrates a unimodal cyclic stimulation pattern applied at a frequency (e.g., Unimodal Waveform X). FIG. 41B illustrates a continuous stimulation pattern applied at a frequency (e.g., Unimodal Waveform Y).

FIG. 41C-E illustrate non-exhaustive, combinatory stimulation amplitudes and stimulation pulse patterns that can be applied to one or more electrodes. FIG. 41C illustrates a coordinated multimodal waveform (e.g., Multimodal Waveform XYZa) with two continuous (e.g., tonic) stimulation patterns applied at different frequencies, and these patterns may be applied through the same or more than one electrode. The stimulation amplitude (i.e., height of the pulse illustrated) may be different, such as the pattern applied at a greater frequency (smaller inter-pulse interval, IPI) may be applied at a lesser amplitude than the pattern applied at a lesser frequency (greater IPI). In an embodiment, as illustrated in FIG. 41C, a system and method can provide stimulation with intensities that are different between leads, electrodes, and/or channels.

As an example, a system and method can dynamically modulate the amplitude (e.g., 0.1-20mA, or 0.05-40 mA, or 0.01-200 mA) on each individual channel, can dynamically modulate intensities (e.g., pulse duration, amplitude, or a combination of pulse duration and amplitude, or a combination of any parameter or group of parameters) on each individual channel, can dynamically modulate the pulse duration (e.g., 1-300 microseconds, or 5-1000 microseconds, or 1-10,000 microseconds) on each individual channel, and can cycle each individual channel or electrode on (e.g., above zero intensity) or off (e.g., zero intensity), as illustrated in FIG. 41D. FIG. 41D illustrates a coordinated multimodal waveform (e.g., Multimodal Waveform XYZb) with one continuous (e.g., tonic) stimulation pattern and a phasic (e.g., duty cycle<100%) stimulation pattern applied within an envelope, and these stimulation patterns may be applied through the same or more than one electrode. The stimulation amplitude may be unchanged for one pattern (e.g., for the continuous stimulation pattern) or applied within an envelope (e.g., for the phasic stimulation pattern). The stimulation patterns may be applied at the same or different frequencies. As a non-limiting example, the stimulation pattern applied using a continuous pattern may be applied at a greater frequency than the stimulation applied using a phasic pattern.

In an embodiment, one mode may be modulated to increase the effect or noticeability of another mode, as illustrated in FIG. 41E. FIG. 41E illustrates a coordinated multimodal waveform (e.g., Multimodal Waveform XYZa), with a non-exhaustive combination of two phasic patterns (e.g., duty cycle<100%), and these stimulation patterns may be applied through the same or more than one electrode. The stimulation patterns may differ in stimulation frequency and amplitude. The stimulation patterns may use a constant amplitude or include “ramping” amplitudes whereby the amplitude changes at a constant rate until meeting a pre-programmed value. As a non-limiting example, a system and method may identify one electrode providing sensory stimulation (e.g., at 100 Hz) and decrease intensity (e.g., amplitude, pulse duration) to amplify another electrode providing motor stimulation (e.g., at 12 hz) and avoid overaction and/or muscle fatigue. It is appreciated that a system and method may modulate any, one, a combination, or all parameters (e.g., frequency, pulse duration, amplitude, duty cycle, pattern of stimulus pulses, polarity, a predetermined number of phases, and waveform shape) for any number of leads, electrodes, and/or channels connected to the system.

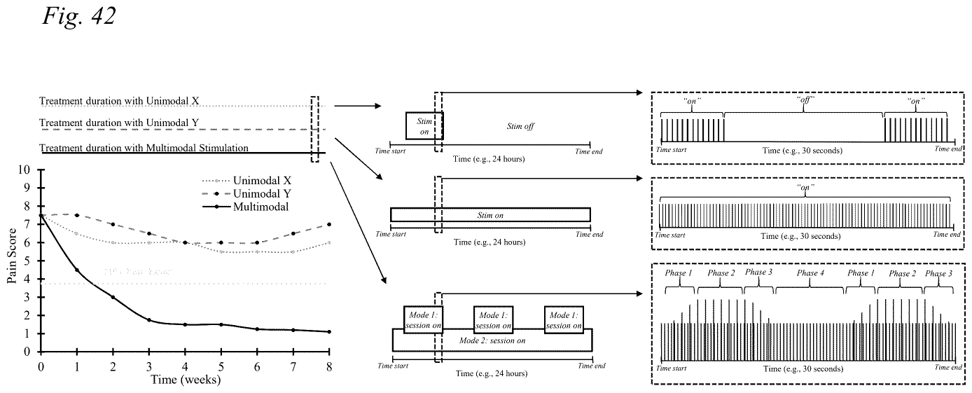

As a non-limiting example, as shown in FIG. 42, a temporary treatment with Unimodal X Stimulation (e.g., motor stimulation, or sensory stimulation, or single mode stimulation, or single frequency stimulation), with stimulation being delivered for a portion, some, or part of the day (e.g., 1 hour, 6-12 hours, 1-6 hours, 12 hours, 10 hours, 12-18 hours, or 18-23 hours), with stimulation pulses being delivered cyclically at a frequency (e.g., with periods of stimulation “on” followed by periods of stimulation “off”, followed by periods of stimulation “on”, and so on so forth while the stimulation is being delivered) or Unimodal Y Stimulation (e.g., motor stimulation, or sensory stimulation, or single mode stimulation, or single frequency stimulation), with stimulation being delivered continuously for most, the majority, or all of the day, with stimulation pulses being delivered continuously at a frequency is less effective, produces less pain relief, has fewer people experiencing relief, and/or treats fewer patients than compared to multimodal (e.g., bimodal) stimulation, with a coordinated stimulation signal that is provided throughout the day (e.g., as needed), with the coordinated signal being made up of (i) one mode (e.g., mode 1) deliving cyclical pulses at a frequency with an intensity that has multiple phases, such there is a period where intensity is increasing (e.g., “ramp up”, Phase 1), a period where intensity plateaus (e.g., Phase 2), a period where intensity is decreasing (e.g., “ramp down”, Phase 3), and a period where the intensity of mode 1 is off (e.g., Phase 4), and (2) one mode (e.g., mode 2) delivering continuous pulses at the same or a different frequency with an intensity that is modulated as needed (e.g., to increase or decrease the effectiveness or result of mode 1).

FIG. 42 illustrates exemplary outcomes after a temporary treatment from systems providing unimodal X stimulation or unimodal X stimulation versus multimodal (e.g., bimodal) stimulation. It is appreciated that unimodal X or unimodal Y stimulation may correspond to sensory stimulation, motor stimulation, stimulation at lower frequencies (e.g., 4-20 Hz), stimulation at higher frequencies (e.g., 20-60 Hz, 60-120 Hz, 120-500 Hz, 500-15000 Hz, 1000-20000 Hz), sub-threshold stimulation, burst stimulation, or other stimulation patterns or waveforms. Here, unimodal X stimulation is shown as a cyclic waveform with periods of intensity “on” and periods without intensity or with zero intensity “off”. Unimodal Y stimulation is shown as a continuous waveform (e.g., only with periods of intensity “on”). As a non-limiting example, multimodal stimulation (e.g., a coordinated stimulation which simultaneously concurrently, interleaved, and/or in another pattern provides signals to mimic the natural coordination between sensory and motor signals in the central nervous system) is shown with one mode having a continuous waveform (e.g., only with periods of intensity “on”) and another mode with increasing intensity during a “ramp up” (e.g., Phase 1), constant or consistent intensity during a plateau region (e.g., Phase 2), decreasing intensity during a “ramp down” (e.g., Phase 3), and no intensity (e.g., Phase 4). As an example, multimodal (e.g., bimodal) stimulation results in faster relief (e.g., avoiding a delayed response, decreasing time to response, increasing number of early responders, avoiding issues with compliance) and more substantial relief (e.g., greater) compared to unimodal X and Y stimulation.

The described electrical stimulation systems and methods can overcome limitations of unimodal stimulation, wherein time-invariant stimulations lack the complexity needed or the termporal patterning needed for engaging or maximizing analgesic mechanisms and lead to desensitization of sensations and/or the gradual reduction in analgesic efficacy. The described electrical stimulation systems and methods can overcome the limitations of unimodal stimulation, wherein lower frequencies alone may lack robustness or volume of input by relying on indirect proprioceptive pathways such that therapeutic mechanisms are not maximized. The described electrical stimulation systems and methods can overcome limitations of unimodal stimulation, wherein stimulation can lead to incomplete coverage of pain, incomplete relief, minimal relief, no relief, and/or waning of relief over time. The described electrical stimulation systems and methods can overcome limitations of unimodal stimulation, wherein the effectiveness of therapy is dependent in part on the health of the nerve, and is reduced in patients with unhealthy nerves and/or nerve damage or pre-existing nerve damage (natural or iatrogenic and/or caused by trauma, chemicals, surgery, diseases, and/or disease progression) such as diabetes (type I and/or II), neuropathy, chemotherapy induced neuropathy, obesity, hypertension, and/or other diseases or disorders. The described electrical stimulation systems and methods can prevent, minimize, or reduce inconsistent and potentially problematic electrochemistry changes at the electrode(s) and inconsistent and potentially problematic changes at the anode (return electrode) with stimulus pulses on different stimulation leads being delivered at the same time or with only a short delay between pulses.

Although the embodiments of the present system and method have been illustrated in the accompanying drawings and described in the foregoing detailed description, it is to be understood that the present system and method is not to be limited to just the embodiments disclosed, but that the system and method described herein is capable of numerous rearrangements, modifications and substitutions without departing from the scope of the claims hereafter. The claims as follows are intended to include all modifications and alterations insofar as they come within the scope of the claims or the equivalent thereof.

1. A multimodal peripheral nerve stimulation system comprising:
a first lead configured for insertion into a body proximal to one or more regions of pain, wherein the first lead comprises one or more first electrodes integrally formed with the first lead and wherein the one or more first electrodes are configured to be positioned near at least one nerve or nerve branch that innervates the one or more regions of pain;
a second lead configured for insertion into a body proximal to a second one or more regions of pain, wherein the second lead comprises one or more second electrodes integrally formed with the second lead and wherein the one or more second electrodes are configured to be positioned near at least one nerve or nerve branch that innervates the second one or more regions of pain; and
an electrical stimulation device operatively coupled to the first and second leads, the electrical stimulation device configured to apply electrical stimulation comprising a first mode of stimulation comprising a first set of stimulation parameters through the first lead during a first treatment period, and the electrical stimulation comprises a second mode of stimulation comprising a second set of stimulation parameters through the second lead during a second treatment period, wherein the first and second treatment periods overlap in time and wherein the first and second set of stimulation parameters are different and produce different effects on different target neural fibers to activate multiple mechanisms of pain relief that convergently modulate central nervous system plasticity associated with the one or more regions of pain and the second one or more regions of pain.

2. The multimodal peripheral nerve stimulation system of claim 1, wherein the first set of stimulation parameters comprises a first parameter of frequency.

3. The multimodal peripheral nerve stimulation system of claim 2, wherein the first set of stimulation parameters comprises a second parameter of stimulation selected from a group consisting of: frequency, pulse duration, amplitude, duty cycle, pattern of stimulus pulses, polarity, a predetermined number of phases, and waveform shape.

4. The multimodal peripheral nerve stimulation system of claim 3, wherein the second set of stimulation parameters comprises a third parameter selected from a group consisting of: frequency, pulse duration, amplitude, duty cycle, pattern of stimulus pulses, polarity, a predetermined number of phases, and waveform shape.

5. The multimodal peripheral nerve stimulation system of claim 4, wherein the first parameter of frequency is different from the frequency of the second parameter of stimulation.

6. The multimodal peripheral nerve stimulation system of claim 5, wherein the first parameter of the frequency is provided by the one or more first electrodes and wherein the first parameter of the frequency is a subharmonic frequency of a stimulation frequency provided by the one or more second electrodes, divided by an integer.

7. The multimodal peripheral nerve stimulation system of claim 6, wherein the subharmonic frequency is continuously provided by the one or more first electrodes.

8. The multimodal peripheral nerve stimulation system of claim 6, wherein the subharmonic frequency is provided at varying burst lengths by the one or more first electrodes.

9. The multimodal peripheral nerve stimulation system of claim 1, wherein the one or more first electrodes provide stimulation at a different frequency from the one or more second electrodes.

10. The multimodal peripheral nerve stimulation system of claim 1, wherein the one or more first electrodes provide stimulation at an equivalent frequency from the one or more second electrodes.

11. The multimodal peripheral nerve stimulation system of claim 1, wherein the one or more electrodes comprises at least three first electrodes wherein two or more of the at least three first electrodes provide stimulation at different frequencies and one or more of the at least three first electrodes provide stimulation at an equivalent frequency from any of the frequencies of the two or more of the at least three first electrodes.

12. The multimodal peripheral nerve stimulation system of claim 1, wherein the one or more electrodes comprises at least three first electrodes wherein two or more of the at least three first electrodes provide stimulation at different frequencies and one or more of the at least three first electrodes provide stimulation at a different frequency from any of the frequencies of the two or more of the at least three first electrodes.

13. The multimodal peripheral nerve stimulation system of claim 1, wherein modulation of stimulation frequency provided by the one or more electrodes avoids overactivation or fatigue of one or both of the nerve and muscle.

14. The multimodal peripheral nerve stimulation system of claim 1, wherein a neurostimulation pulse of the electrical stimulation delivered from the one or more electrodes is biphasic or multiphasic, and/or symmetrical or asymmetrical, and/or is rectangular, exponential, or sinusoidal, or is a combination of rectangular, exponential, and/or sinusoidal.

15. The multimodal peripheral nerve stimulation system of claim 1, wherein a neurostimulation pulse of the electrical stimulation delivered from the one or more electrodes comprise continuous or intermittent trains.

16. The multimodal peripheral nerve stimulation system of claim 1, wherein a neurostimulation pulse of the electrical stimulation delivered from the one or more electrodes comprises duty cycles that are regular and repeatable from one intermittent burst to another.

17. The multimodal peripheral nerve stimulation system of claim 1, a neurostimulation pulse of the electrical stimulation delivered from the one or more electrodes comprises duty cycles with each burst varying in a pre-programmed manner.

18. The multimodal peripheral nerve stimulation system of claim 1, wherein a neurostimulation pulse of the electrical stimulation delivered from the one or more electrodes comprises duty cycles with each burst varying in a non-repeating manner.

19. The multimodal peripheral nerve stimulation system of claim 1, wherein a neurostimulation pulse of the electrical stimulation delivered from the one or more electrodes comprises duty cycles with each burst varying in a random or pseudo random manner.

20. The multimodal peripheral nerve stimulation system of claim 1, wherein a neurostimulation pulse of the electrical stimulation delivered from the one or more electrodes comprises duty cycles with each burst varying in a combination of repeatable, random or pseudo random patterns, and/or non-repeating preprogrammed patterns.

21. The multimodal peripheral nerve stimulation system of claim 1, wherein a first neurostimulation pulse of the electrical stimulation delivered from the one or more electrodes comprises avoids overlapping with a second neurostimulation pulse of the electrical stimulation delivered from the second one or more electrodes.

22. The multimodal peripheral nerve stimulation system of claim 21, wherein avoidance of overlapping of the first and second neurostimulation pulse avoids undesirable effects of superposition while maintaining coordinated delivery of multiple modes.

23. The multimodal peripheral nerve stimulation system of claim 21, wherein avoidance of overlapping of the first and second neurostimulation pulse avoids undesirable effects of muscle fatigue while maintaining coordinated delivery of multiple modes.

24. The multimodal peripheral nerve stimulation system of claim 21, wherein avoidance of overlapping of the first and second neurostimulation pulse avoids undesirable effects of nerve fatigue while maintaining coordinated delivery of multiple modes.

25. The multimodal peripheral nerve stimulation system of claim 21, wherein avoidance of overlapping of the first and second neurostimulation pulse avoids undesirable effects of tissue damage while maintaining coordinated delivery of multiple modes.

26. The multimodal peripheral nerve stimulation system of claim 1, wherein the one or more first electrodes deliver stimulation intended to activate large diameter primary afferent fibers.

27. The multimodal peripheral nerve stimulation system of claim 1 wherein the one or more second electrodes deliver stimulation intended to activate large diameter efferent motor fibers.

28. The multimodal peripheral nerve stimulation system of claim 1, wherein the one or more second electrodes deliver stimulation intended to generate indirect proprioceptive afferent signals.

29. (canceled)

30. (canceled)

31. (canceled)

32. (canceled)

33. (canceled)

34. (canceled)

35. (canceled)

36. (canceled)

37. (canceled)

38. (canceled)

39. A method comprising:
placing a lead comprising a plurality of electrodes integrally formed with the lead into a portion of a body that is proximal to a region of pain;
positioning the plurality of electrodes near at least one nerve or nerve branch that innervates the region of pain; and
electrically stimulating the nerve, wherein the electrical stimulation comprises a first set of stimulation parameters applied through a first electrode of the plurality of electrodes and a second set of stimulation parameters applied through a second electrode of the plurality of electrodes, wherein the first and second sets of stimulation parameters produce a synergistic effect to provide pain relief to the region of pain.

40. The method of claim 39, wherein the electrical stimulation reconditions a centrally maintained pain state.

41. The method of claim 39, wherein the first set of stimulation parameters comprises a first frequency and the second set of stimulation parameters comprises a second frequency, wherein the first and second frequencies are different.

42. The method of claim 39, wherein the first set of stimulation parameters comprises a first frequency and the second set of stimulation parameters comprises a second frequency, wherein the first and second frequencies are equivalent.

43. The method of claim 39, wherein the first set of stimulation parameters comprises a first frequency and the second set of stimulation parameters comprises a second frequency, wherein the first and second frequencies activate neural fibers that engage multiple mechanisms to provide pain relief.

44. The method of claim 39, wherein the first set of stimulation parameters comprises a frequency of between 4-20 Hz targeting motor nerve fibers in a motor nerve or mixed motor and sensory nerve resulting in cyclical muscle twitches and/or muscle contractions and indirectly activate proprioceptive afferent pathways.

45. The method of claim 44, wherein the second set of stimulation parameters comprises a frequency of between 60-120 Hz to activate directly afferent pathways.

46. The method of claim 39, wherein the electrical stimulation engages in both indirect and direct afferent pathways via combinatory motor stimulation and sensory stimulation.

47. The method of claim 39, wherein the electrical stimulation engages in indirect and/or direct afferent pathways via combinatory motor stimulation and sensory stimulation.

48. The method of claim 39, wherein the first set of stimulation parameters comprise a stimulation frequency low enough to evoke visible muscle twitches and/or muscle contraction of a targeted muscle innervated by the nerve or nerve branch, with or without a modulating envelope.

49. The method of claim 48, wherein the stimulation frequency is between 0.05 and 40 Hz.

50. The method of claim 39, wherein the second set of stimulation parameters comprise a frequency high enough to paresthesia in the region of pain with or without a modulating envelope.

51. The method of claim 50, wherein the stimulation frequency is between 12 and 500 Hz.

52. The method of claim 39, wherein either the first or the second set of stimulation parameters comprise a frequency high enough to produce a neural effect that is sub-perception in the region of pain with or without a modulating frequency.

53. The method of claim 50, wherein the stimulation frequency is between 500 and 15000 Hz.

54. The method of claim 39, wherein either the first or the second set of stimulation parameters are applied intermittently at frequencies to produce patient sensation and muscle twitch without muscle fatigue.

55. The method of claim 54, wherein the frequencies are between 12 and 1500 Hz.

56. The method of claim 39, wherein the plurality of electrodes provide a coordinated multimodal signal to mimic coordination between motor and sensory signals.

57. The method of claim 39, wherein a first electrode of the plurality of electrodes delivers stimulation to activate large diameter primary afferent fibers.

58. The method of claim 57, wherein a second electrode of the plurality of electrodes delivers stimulation to activate large diameter efferent motor fibers.

59. The method of claim 58, wherein the second electrode of the plurality of electrodes delivers stimulation to generate indirect proprioceptive afferent signals.

60. The method of claim 59, wherein coordination between afferent, efferent, and proprioceptive afferent signals avoid overlapping signals to avoid nerve fatigue.

61. The method of claim 59, wherein coordination between afferent, efferent, and proprioceptive afferent signals avoid overlapping signals to avoid tissue damage.

62. The method of claim 59, wherein coordination between afferent, efferent, and proprioceptive afferent signals avoid overlapping signals to avoid painful stimulation.

63. The method of claim 59, wherein coordination between afferent, efferent, and proprioceptive afferent signals avoid overlapping signals to avoid off-target activation.
